(19) United States
(12) Patent Application Publication
(10) Pub. No.: US 2002/0129385 A1
(43) Pub. Date: Sep. 12, 2002

(54) MEDTHODS FOR IMPROVING LONG-TERM MEMORY STORAGE AND RETRIEVAL

(76) Inventors: ISABELLE M. MANSUY, PARAMUS, NJ (US); DANNY G. WINDER, NEW YORK, NY (US); MARK MAYFORD, SAN DIEGO, CA (US); MARY ELIZABETH BACH, BRONX, NY (US); ERIC R. KANDEL, RIVERDALE, NY (US)

Correspondence Address: JOHN P WHITE, COOPER & DUNHAM, 1185 AVENUE OF THE AMERICAS, NEW YORK, NY, 10036

(21) Appl. No.: 09/135,521

(22) Filed: Aug. 17, 1998

(51) Int. Cl. A61K 48/00; G01N 33/567; A01K 67/00

(52) U.S. Cl. 800/3; 514/44; 514/2; 435/7.21

(57) ABSTRACT

The present invention provides for a transgenic nonhuman mammal whose germ or somatic cells contain a nucleic acid molecule which encodes calcineurin or a variant thereof under the control of a regulatable promoter, introduced into the mammal, or an ancestor thereof, at an embryonic stage. The present invention also provides for a method of evaluating whether a compound is effective in improving long-term memory in a subject suffering from impaired long-term memory which comprises: (a) administering the compound to the transgenic nonhuman mammal of claim 1 wherein the mammal has increased brain-specific calcineurin activity due to expression of the nucleic acid, and (b) comparing the long-term memory of the mammal in step (a) with the long-term memory of the mammal in the absence of the compound so as to determine whether the compound is effective in rescuing the long-term memory defect in the subject.

The invention disclosed herein was made with Government support under Grant No. T32N507062-21 from National Institutes of Mental Health. Accordingly, the U.S. Government has certain rights in this invention.

### BACKGROUND OF THE INVENTION

Throughout this application, various publications are referenced by author and date. Full citations for these publications may be found listed alphabetically at the end of the specification immediately preceding the claims. The disclosures of these publications in their entireties are hereby incorporated by reference into this application in order to more fully describe the state of the art as known to those skilled therein as of the date of the invention described and claimed herein.

Long-lasting modifications of synaptic transmission are thought to play roles in a variety of brain functions. As a result, an intensive search has been carried out in invertebrates and vertebrates to identify the molecular components of synaptic plasticity. Much of this search has focussed on two types of synaptic enhancement: long-term facilitation in Aplysia and long-term potentiation (LTP) in hippocampus. Both of these forms of synaptic plasticity last from minutes to days, depending on the strength and number of the inducing stimuli. A major theme emerging from these studies is that protein kinases play key roles in long-term synaptic enhancement (for review, see Roberson et al., 1996). Thus, reduction of kinase activity through both pharmacological and genetic means impairs the induction or maintenance of both long-term facilitation in Aplysia and of LTP in the hippocampus (for review, see Roberson et al., 1996; Huang et al., 1996; Abel et al., 1997; Martin et al., 1997; Mayford et al., 1997). While much attention has been focused on kinases in synaptic plasticity, relatively little attention has been paid to phosphatases. Yet, phosphatases are likely to have signaling roles in synaptic plasticity that equal in importance those of kinases, if only because of their antagonistic relationship. Furthermore, most cellular models of learning postulate erasure mechanisms designed to counteract long-lasting synaptic enhancement. Consistent with this idea, recent experiments have shown that whereas brief high frequency stimulation of the Schaffer collateral pathway in the hippocampus leads to LTP, prolonged low frequency stimulation (LFS) of this same pathway results in long-term depression (LTD) of synaptic transmission, and experiments with pharmacological inhibitors suggest an important role for phosphatases in LTD (Mulkey et al. 1994; for review see Bear and Abraham, 1996). Despite the potential importance of phosphatases for synaptic plasticity, however, the study of phosphatases in hippocampus has been limited by the lack of specificity of the pharmacological inhibitors available (for example, see Helekar and Patrick, 1997), as well as by the long periods of preincubation often necessary for the inhibitors to produce alterations of synaptic function (Mulkey et al., 1994; Bear and Abraham, 1996). As a result, the roles of phosphatases in synaptic plasticity are not clear. For example, while several experiments suggest that pharmacological inhibitors of phosphatases have no effect, or enhance LTP (Blitzer et al., 1995, Mulkey et al., 1994; Muller et al., 1995; Wang and Kelly, 1996), other studies report that these inhibitors block LTP (Wang and Stelzer, 1994; Lu et al., 1996).

### SUMMARY OF THE INVENTION

The present invention provides for a transgenic nonhuman mammal whose germ or somatic cells contain a nucleic acid molecule which encodes calcineurin or a variant thereof under the control of a regulatable promoter, introduced into the mammal, or an ancestor thereof, at an embryonic stage. The present invention also provides for a method of evaluating whether a compound is effective in improving long-term memory in a subject suffering from impaired long-term memory which comprises: (a) administering the compound to the transgenic nonhuman mammal of claim 1 wherein the mammal has increased brain-specific calcineurin activity due to expression of the nucleic acid, and (b) comparing the long-term memory of the mammal in step (a) with the long-term memory of the mammal in the absence of the compound so as to determine whether the compound is effective in rescuing the long-term memory defect in the subject.

### BRIEF DESCRIPTION OF THE FIGURES

FIGS. 1A-1D. Calcineurin transgene is expressed in the hippocampus of CN98 mutant mice. FIG 1A. Schematic representation of the calcineurin transgene construct. FIG. 1B. Northern blot analysis of total RNA from CN98 mice. FIG. 1C. Enzyme activity determined in hippocampal extracts from CN98 mice. Dephosphorylation of α ³²p substrate peptide was measured in the absence or presence of the Ca²⁽ chelator EGTA. Values are mean±SEM. Wild-type (n=6); CN98 mutant (n=4), p<0.001; CN98 wild-type+EGTA (n=6); CN98 mutant+EGTA (n=4), p>0.05. FIG. 1D. In situ hybridization of calcineurin transgene in CN98 mice.

FIGS. 2A-2F. Basal synaptic transmission and short term forms of synaptic plasticity are not dramatically altered by overexpression of calcineurin. FIG. 2A. Input-output curve of fEPSP slope (mV/ms) versus stimulus (V) at the SC-CA1 pyramidal cell synapse in CN98 mutant and wild-type mice. Data are presented as mean±SEM. FIG. 2B. Plot of presynaptic fiber volley amplitude (PSFV, mV) versus fEPSP slope at the SC-CA1 pyramidal cell synapse from a random sample of slices from CN98 mutant and wild-type mice. FIG. 2C. Input-output curve of fEPSP slope (mV/ms) versus stimulus (V) at the SC-CA1 pyramidal cell synapse in CN98 mutant (13 slices, 4 mice) and wild-type (16 slices 4 mice) mice in the presence of the non-NMDA glutamate receptor antagonist DNQX (10 μM) and reduced MgSO₄ (50 μM). Data are presented as mean±SEM. Inset shows representative NMDA receptor-mediated synaptic responses during a one second, 100 Hz tetanus in wild-type and mutant slices. Scale bar is 50 ms and 5 mV. FIG. 2D. Comparison of PTP in CN98 mutant and wild-type mice. Data are presented as mean±SEM of the normalized fEPSP slope. FIG. 2E. Comparison of PPF in CN98 mutant and wild-type. Data are presented as the mean±SEM of the facilitation of the second response relative to the first response of 16 slices from 7 wild-type mice and 15 slices from 6 mutant mice. FIG. 2F. Comparison of LTD induced by 15 minutes of 1 Hz stimulation in CN98 wild-type and mutant mice aged 3-4 weeks. Data are presented as mean±SEM of the normalized fEPSP slope.

FIGS. 3A-3D. Overexpression of calcineurin inhibits L-LTP induced by four 100 Hz trains but not E-LTP induced by one 100 Hz train. Effect of overexpression of calcineurin on LTP in CN98 wild-type and mutant animals. LTP elicited by (FIG. 3A) a single 100 Hz train of one second duration, or (FIG. 3B) four 100 Hz trains spaced by five minute intervals. Each point in the time courses represents the mean fEPSP slope±SEM normalized to the average of the pretetanus fEPSP slope. Insets show representative fEPSP traces just before tetanus and FIG. 3A) 1 hour or FIG. 3B) 3 hours after. Scale bars are 2.5 mV and 10 ms. (FIGS. 3C and 3D): Drug was added at the time indicated in both panels at a concentration of 100 mM. Insets show representative fEPSP traces just before drug addition and 3 hours after. Scale bars are 2.5 mV and 10 ms. In (FIG. 3C), the decrease in the fEPSP slopes elicited towards the end of Sp-cAMPS application has been previously demonstrated to reflect a transient A1-adenosine receptor-mediated decrease in glutamate release (Bolshakov et al., 1997).

FIGS. 4A-4F. Effects of protein synthesis and PKA inhibitors on four train and two train LTP. FIG. 4A. LTP induced by four 100 Hz trains in the presence of anisomycin (30 mM) or KT5720 (1 mM) in wild-type mouse hippocampal slices. Drugs were added beginning 15 minutes prior to the first tetanus, and were washed out 15 minutes after the last tetanus. Each point in the time course represents the mean fEPSP slope±SEM normalized to the average of the pretetanus fEPSP slope. FIG. 4B. Effects of prolonged anisomycin pretreatment on LTP induced by four 100 Hz trains. Anisomycin (30 mM) was added 60 minutes prior to the first tetanus, and was washed out 15 minutes after the last tetanus. No drug: 10 slices, 8 mice; Anisomycin 4 slices, 4 mice. FIG. 4C. LTP induced by two 100 Hz trains, with a 20 second interstimulus interval, in the presence or absence of anisomycin (30 mM) in wild-type hippocampal slices. No drug: 8 slices, 5 mice; Anisomycin 7 slices, 4 mice. FIG. 4D. Effect of the PKA inhibitor KT5720 (1 mM) on LTP induced by two 100 Hz trains in wild-type hippocampal slices. FIG. 4E. LTP induced by two 100 Hz trains in hippocampal slices from CN98 mutant and wild-type mice. FIG. 4F. Effect of the PKA inhibitor KT5720 (1 mM) on LTP induced by two 100 Hz trains hippocampal slices from CN98 mutant mice.

FIGS. 5A-5E. LTP induced by two and four train (FIGS. 5B and 5C), but not one train (FIG. 5A), protocols is reduced in wild-type mice and mice overexpressing the calcineurin transgene with the tTA system. FIG. 5A. Wild-type: 14 slices, 9 mice; Tet-CN279 mutants: 6 slices, 3 mice; Tet-CN273 mutants: 4 slices, 3 mice. FIG. 5B. Wild-type: 7 slices, 4 mice; Tet-CN273 mutants: 6 slices, 3 mice. FIG. 5C. Wild-type: 10 slices, 8 mice; Tet-CN279 mutants: 7 slices, 4 mice. FIG. 5D. Calyculin A (750 nM) rescues the deficit in LTP induced by two 100 Hz trains in Tet-CN279 mutant mice. Each point in the time courses represents the mean fEPSP slope±SEM normalized to the average of the pretetanus slope. Wild-type (7 slices, 4 mice), mutant with calyculin A pretreatment (6 slices, 3 mice), wild-type with calyculin A pretreatment (6 slices, 3 mice). The data of mutant mice without drug are those illustrated in FIG. 5E. FIG. 5E. The LTP deficit seen in slices from Tet-CN279 mutants can be reversed by suppressing expression of the transgene with doxycycline.

FIGS. 6A-6D. Basal synaptic transmission and short term forms of synaptic plasticity are not altered by overexpression of calcineurin with the tTA system. FIG. 6A. Input-output curve of fEPSP slope (mV/ms) versus stimulus (V) at the SC-CA1 pyramidal cell synapse in Tet-CN279 (9 slices, 4 mice) and Tet-CN273 (20 slices, 7 mice) mutant and wild-type (21 slices, 9 mice) mice. Data are presented as mean±SEM. FIG. 6B. Input-output curve of fEPSP slope (mV/ms) versus stimulus intensity (V) at the SC-CA1 pyramidal cell synapse in Tet-CN279 (8 slices, 4 mice) and Tet-CN273 (8 slices, 4 mice) mutant and wild-type (21 slices, 8 mice) mice in the presence of the non-NMDA glutamate receptor antagonist DNQX (10 (M) and reduced MgSO₄ (50 (M) . FIG. 6C. Comparison of PTP in Tet-CN278 (6 slices, 3 mice) and Tet-CN273 (8 slices, 4 mice) mutant and wild-type (15 slices, 8 mice) mice. FIG. 6D. Comparison of PPF in Tet-CN273 (9 slices, 4 mice) and Tet-CN279 (13 slices, 4 mice) mutant and wild-type (27 slices, 10 mice). Data are presented as the mean±SEM of the facilitation of the second response relative to the first response.

FIGS. 7A-7B. A PKA-dependent, protein synthesis independent phase of LTP, I-LTP exists in mouse hippocampus. Schematic representation of the time course of potentiation induced by one train (FIG. 7B) and four-train (FIG. 7B) protocols.

FIGS. 8A-8C. CN98 mutant mice have impaired spatial memory on the Barnes maze when tested with one trial a day, but have normal memory on a cued version of the maze. FIG. 8A. Percentage of CN98 mice that acquired the spatial and cued versions of the Barnes maze with 1 trial a day. FIG. 8B. Mean number of errors made by CN98 mice on the spatial version of the Barnes maze with 1 trial a day. FIG. 8C. Mean number of errors made by CN98 mice on the cued version of the Barnes maze with 1 trial a day.

FIGS. 9A-9C. CN98 mutant mice have a normal spatial memory on the Barnes maze with four trials a day. FIG. 9A. Percentage of CN98 mice that acquired the spatial version of the Barnes maze with four trials a day. FIG. 9B. Mean number of trials and days to acquisition for CN98 mice on the spatial version of the Barnes maze with either one or four trials a day. FIG. 9C. Mean number of errors made by CN98 mice on the spatial version of the Barnes maze with four trials a day.

FIG. 10. CN98 mutant mice have normal short-term memory on the novel object recognition task. A preference index (PI) greater than 100 indicates preference for the novel object during testing. A PI equal to 100 indicates no preference whereas a PI inferior to 100 indicates a preference for the familiar object.

FIGS. 11A-11C. Regulated expression of calcineurin transgene with the tTA system. FIG. 11A. Strategy to obtain doxycycline-regulated expression of calcineurin transgene in mice. Mice from line B carry the CaMKIIα promoter-tTA transgene and mice from lines CN279 and CN273, the tetO promoter-ΔCaM-AI transgene. Both transgenes are introduced into the same mouse through mating to generate Tet-CN279 and Tet-CN273 mice. In Tet-CN279 and Tet-CN273 mice, expression of the calcineurin transgene is activated by tTA and can be repressed by doxycycline. FIG. 11B. Northern blot analysis of total forebrain RNA from Tet-CN279 and Tet-CN273 wild-type and mutant mice on or off doxycycline and RT-PCR of total forebrain RNA from Tet-CN279 and Tet-CN273 wild-type, CN279 and CN273 mice, Tet-CN279 and Tet-CN273 mutant mice on or off doxycycline. FIG. 11C. Enzyme activity determined in hippocampal extracts from Tet-CN279 and Tet-CN273 mice on or off doxycycline. Dephosphorylation of a radiolabeled peptide substrate was measured in absence or presence of the Ca²⁺ chelator EGTA in Tet-CN279 and Tet-CN273 wild-type and mutant mice on or off doxycycline. Values are mean±SEM. wild-type (Tet-CN279+Tet-CN273 ): 3.58±0.26 nmol Pi/min/mg, n=6; Tet-CN279 mutant: 7.78±0.70 nmol Pi/min/mg, n=4, p<0.0001; Tet-CN273 mutant: 8.39±0.39 nmol Pi/min/mg, n=3, p<0.001; Tet-CN279 mutant on dox: 3.95±0.48 nmol Pi/min/mg, n=4, p>0.05; Tet-CN273 mutant on dox: 4.23±0.36 nmol Pi/min/mg, n=3, p>0.05; wild-type (Tet-CN279+Tet-CN273 )+EGTA: 0.432±0.11 nmol Pi/min/mg, n=7; mutant (Tet-CN279+Tet-CN273 )+EGTA: 0.287±0.17 nmol Pi/min/mg, n=7, p>0.05.

FIG. 12. The expression of calcineurin transgene is primarily restricted to CA1 subfield in the hippocampus of Tet-CN279 and Tet-CN273 mutant mice and is repressed by doxycycline. Regional distribution of calcineurin transgene determined by in situ hybridization on mouse brain sagittal sections from Tet-CN279 wild-type, Tet-CN279 and Tet-CN273 mutant on or off doxycycline.

FIGS. 13A-13G. CN98 and Tet-CN279 mutant mice do not use the spatial search strategy. FIG. 13A. Representative examples of the search strategies employed on the spatial version of the Barnes circular maze task. FIGS. 13B-13G. Use of random search strategy by CN98 (FIG. 13B) and Tet-CN279 (FIG. 13C) mice, of serial search strategy by CN98 (FIG. 13D) and Tet-CN279 (FIG. 13E) mice and of spatial search strategy by CN98 (FIG. 13F) and Tet-CN279 (FIG. 13G) mice.

FIGS. 14A-14E. Induced gene expression in mouse brain with the rtTA system. FIG. 14A. Strategy to obtain doxycycline-induced expression of lacZ reporter or calcineurin transgene in mouse brain. Mice from line 1237 carry the CaMKIIα promoter-rtTA transgene; from line lac1, the tetO promoter-lacZ transgene and from line CN279, the tetO promoter-ΔCaM-AI transgene. Double transgenic (mutant) mice (rTet-lacZ and rTet-CN279 ) were obtained by crossing mice from line 1237 with mice from either line lac1 rTet-lacZ or CN279 rTet-CN279. In mutant mice, the expression of the lacZ reporter or the calcineurin transgene is induced by rtTA in the presence of doxycycline. FIG. 14B. Forebrain-specific induction of lacZ reporter gene with the rtTA system. Sagittal section from adult rTet-lacZ mutant mouse not treated (Off) or treated (On) with doxycycline for 6 days at 6 mg/g food and stained with X-gal. FIG. 14C. Pattern of calcineurin transgene expression in rTet-CN279 mutant mice. In situ hybridization performed on sagittal brain sections from adult rTet-CN279 mutant mouse not treated (Off) or treated (On) with doxycycline (6 days of 6 mg doxycycline/g food). FIG. 14D. LacZ gene expression after 3 days of treatment with 6 mg/g of doxycycline (on, 3 days). FIG. 14E. Calcineurin transgene expression after 3 days of treatment with 6 mg/g of doxycycline (on, 3 days).

FIGS. 15A-15B. Regulation of the calcineurin transgene expression with the rtTA system. Determination of the calcineurin transgene expression by Northern blot analysis in forebrain (FIG. 15A) and phosphatase activity assay in hippocampus (FIG. 15B) from adult rTet-CN279 control mice not treated or treated for 2 weeks with doxycycline at 6 mg/g food (Control, 4.85±0.76 nmol Pi/min/mg, n=4, pooled data), mutant mice not treated with doxycycline (Mutant, 4.89±1.02 nmol Pi/min/mg, n=3) or treated with doxycycline for 2 weeks at 6 mg/g food (Mutant dox, 8.63±1.17 nmol Pi/min/mg, n=3, p<0.05 compared to control by t-test) and in mutant mice withdrawn from doxycycline for 2 weeks after a 2-week treatment with 6 mg/g doxycycline in the food (Mutant on-off dox, 5.15±0.83_nmol Pi/min/mg, n=3). Phosphatase activity was blocked by the Ca²⁺ chelator EGTA in extracts from control and mutant mice not treated or treated with doxycycline suggesting that the measured phosphatase activity is Ca²⁺-dependent. Values are means±SEM.

FIGS. 16A-16D. The induction of the calcineurin transgene expression leads to a reversible defect in I-LTP in hippocampal CA1 Schaffer collateral pathway. FIG. 16A. Input-output curve of fEPSP slope (mV/ms) versus stimulus strength (V) at the Schaffer collateral-CA1 pyramidal cell synapse in hippocampal slices from untreated rTet-CN279 control and mutant mice perfused with ACSF alone (Control, 9 slices, 5 mice; Mutant, 18 slices, 5 mice) or control and mutant mice treated with doxycycline and perfused with ACSF containing 6 ng/ml doxycycline (Control dox, 15 slices, 5 mice; Mutant dox, 14 slices, 4 mice). Data are means±SEM. FIG. 16B. One 100 Hz 1 sec train was used to induce E-LTP in hippocampal slices from rTet-CN279 control and mutant mice treated with doxycycline and perfused with ACSF containing 6 ng doxycycline/ml (Control dox, 8 slices, 3 mice; Mutant dox, 9 slices, 3 mice). FIG. 16C. Two 100_Hz 1 sec trains separated by 20 sec were used to induce I-LTP in hippocampal slices from rTet-CN279 control and mutant mice not treated with doxycycline and perfused with ACSF alone (Control, 8 slices, 4 mice; Mutant, 9 slices, 3 mice) or treated with doxycycline and perfused with ACSF containing 6 ng doxycycline/ml (Control dox, 18 slices, 8 mice; Mutant dox, 13 slices, 6 mice). FIG. 16D. The I-LTP defect is rescued by doxycycline withdrawal in rTet-CN279 mutant mice. Two 100 Hz 1 sec trains induced normal I-LTP in control and mutant mice withdrawn from doxycycline for 2 weeks after a 2-week treatment (Control on-off dox, 6 slices, 3 mice; Mutant on-off dox, 6 slices, 3 mice).

FIG. 17. Diagram illustrating behavioral training, testing and doxycycline treatment in the Morris water maze.

For the visible platform version of the Morris water maze, mice were trained for 2 days with 4 trials per day then were either tested for retention 2 weeks later or trained on the hidden platform version of the task. For retention on the visible platform task, mice were kept for 2 weeks after training was completed, treated or not treated with doxycycline during this period, then retested with 4 trials on testing day. For the hidden platform version of the task, mice were trained for 5 days with 4 trials a day, tested on a first probe trial then after 2-week retention during which they were either treated or not treated with doxycycline, were tested on a second probe trial. A third probe trial was performed 2-3 weeks after the second one and mice treated only between the first and second probe trials were withdrawn from doxycycline during these 2-3 weeks. For both the visible and hidden platform versions of the task, mice were either not administered doxycycline (Control or mutant), administered doxycycline only during the 2 week retention immediately after training (Control or mutant off-on-off dox) or across training, retention and testing (Control or mutant on dox).

FIGS. 18A-18C. Spatial but not non-spatial learning is impaired in mutant rTet-CN279 mice expressing the calcineurin transgene in the Morris water maze. FIG. 18A. Performance of rTet-CN279 mice on the visible platform version of the task during training. Escape latencies were plotted across the 2-day training (day 1 and day 2) for control mice not treated (Control, n=21) or treated (Control dox, n=20) with doxycycline one week before and accross training and for mutant mice not treated (Mutant, n=16) or treated (Mutant dox, n=8) with doxycycline one week before and accross training. Values are group means±SEM. FIG. 18B. Retrieval on the visible platform version of the task. Mice were tested with 4 trials on day 3 after a 2-week retention period. Mice were administered doxycycline either only during the 2 week retention and testing (Control off-on dox, n=4; Mutant off-on dox, n=5) or across training, retention and testing (Control dox, n=5; Mutant dox, n=3). Values are means±SEM. A three way ANOVA with group, day and trial as factors revealed no significant effect involving group across training (A) and testing (B) but a significant effect involving trial. FIG. 18C. Performance of rTet-CN279 mice on the hidden platform version of the Morris water maze during training. Escape latencies were plotted across the 5-day training for control mice not treated (Control, n=17) or treated (Control dox, n=15) with doxycycline one week before and accross training and for mutant mice not treated (Mutant, n=11) or treated (Mutant dox, n=5) with doxycycline one week before and accross training. Values are group means±SEM. ANOVAs revealed a main effect of group overall (F[3,44]=5.99, p<0.01) and on day 4 F[3,44]=2.99, p<0.05), when the mutant dox group was significantly-different from each of the other groups by a least significant difference multiple range test (p<0.05 in each case).

FIGS. 19A-19D. The storage and retrieval of spatial memory is impaired by the calcineurin transgene expression in the Morris water maze. FIG. 19A. Performance of rTet-CN279 mice during the first probe trial. The percent of time spent in each quadrant of the pool was determined for rTet-CN279 control and mutant mice not treated or treated with doxycycline one week before training and accross training (Control, n=17; Control dox, n=15; Mutant, n=11; Mutant dox, n=5). Values are means±SEM. A two-way ANOVA revealed a significant interaction of quadrant by group (F[9, 132]=3.43, p<0.01) and subsequent one-way ANOVAS and range tests revealed that performance on the training quadrant (TQ) was significantly different from performance on the other quadrants for both control groups and for the mutant group (p<0.05 in each case) but not for the mutant dox group. On TQ, a one-way ANOVA revealed a main effect of group (F[3,44]=4.52, p<0.01) and a subsequent range test revealed that the mutant dox group was significantly different from each of the other groups (p<0.05 in each case). FIG. 19B. Number of platform crossings in each quadrant during the first probe trial for control and mutant mice not treated or treated with doxycycline one week before training and across training (Control, n=17; Control dox, n=15; Mutant, n=11; Mutant dox, n=5). Values are means±SEM. A two-way ANOVA revealed a significant interaction of quadrant by group (F[9, 129]=4.10, p<0.01) and subsequent one-way ANOVAS and range tests revealed that performance on TQ was significantly different from performance on the other quadrants for both control groups and for the mutant group (p<0.05 in each case) but not for the mutant dox group. On TQ, a one-way ANOVA revealed a main effect of group (F[3,43]=4.08, p<0.05) and a subsequent range test revealed that the mutant dox group was significantly different from each of the other groups (p<0.05 in each case). FIG. 19C. Performance of rTet-CN279 mice during the second probe trial. The percent of time spent in each quadrant of the pool was determined for control mice treated or not treated with doxycycline (pooled) (Control, n=31), mutant mice treated only during the 2-week retention after the first probe trial (Mutant off-on dox, n=9), and for mutant mice treated with doxycycline one week before training, during training and during the 2-week retention (Mutant dox, n=5). Values are means±SEM. A two-way ANOVA revealed a significant interaction of quadrant by group (F[6, 129]=2.67, p<0.05) and subsequent one-way ANOVAS and range tests revealed that performance on TQ was significantly different from performance on the other quadrants for the control group (p<0.05 in each case) but not for the mutant off-on dox and mutant dox groups. On TQ, a one-way ANOVA revealed a main effect of group (F[2,42]=4.41, p<0.05) and a subsequent range test revealed that the control group was significantly different from each of the other groups. FIG. 19D. Summary of performance on QT across probe trials. rTet-CN279 mice in the training quadrant during the first, second and third probe trials. The time spent in the training quadrant was plotted across probe trials. A two-way ANOVA revealed a significant effect of group (F[2,45]=17.65, p<0.01) and a significant group by trial interaction (F[4,75]=3.69, p<0.01). A one-way ANOVA and subsequent range test for the mutant off-on-off dox group revealed that performance on the second probe trial was significantly different from performance on each of the other trials for that group. All values are mean±SEM.

### DETAILED DESCRIPTION OF THE INVENTION

The present invention provides for a transgenic nonhuman mammal whose germ or somatic cells contain a nucleic acid molecule which encodes calcineurin or a variant thereof under the control of a regulatable promoter, introduced into the mammal, or an ancestor thereof, at an embryonic stage.

In one embodiment, the regulatable promoter is responsive to a transactivator. In one example, the regulatable promoter is a tetO promoter. In another example, the transactivator is doxycycline. In another example, the transactivator is encoded by a gene under the control of a forebrain specific promoter. In one embodiment, the forebrain-specific promoter is a murine CaMKIIα promoter.

In a further embodiment, the transgenic nonhuman mammal may be a mouse, a rat, a sheep, a bovine, a canine, a porcine or a primate.

The present invention also provides for a screening assay for evaluating whether a compound is effective in improving long-term memory in a subject suffering from impaired long-term memory which comprises: (a) administering the compound to a transgenic nonhuman mammal wherein the mammal has increased brain-specific calcineurin activity, and (b) comparing the long-term memory of the mammal in step (a) with the long-term memory of the mammal in the absence of the compound so as to determine whether the compound is effective in rescuing the long-term memory defect thereby improving the long-term memory of the subject.

In embodiments of this screening assay, the subject may a human, a rat, a mouse, a sheep, a bovine, a canine, a porcine or a primate. In another embodiment, the compound identified by the screening assay is an organic compound, a peptide, an inorganic compound, a lipid or a small synthetic compound. In a further embodiment, the transgenic nonhuman mammal utilized in the screening assay is a genetically modified mouse with increased calcineurin activity in brain. For example, the transgenic nonhuman mammal is a lac1 mouse, a 1237 mouse, a CN98 mouse, a CN279 mouse, an rTet-lacZ mouse, or an rTet-CN279 mouse.

In a further embodiment, the impaired long-term memory of the subject is due to amnesia, Alzheimer's disease, amyotrophic lateral sclerosis, a brain injury, cerebral senility, chronic peripheral neuropathy, a cognitive disability, a degenerative disorder associated with a learning and memory deficit, defective synaptic transmission, Down's Syndrome, dyslexia, electric shock induced amnesia, Guillain-Barre syndrome, head trauma, stroke, cerebral ischemia, Huntington's disease, a learning disability, a memory deficiency, memory loss, a mental illness, mental retardation, memory or cognitive dysfunction, multi-infarct dementia, senile dementia, myasthenia gravis, a neuromuscular disorder, Parkinson's disease, Pick's disease, a reduction in spatial memory retention, senility, Tourrett's syndrome, caridac arrest, open heart surgery, chronic fatigue syndrome, major depression or electroconvulsive therapy.

The present invention also provides for a method for improving long-term memory storage and retrieval in a subject suffering from a long-term memory defect which comprises administering to the subject a compound capable of reversing a defect in intermediate-long-term-potentiation (I-LTP) in the subject thereby improving long-term memory storage and retrieval.

The present invention further provides for a method for improving long-term memory in a subject suffering from a long-term memory defect which comprises administering to the subject a compound identified by the screening assay as effective in improving long-term memory.

The present invention also provides for a method for improving long-term memory in a subject suffering from a long-term memory defect which comprises administering to the subject a compound that inhibits calcineurin activity in the forebrain of the subject thereby improving long-term memory in the subject. In another embodiment, the present invention provides for a method for improving long-term memory in a subject suffering from a long-term memory defect which comprises administering to the subject an amount of a compound that modifies a calcineurin-dependent biochemical pathway in the forebrain of the subject, effective to modify such pathway and thereby improve long-term memory in the subject.

The present invention encompasses treating a subject suffereing from impaired long-term memory. For example, the impaired long-term memory of the subject is due to amnesia, Alzheimer's disease, amyotrophic lateral sclerosis, a brain injury, cerebral senility, chronic peripheral neuropathy, a cognitive disability, a degenerative disorder associated with a learning and memory deficit, defective synaptic transmission, Down's Syndrome, dyslexia, electric shock induced amnesia, Guillain-Barre syndrome, head trauma, stroke, cerebral ischemia, Huntington's disease, a learning disability, a memory deficiency, memory loss, a mental illness, mental retardation, memory or cognitive dysfunction, multi-infarct dementia, senile dementia, myasthenia gravis, a neuromuscular disorder, Parkinson's disease, Pick's disease, a reduction in spatial memory retention, senility, Tourrett's syndrome, caridac arrest, open heart surgery, chronic fatigue syndrome, major depression or electroconvulsive therapy.

In one embodiment, the compound administered to the subject may be an organic compound, a peptide, an inorganic compound, a lipid or a small synthetic compound.

In another embodiment, the subject is a human, a rat, a mouse, a sheep, a bovine, a canine, a porcine or a primate.

In a further embodiment of the present invention, the administration is via an aerosol, oral delivery, intravenous delivery, an inhalent, an eyedrop, topical delivery, a time-release implant or an intraspinal injection. The implant may be subcutaneous.

The present invention also provides for a compound identified by the screening assay as effective in improving long-term memory. The compound may be a known compound for which a new use is identified or the compound may be a previously unknown compound.

The present invention also provides for a pharmaceutical composition comprising the compound and a carrier. For example, the carrier is an aerosol, topical, intravenous or oral carrier, or a subcutaneous implant. In another embodiment, the implant may be a time release implant.

The present invention provides for a nucleic acid molecule which comprises: (i) a CaMKIIα promoter sequence or fragment thereof, and (ii) a nucleic acid sequence encoding a tetracycline-controlled transcriptional activator protein flanked by an artificial intron sequence and splice site sequence in the 5′ direction and by a polyadenylation signal sequence in the 3′ direction.

In one embodiment, the nucleic acid sequence of (i) is the sequence of the 8.5 kb CaMKII promoter insert of plasmid pMM403+CAM (from ATCC Accession No.______).

In another embodiment, the nucleic acid sequence of (ii) is the sequence of the 1.04 kb insert of plasmid pMM403+rtTA (from ATCC Accession No.______).

In a further embodiment, the nucleic acid sequence of (ii) is a rtTA sequence. In a further embodiment, (i) is upstream from (ii).

The present invention provides for a replicable vector which comprises the nucleic acid molecule described herein and for a host cell which comprises the replicable vector.

The present invention also provides for a nucleic acid molecule which comprises: (i) a transcriptional activator protein-responsive promoter sequence; (ii) a nucleic acid sequence encoding the Au catalytic subunit of calcinuerin or a variant thereof; (iii) a polyadenylation signal sequence.

In one embodiment, the nucleic acid sequence of (i) is the sequence of the 1.04 kb insert of plasmid pMM403+rtTA (from ATCC Accession No.______). In another embodiment, the nucleic acid sequence of (i) is the sequence of the 1197 bp insert of plasmid pMM403+CAM (from ATCC Accession No. ______). In a further embodiment, the sequence of (i) is a tetO promoter sequence. In a further embodiment, the sequence of (ii) is truncated a calcineurin ΔcaM-AI. In another embodiment, (i) is upstream of (ii) and (ii) is upstream of (iii). In another embodiment, the nucleic acid sequence of (ii) is operably linked to the promoter of (i).

The present invention also provides for a transgenic nonhuman mammal whose germ or somatic cells contain one of the nucleic acid molecule described hereinabove introduced into the mammal, or an ancestor thereof, at an embryonic stage.

The present invention also provides for a transgenic nonhuman mammal whose germ or somatic cells contain at least two of the nucleic acid molecules described hereinabove introduced into the mammal, or an ancestor thereof, at an embryonic stage.

### DEPOSITS UNDER BUDAPEST TREATY

The following transgene DNA constructs were deposited to meet the requirements of the provisions of the Budapest Treaty on the International Recognition of the Deposit of Microorganisms for the Purposes of Patent Procedure with the American Type Culture Collection, 10801 University Blvd., Manassas, Va., 20110-2209, U.S.A. on Aug. 17, 1998. The plasmids of the present invention were accorded with ATCC Accession Nos. ____, ____, and ____.

(1) pMM400+CAM—(ATCC Accession No.____) (Mayford et al. 1996, Science 274:1678). pMM400+CAM contains tetracycline promoter/Eco RI ligated with blunt EcoRI CAM fragment (calcineurin CAM 1197 bp). The total size is 6.9 kb. The vector is 5.63 kb and the insert is 1.27 kb. The plasmid is ampicillin resistant. The plasmid can be grown in any competent cells (Sure® cells from Stratagene®). This plasmid is linearized with NotI before isolating the insert and then using the isolated insert for injection into a cell in order to generate a transgenic nonhuman mammal. The NotI enzymatic digestion will produce a 3.15 kb band which is the band to be collected for injection and a 3.75 kb band. A double digestion with NotI and Sfi will produce a 3.15 kb band, a 0.16 kb band and a 3.1 kb band. The sequence of the insert which is released by NotI (1.27 kb) which is used as a reagent in creating a transgenic mammal is . . . (Seq I.D. No. 1). This sequence can be easily deduced by one of ordinary skill in the art by routine methods e.g., DNA sequencing.

(2) pMM403+CAM—(ATCC Accession No.____) (O'Keefe et al. 1992 Nature 357:692). The pMM403 is digested with NotI and the CAM (calcineurin) insert is flanked by SV40 intron and a SV40 poly A signal sequence. The total size of the plasmid is 14.6 kb. The vector is 12 kb and the insert is 1440 bp+1197 bp=2.6 kb. The CAM-Ai (calcineurin) insert is 1197 bp. There is also a CaMKII promoter in this plasmid which is 8.5 kb and can be released by a double digest of Sfi and NotI. The plasmid is resistant to ampicillin and can be propagated in any competent cells (i.e. Sure® cells, Stratgene®). The sequence of the insert which is released by NotI (2.6 kb) which is used as a reagent in creating a transgenic mammal is . . . (Seq I.D. No. 2). This sequence can be easily deduced by one of ordinary skill in the art by routine methods e.g., DNA sequencing.

(3) pMM403+rtTA—(ATCC Accession No.____) (Mayford et al. 1996 PNAS 93:13250). PMM403 was digested with NotI and rtTA gene is digested by EcoRI/BamHI from pUHG 17-1 (Zossan et al. 1995 Science 268:1766). The total size is about 13 kb. The vector is 12 kb and the insert is 1.04 kb. This vector is resistant to ampicillin and can be transformed into any competent cells (Sure® cells from Stratagene®). The rtTA insert is 1.04 kb and can be released from the vector with a NotI digestion. There is a CaMKII promoter also present in this plasmid which is 8.5 kb and can be released with a double digestion of Not I and Sfi. The sequence of the insert which is released by NotI (1.04 kb) which is used as a reagent in creating a transgenic mammal is . . . (Seq I.D. No. 3). This sequence can be easily deduced by one of ordinary skill in the art by routine methods e.g., DNA sequencing.

The maps for each plasmid listed above was provided to the ATCC with the deposit.

These three constructs are merely three examples of the DNA transgenes used to create ultimately a transgenic mouse or nonhuman mammal useful in the screening assays described herein. There are many other embodiments of such a transgene construct. The origin of the promoter, the calcineurin gene and the rtTA system may be from other species. One of ordinary skill in the art could isolate the inserts from each of these three plasmids and perform routine sequencing reactions in order to ascertain the sequence for each transgene construct. In addition, any linker DNA sequences used in the construction of each of these plasmids would be easily deduced by one of ordinary skill in the art by routine methods, e.g. DNA sequencing.

The present invention provides for compounds and pharmaceutical compositions identified by the screening method herein.

A “variant thereof” is defined herein to encompass a closely related sequence (e.g. 90%, 95%, 80%, 75%, etc. homologous) which has the same functionality as the original sequence. A variant thereof may include a fragment of the original sequence.

This invention provides a gene transfer vector, for example a plasmid or a viral vector, comprising a nucleic acid molecule encoding the light chain protein of the monoclonal antibody operably linked to a promoter of RNA transcription. This invention also provides a gene transfer vector, for example a plasmid or a viral vector, comprising a nucleic acid molecule encoding the heavy chain protein of the monoclonal antibody operably linked to a promoter of RNA transcription.

This invention provides a host vector system comprising the gene transfer vectors described and claimed herein in a suitable host cell. In one embodiment of this invention, the suitable host cell is a stably transformed eukaryotic cell, for example a stably transformed yeast or a mammalian cell. In the preferred embodiment of this invention, the stably transformed eukaryotic cell is a stably transformed mammalian cell.

In one embodiment of this invention, the nucleic acid molecule is a DNA molecule. Preferably, the DNA molecule is a cDNA molecule. The nucleic acid molecules are also valuable in a new and useful method of gene therapy, i.e., by stably transforming cells isolated from an animal with the nucleic acid molecules and then readministering the stably transformed cells to the animal. Methods of isolating cells include any of the standard methods of withdrawing cells from an animal. Suitable isolated cells include, but are not limited to, bone marrow cells. Methods of readministering cells include any of the standard methods of readministering cells to an animal.

The compound may be an organic compound, a nucleic acid, an inorganic compound, a lipid, or a small synthetic compound. The mammal may be a mouse, a rat, a sheep, a bovine, a canine, a porcine, or a primate. The subject may be a human. For the purposes of this invention, “administration” means any of the standard methods of administering a pharmaceutical composition known to those skilled in the art. The administration may comprise intralesional, intraperitoneal, intramuscular or intravenous injection; infusion; liposome-mediated delivery; gene bombardment; topical, nasal, oral, anal, ocular or otic delivery. Delivery may be via a time release object placed subcutaneously, intracranially or elsewhere within the body of the subject. The material used to fabricate the time release substance will be known to one of skill in the art and will include new materials possibly developed in the future. The purpose for effective and timely time release of a particular compound, however is known now and will be simply more effective and efficiently done with new substances.

As used herein, the term “neuronal degradation” includes morphological and functional deterioration of neuronal cells characteristic of degeneration associated with age or characteristic of an association with a neurological disorder. “Neuronal degradation” also includes cognitive impairments which may be associated with aging, Alzheimer's disease, amyotrophic lateral sclerosis, chronic peripheral neuropathy, drug or alcohol use, electroshock treatment or trauma, Guillain-Barre syndrome, Huntington's disease, a learning disability, a memory deficiency, a mental illness, myasthenia gravis, Parkinson's disease and reduction in spatial memory retention.

As used herein, the term “cognitive disorder” includes a learning disability or a neurological disorder which may be Alzheimer's Disease, a degenerative disorder associated with learning, a learning disability, memory or cognitive dysfunction, cerebral senility, multi-infarct dementia and senile dementia, electric shock induced amnesia or amnesia.

The subject may be a mammal or a human subject. The administration may be intralesional, intraperitoneal, intramuscular or intravenous injection; infusion; liposome-mediated delivery; gene bombardment; topical, nasal, oral, anal, ocular or otic delivery.

In the practice of any of the methods of the invention or preparation of any of the pharmaceutical compositions an “therapeutically effective amount” is an amount which is capable of alleviating the symptoms of the cognitive disorder of memory or learning in the subject. Accordingly, the effective amount will vary with the subject being treated, as well as the condition to be treated. For the purposes of this invention, the methods of administration are to include, but are not limited to, administration cutaneously, subcutaneously, intravenously, parenterally, orally, topically, or by aerosol.

As used herein, the term “suitable pharmaceutically acceptable carrier” encompasses any of the standard pharmaceutically accepted carriers, such as phosphate buffered saline solution, water, emulsions such as an oil/water emulsion or a triglyceride emulsion, various types of wetting agents, tablets, coated tablets and capsules. An example of an acceptable triglyceride emulsion useful in intravenous and intraperitoneal administration of the compounds is the triglyceride emulsion commercially known as Intralipid®.

Typically such carriers contain excipients such as starch, milk, sugar, certain types of clay, gelatin, stearic acid, talc, vegetable fats or oils, gums, glycols, or other known excipients. Such carriers may also include flavor and color additives or other ingredients.

This invention also provides for pharmaceutical compositions including therapeutically effective amounts of protein compositions and compounds capable of alleviating the symptoms of the cognitive disorder of memory or learning in the subject of the invention together with suitable diluents, preservatives, solubilizers, emulsifiers, adjuvants and/or carriers useful in treatment of neuronal degradation due to aging, a learning disability, or a neurological disorder. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the compound, complexation with metal ions, or incorporation of the compound into or onto particulate preparations of polymeric compounds such as polylactic acid, polglycolic acid, hydrogels, etc, or onto liposomes, micro emulsions, micelles, unilamellar or multi lamellar vesicles, erythrocyte ghosts, or spheroplasts. Such compositions will influence the physical state, solubility, stability, rate of in vivo release, and rate of in vivo clearance of the compound or composition. The choice of compositions will depend on the physical and chemical properties of the compound capable of alleviating the symptoms of the cognitive disorder of memory or the learning disability in the subject.

Controlled or sustained release compositions include formulation in lipophilic depots (e.g., fatty acids, waxes, oils). Also comprehended by the invention are particulate compositions coated with polymers (e.g., poloxamers or poloxamines) and the compound coupled to antibodies directed against tissue-specific receptors, ligands or antigens or coupled to ligands of tissue-specific receptors. Other embodiments of the compositions of the invention incorporate particulate forms protective coatings, protease inhibitors or permeation enhancers for various routes of administration, including parenteral, pulmonary, nasal and oral.

Portions of the compound of the invention may be “labeled” by association with a detectable marker substance (e.g., radiolabeled with 125I or biotinylated) to provide reagents useful in detection and quantification of compound or its receptor bearing cells or its derivatives in solid tissue and fluid samples such as blood, cerebral spinal fluid or urine.

When administered, compounds are often cleared rapidly from the circulation and may therefore elicit relatively short-lived pharmacological activity. Consequently, frequent injections of relatively large doses of bioactive compounds may by required to sustain therapeutic efficacy. Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound adducts less frequently or in lower doses than with the unmodified compound.

Attachment of polyethylene glycol (PEG) to compounds is particularly useful because PEG has very low toxicity in mammals (Carpenter et al., 1971). For example, a PEG adduct of adenosine deaminase was approved in the United States for use in humans for the treatment of severe combined immunodeficiency syndrome. A second advantage afforded by the conjugation of PEG is that of effectively reducing the immunogenicity and antigenicity of heterologous compounds. For example, a PEG adduct of a human protein might be useful for the treatment of disease in other mammalian species without the risk of triggering a severe immune response. The compound of the present invention capable of alleviating symptoms of a cognitive disorder of memory or learning may be delivered in a microencapsulation device so as to reduce or prevent an host immune response against the compound or against cells which may produce the compound. The compound of the present invention may also be delivered microencapsulated in a membrane, such as a liposome.

Polymers such as PEG may be conveniently attached to one or more reactive amino acid residues in a protein such as the alpha-amino group of the amino terminal amino acid, the epsilon amino groups of lysine side chains, the sulfhydryl groups of cysteine side chains, the carboxyl groups of aspartyl and glutamyl side chains, the alpha-carboxyl group of the carboxy-terminal amino acid, tyrosine side chains, or to activated derivatives of glycosyl chains attached to certain asparagine, serine or threonine residues.

Numerous activated forms of PEG suitable for direct reaction with proteins have been described. Useful PEG reagents for reaction with protein amino groups include active esters of carboxylic acid or carbonate derivatives, particularly those in which the leaving groups are N-hydroxysuccinimide, p-nitrophenol, imidazole or 1-hydroxy-2-nitrobenzene-4-sulfonate. PEG derivatives containing maleimido or haloacetyl groups are useful reagents for the modification of protein free sulfhydryl groups. Likewise, PEG reagents containing amino hydrazine or hydrazide groups are useful for reaction with aldehydes generated by periodate oxidation of carbohydrate groups in proteins.

In one embodiment the compound of the present invention is associated with a pharmaceutical carrier which includes a pharmaceutical composition. The pharmaceutical carrier may be a liquid and the pharmaceutical composition would be in the form of a solution. In another embodiment, the pharmaceutically acceptable carrier is a solid and the composition is in the form of a powder or tablet. In a further embodiment, the pharmaceutical carrier is a gel and the composition is in the form of a suppository or cream. In a further embodiment the active ingredient may be formulated as a part of a pharmaceutically acceptable transdermal patch.

Transgenic Mice

The nucleic acid molecules are also valuable in a new and useful method of gene therapy, i.e., by stably transforming cells isolated from an animal with the nucleic acid molecules and then readministering the stably transformed cells to the animal. Methods of isolating cells include any of the standard methods of withdrawing cells from an animal. Suitable isolated cells include, but are not limited to, bone marrow cells. Methods of readministering cells include any of the standard methods of readministering cells to an animal.

The methods used for generating transgenic mice are well known to one of skill in the art. For example, one may use the manual entitled “Manipulating the Mouse Embryo” by Brigid Hogan et al. (Ed. Cold Spring Harbor Laboratory) 1986. The transgenic nonhuman mammal may be transfected with a suitable vector which contains an appropriate piece of genomic clone designed for homologous recombination. Alternatively, the transgenic nonhuman mammal may be transfected with a suitable vector which encodes an appropriate ribozyme or antisense molecule. See for example, Leder and Stewart, U.S. Pat. No. 4,736,866 for methods for the production of a transgenic mouse.

This invention provides for improving the long-term memory of a subject.

A “reporter molecule”, as defined herein, is a molecule or atom which, by its chemical nature, provides an identifiable signal allowing detection of the circular oligonucleotide. A reporter molecule may be encoded by a reporter gene. Detection can be either qualitative or quantitative. The present invention contemplates using any commonly used reporter molecules including radionucleotides, enzymes, biotins, psoralens, fluorophores, chelated heavy metals, and luciferin. The most commonly used reporter molecules are either enzymes, fluorophores, or radionucleotides linked to the nucleotides which are used in circular oligonucleotide synthesis. Commonly used enzymes include horseradish peroxidase, alkaline phosphatase, glucose oxidase and α-galactosidase, among others. The substrates to be used with the specific enzymes are generally chosen because a detectably colored product is formed by the enzyme acting upon the substrate. For example, p-nitrophenyl phosphate is suitable for use with alkaline phosphatase conjugates; for horseradish peroxidase, 1,2-phenylenediamine, 5-aminosalicylic acid or toluidine are commonly used. The methods of using such hybridization probes are well known and some examples of such methodology are provided by Sambrook et al, 1989.

Gene Therapy

Numerous methods have been developed over the last decade for the transduction of genes into mammalian cells for potential use in gene therapy. In addition to direct use of plasmid DNA to transfer genes, retroviruses, adenoviruses, parvoviruses, and herpesviruses have been used (Anderson et al., 1995; Mulligan, 1993; The contents of whch are incorporated in their entirety into the subject application) . For transfer of genes into cells ex viva and subsequent reintroduction into a host, retroviruses have been the vectors of choice. Advantages are that infection of retroviruses is highly efficient and that the provirus generated after infection integrates stably into the host DNA. A disadvantage however, is that stable integration requires cell division, and many of the earliest hematopoietic progenitor cells that would be the preferred targets of gene therapy, do not divide under conditions used for the infections and hence to not incorporate virus, or if they do they may not retain their potential to completely reconsitute a host. Notwithstanding this problem, it is possible that the long-term culture-initiating cells that can be transduced by retroviruses may be sufficient to repopulate some compartment with cells that are particularly long lived and stable.

Most current gene therapy protocols use murine retroviral vectors to deliver therapeutic genes into target cells; this process, which is called transduction, mimics the early events of retroviral infection. The crucial difference is that, unlike replication competent retroviruses, the vector genome packaged within the viral coat contains no genes for viral proteins and therefore is incapable of replication. For example, a vector would be designed to have 3′ and 5′ long terminal repeat sequences necessary only for the integration of the viral DNA intermediate into the target host cell chromosome and a packaging signal that allows packaging into viral structural proteins supplied by the packaging line in trans (Miller, 1992; Wilson et al., 1990; The contents of which are incorporated in their entirety into the subject application). Retroviral constructs are made in which the DNA of the gene of interest (that is, the gene which one wishes to have expressed under the control of the CaMKIIα 5′ promoter, specifically localized expression to the forebrain, hippocampal regions) and is inserted downstream of the CaMKIIα promoter to generate a vector. Genomic integration is the terminal step for these defective retroviral vectors. They cannot make viral proteins in cells transduced with the packaged vector and therefore cannot produce progeny virus. The CaMKIIα promoter retroviral constructs are transfected into virus packaging cell lines to generate infectious, but non-replicating virus particles. Such virus packaging cell lines are known to those of skill in the art. Cloning procedures and retroviral infection of cell lines are well known to one skilled in the art and detailed protocols may be found in Kriegler, 1990. Producer lines with high virus titers are chosen for their ability to transduce the human neuronal cell lines resulting in expression of the gene of interest in that cell line.

There are several protocols for human gene therapy which have been approved for use by the Recombinant DNA Advisory Committee (RAC) which conform to a general protocol of target cell infection and administration of transfected cells (see for example, Blaese, R. M., et al., 1990; Anderson, W. F., 1992; Culver, K. W. et al., 1991). In addition, U.S. Pat. No. 5,399,346 (Anderson, W. F. et al., Mar. 21, 1995, U.S. Ser. No. 220,175) describes procedures for retroviral gene transfer. The contents of these support references are incorporated in their entirety into the subject application. It may be necessary to select for a particular subpopulation of originally harvested cells for use in the infection protocol. Then, a retroviral vector containing the gene(s) of interest would be mixed into the culture medium. The vector binds to the surface of the subject's cells, enters the cells and inserts the gene of interest randomly into a chromosome. The gene of interest is now stably integrated and will remain in place and be passed to all of the daughter cells as the cells grow in number. The cells may be expanded in culture for a total of 9-10 days before reinfusion (Culver et al., 1991). As the length of time the target cells are left in culture increases, the possibility of contamination also increases, therefore a shorter protocol would be more beneficial. In addition, the currently reported transduction efficiency of 10-15% is well below the ideal transduction efficiency of 90-100% which would allow the elimination of the selection and expansion parts of the currently used protocols and reduce the opportunity for target cell contamination.

In one embodiment of the method above the nucleic acid molecule is incorporated into a liposome to allow for administration to the subject. Methods of incorporation of nucleic acid molecules into liposomes are well known to those of ordinary skill in the art. In another embodiment of this method, the molecule may be delivered via transfection, injection, or viral infection. Other methods of delivery of nucleic acids and nucleic acid compositions as discussed herein include viral gene-mediated transfer, small particle bombardment, receptor-mediated endocytosis and intralesional, intraperitoneal or intramuscular injection. There are several protocols for human gene therapy which have been approved for use by the Recombinant DNA Advisory Committee (RAC) which conform to a general protocol of target cell infection and administration of transfected cells (see for example, Blaese, R. M., et al., 1990; Anderson, W. F., 1992; Culver, K. W. et al., 1991). In addition, U.S. Pat. No. 5,399,346 (Anderson, W. F. et al., Mar. 21, 1995, U.S. Ser. No. 220,175) describes procedures for retroviral gene transfer. The contents of these support references are incorporated in their entirety into the subject application. Retroviral-mediated gene transfer requires target cells which are undergoing cell division in order to achieve stable integration hence, cells are collected from a subject often by removing blood or bone marrow.

Several methods have been developed over the last decade for the transduction of genes into mammalian cells for potential use in gene therapy. In addition to direct use of plasmid DNA to transfer genes, retroviruses, adenoviruses, parvoviruses, and herpesviruses have been used (Anderson et al., 1995; Mulligan, 1993; The contents of which are incorporated in their entirety into the subject application).

Alternatively, the transgenic nonhuman mammal may be transfected with a suitable vector which encodes an appropriate ribozyme or antisense molecule. See for example, Leder and Stewart, U.S. Pat. No. 4,736,866 for methods for the production of a transgenic mouse. Such antisense vector may be used as a gene therapy in humans to inhibit the expression of a gene in the forebrain.

This invention is illustrated in the Experimental Details section which follows. These sections are set forth to aid in an understanding of the invention but are not intended to, and should not be construed to, limit in any way the invention as set forth in the claims which follow thereafter.

### EXPERIMENTAL DETAILS

### Example 1

Genetic and Pharmacological Evidence for a Novel, Intermediate Phase of Long-Term Potentiation (I-LTP)Suppressed by Calcineurin

To investigate the role of phosphatases in synaptic plasticity using genetic approaches, we generated transgenic mice that overexpress a truncated form of calcineurin under the control of the CaMKII( promoter. Mice expressing this transgene show increased calcium-dependent phosphatase activity in hippocampus. Physiological studies of these mice and parallel pharmacological experiments in wild-type mice reveal a novel, intermediate phase of LTP (I-LTP) in the CA1 region of hippocampus. This intermediate phase differs from E-LTP in requiring multiple trains for induction, and in being dependent on PKA. It differs from L-LTP in not requiring new protein synthesis. These data suggest that calcineurin acts as an inhibitory constraint on I-LTP that is relieved by PKA. This inhibitory constraint acts as a gate to regulate the synaptic induction of L-LTP.

To examine the role of specific phosphatases in synaptic plasticity, we have turned to a genetic approach. We have focused our initial efforts on calcineurin (PP2B), because this enzyme is thought to be the first step in a phosphatase cascade initiated by Ca²⁽ influx through the NMDA receptor. Pharmacological inhibitors of calcineurin block NMDA-receptor-dependent LTD (Mulkey et al., 1994), and have been reported to enhance LTP (Wang and Kelly, 1996; but see Wang and Stelzer, 1994; Wang and Kelly, 1997; Lu et al., 1996).

Calcineurin is a calcium-sensitive serine/threonine phosphatase that is present at high levels in the hippocampus and is enriched at synapses (Kuno et al., 1992). Once activated, calcineurin can act both directly and indirectly on protein substrates (for review, see Yagel, 1997). First, it can dephosphorylate target proteins directly and thereby regulate specific cellular functions. Second, it can modulate an even larger variety of substrates indirectly by its ability to dephosphorylate inhibitor-1. Inhibitor-1 is a low molecular weight protein that, when phosphorylated, inhibits the function of protein phosphatase-1 (PP1). Dephosphorylation of inhibitor-1 by calcineurin activates PP1 and leads to the dephosphorylation of a large and independent set of target proteins.

One interesting feature of the regulatory actions of calcineurin comes from its interactions with the cAMP-dependent protein kinase, PKA. Calcineurin inhibits the action of inhibitor-1 by dephosphorylating the site on inhibitor-1 phosphorylated by PKA. Indeed, calcineurin and PKA antagonistically regulate the function of several proteins, including NMDA and GluR6 glutamate receptors (Tong et al., 1995; Raman et al., 1996; Traynelis and Wahl, 1997) and the transcription factor CREB (Schwaninger et al., 1995; Bito et al., 1996). Further, calcineurin also inhibits a novel isoform of adenylyl cyclase (Paterson et al., 1995).

The interactions of PKA and calcineurin are of particular interest in the context of LTP. Based on the requirement for macromolecular synthesis, LTP can be divided into at least two components: an early component (E-LTP) and a late component (L-LTP). Delivery of a single 100 Hz train lasting one second to the Schaffer collateral-CA1 pyramidal cell (SC-CA1) synapse elicits E-LTP, a relatively short-lived and weak enhancement of synaptic transmission that does not require protein- and RNA-synthesis and is not dependent on PKA (Huang et al., 1996; Roberson et al., 1996). By contrast, administration of three or four trains of 100 Hz, elicit L-LTP, a more robust and stable form of LTP lasting many hours that is dependent on the activation of PKA as well as the synthesis of both RNA and protein (Huang et al., 1996; Roberson et al., 1996; Abel et al., 1997). Recent experiments with inhibitors of phosphatases suggest that one role of PKA in LTP in area CA1 may be to suppress the actions of PP1 or PP2A (Blitzer et al., 1995; Thomas et al., 1996). In particular, Blitzer et al. (1995) found that when LTP in area CA1 is induced by strong stimuli it can be blocked by inhibitors of PKA. However, this effect of PKA inhibitors was removed by preincubation of slices with PP1/PP2A inhibitors. This led Blitzer et al. to suggest that under certain circumstances, PKA may “gate” LTP by suppressing a phosphatase cascade.

To examine further the role of phosphatases in synaptic plasticity and in memory storage, as well as to determine more precisely the interplay between PKA and phosphatases in the regulation of LTP, we have overexpressed in the mouse forebrain a truncated form of calcineurin Aa. Overexpression of this transgene results in an approximately 75% increase in phosphatase activity in hippocampus. Using these mice, we have addressed two questions: (1) What is the role of calcineurin in the expression of the various phases of LTP? (2) Does PKA modulate the action exerted by calcineurin on each of these phases?

We provide both genetic and pharmacological evidence consistent with the “gating” model for the actions of PKA in LTP. In addition, data presented in this paper extend this model by demonstrating that the PKA “gate” represents an intermediate phase of LTP (I-LTP). This intermediate phase is induced by multiple trains and suppressed by calcineurin. It differs from E-LTP in requiring a much stronger stimulus, the activation of PKA and the suppression of calcineurin. The intermediate phase differs from L-LTP in not requiring protein synthesis. Our data further suggest that this constraint on I-LTP imposed by calcineurin can be relieved by activation of PKA, and that this relief is required for the full expression of L-LTP. Thus, the overexpression of calcineurin suppresses both I-LTP and L-LTP. The behavioral results detailed in the accompanying article (Mansuy et al., 1998) suggest that this distinct gating function, mediated by calcineurin, is important behaviorally and suppresses long-term memory formation.

Results

Generation of Transgenic Mice Overexpressing a Truncated Form of Calcineurin

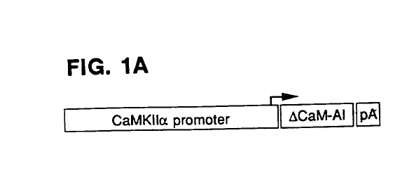

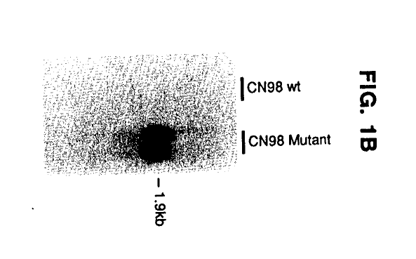

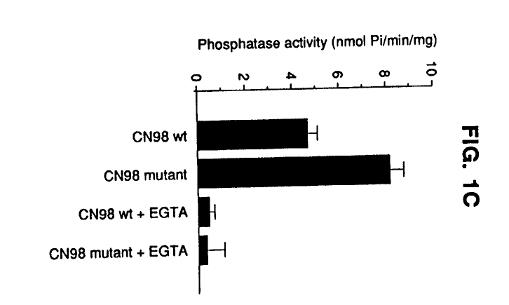

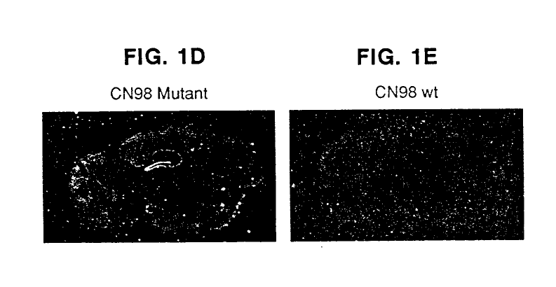

To increase the levels of calcineurin in the forebrain of transgenic mice, we expressed a deletion mutant of the catalytic subunit Aα (ΔCaM-AI) of murine calcineurin (O'Keefe et al., 1992) under the control of the CaMKIIa promoter (Line CN98, FIG. 1A; Mayford et al., 1997). The calcineurin mutant ΔCaM-AI is a fragment of the catalytic Aα subunit which lacks the autoinhibitory domain and a portion of the calmodulin binding domain, but retains the calcineurin B-binding domain (O'Keefe et al. 1992; Parsons et al., 1994). This deletion weakens the enzyme's calcium requirement. Although this construct shows some Ca²⁽ independent activity when expressed in Jurkat cells (O'Keefe et al., 1992), we find that it requires calcium for activation in hippocampal neurons (FIG. 1C).

Calcineurin Overexpression is Primarily Restricted to the Hippocampus In CN98 Mutant Mice

Northern blot analyses performed on adult CN98 mutant mouse forebrain revealed the expression of a 1.9 kb transcript corresponding to the transgene mRNA (FIG. 1B). The brain distribution of this mRNA was determined by in situ hybridization using a radiolabeled oligonucleotide specific for the transgene. The mRNA was detected in forebrain, throughout the hippocampus and dentate gyrus (FIG. 1D). No signal was detected in wild-type littermates (FIG. 1D)

To determine if the transgene mRNA was translated into a functional protein, we measured phosphatase activity in homogenates of hippocampus in the presence of okadaic acid (FIG. 1C). In the extracts of transgenic hippocampi, there was an increase of 76%±12% in phosphatase activity compared to wild-type. In the presence of the calcium chelator EGTA, the phosphatase activity in both CN98 mutant and wild-type hippocampal extracts was virtually abolished (FIG. 1C). Thus, CN98 mutant mice have significantly increased levels of calcium-stimulated phosphatase activity in hippocampus.

Basal Synaptic Transmission Is Not Altered in Mice Overexpressing Calcineurin

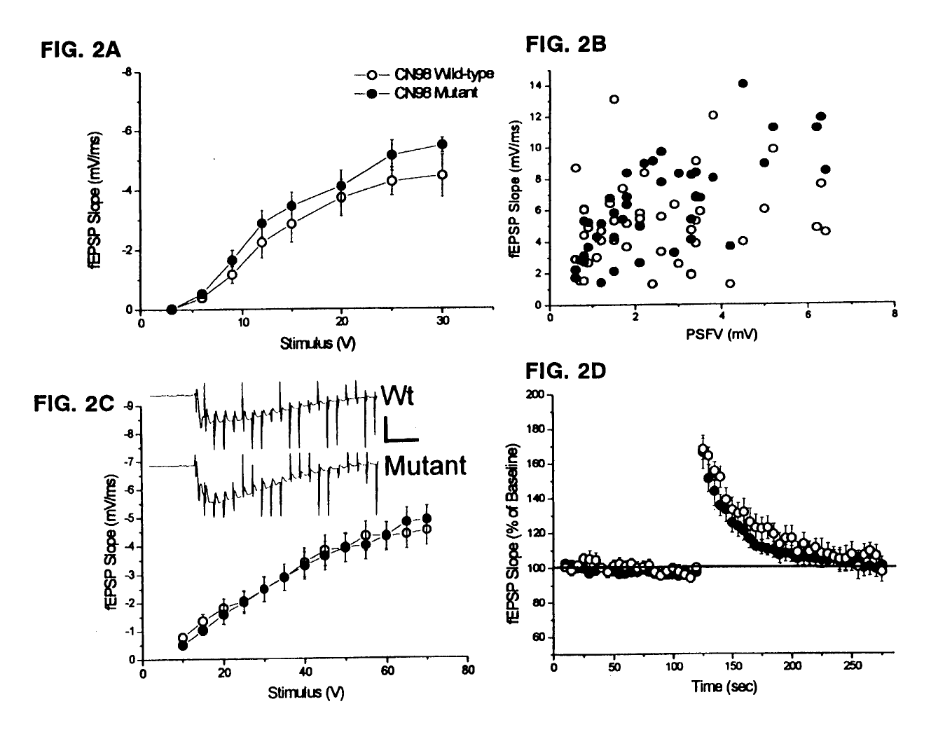

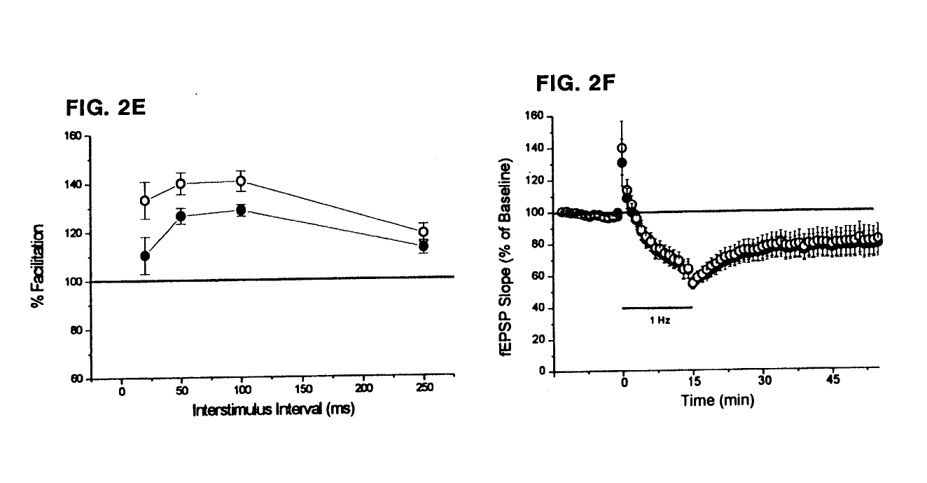

Studies with pharmacological inhibitors have suggested that endogenous phosphatases may regulate the basal level of synaptic transmission at the SC-CA1 synapse (Figurov et al., 1993). In CN98 mice however, we found no difference in basal synaptic transmission. Stimulus-response curves obtained from CN98 wild-type and mutant mice were not significantly different (FIG. 2A), and the slope of a fEPSP elicited by a given presynaptic fiber volley did not differ between wild-type and mutant (FIG. 2B).

In addition to basal transmission mediated primarily by non-NMDA ionotropic glutamate receptors, previous studies have demonstrated that activation of calcineurin can subtly desensitize NMDA receptor function (Tong et al., 1995; Raman et al., 1996). To determine whether overexpression of calcineurin altered NMDA-mediated synaptic transmission in CN98 mice, we measured NMDA-mediated synaptic potentials in the presence of 10 (M 6,7-dinitroquinoxaline-2,3-dione (DNQX) and reduced Mg²⁺ (50 μM). Under these conditions, field potentials exhibited slower kinetics than in the absence of DNQX, and were abolished by 50 (M DL-AP5, indicating that they were mediated by NMDA receptors.

Stimulus-response curves generated for both CN98 mutant and wild-type animals under these conditions were not significantly different, suggesting that overexpression of calcineurin does not alter the function of the NMDA receptor (FIG. 2C). In addition, under these conditions NMDA-mediated synaptic responses in mutant slices followed a 100 Hz, one second tetanus (FIG. 2C), as well as multiple 100 Hz trains in a qualitatively similar manner to wild-types.

Because in the CN98 mutant mice the transgene is expressed in both CA1 and CA3 pyramidal cells, we next evaluated presynaptic function. We began by assessing post-tetanic potentiation (PTP, for review, see Zucker, 1989), a short-term form of presynaptic plasticity elicited by a high frequency tetanus (1 second, 100 Hz). In the presence of DL-AP5 (50 mM) to block NMDA-receptors, administration of a single 100 Hz tetanus resulted in enhancement of transmission that decayed to baseline within 2-3 minutes. As evident in FIG. 2D, there was no difference in the peak PTP elicited between wild-type and mutant mice (160%±5% peak potentiation in wild-type, 11 slices, 5 mice; 163%±11% peak potentiation in CN98 mutant, 11 slices, 4 mice) These results suggest that overexpression of calcineurin does not markedly affect the ability of the SC-CA1 synapse to respond to high frequency rates of stimulation.

As a second measure of presynaptic function, we examined paired-pulse facilitation (PPF). PPF is a more transient form of presynaptic plasticity in which the second of two closely-spaced stimuli elicits enhanced transmitter release due to residual calcium in the presynaptic terminal following the first stimulus (Zucker, 1989). We found that over intervals of 20-250 msec PPF was significantly reduced in CN98 mutant compared to wild-type mice (15 slices, 5 mice CN98 wild-type; 14 slices, 5 mice CN98 mutant; for 20, 50, and 100 ms interstimulus intervals p<0.05 for CN98 wild-type versus mutant; FIG. 2E). In total, these data show that although overexpressing calcineurin produces no gross deficits in synaptic transmission, it does produce a clear alteration in one form of acute presynaptic plasticity.

Overexpression of Calcineurin Does Not Affect the Expression of LTD at theSC-CA1 Pyramidal Cell Synapse

To begin to study the roles of calcineurin in synaptic plasticity, we studied LTD at the SC-CA1 synapse. As has previously been reported, LFS did not elicit LTD in adult animals (Bear and Abraham, 1996). We therefore repeated these studies in slices from young mice (3-4 weeks old) where LTD is more robust. As shown in FIG. 2F, although LTD was much more robust in these younger animals, there was no difference detectable between CN98 wild-type and mutant animals (fEPSP slope percent of baseline 30 minutes after the end of 15 minutes of 1 Hz stimulation: CN98 wild-type 79 (8%, 2 animals, 4 slices; CN98 mutant 76 (7%, 4 animals, 7 slices). One possibility consistent with these data is that calcineurin may already be present at saturating concentrations, particularly since calcineurin is one of the most abundant proteins in brain (Yakel, 1997). If calcineurin were present in saturating concentrations, one would predict that further overexpression of calcineurin would not affect processes such as LTD that are likely mediated by activation of the phosphatase. However, overexpression might alter synaptic processes such as LTP where the suppression of phosphatase activity is thought to be required.

Overexpression of Calcineurin Diminishes LTP Induced by Multiple High-Frequency Trains but not a Single Train

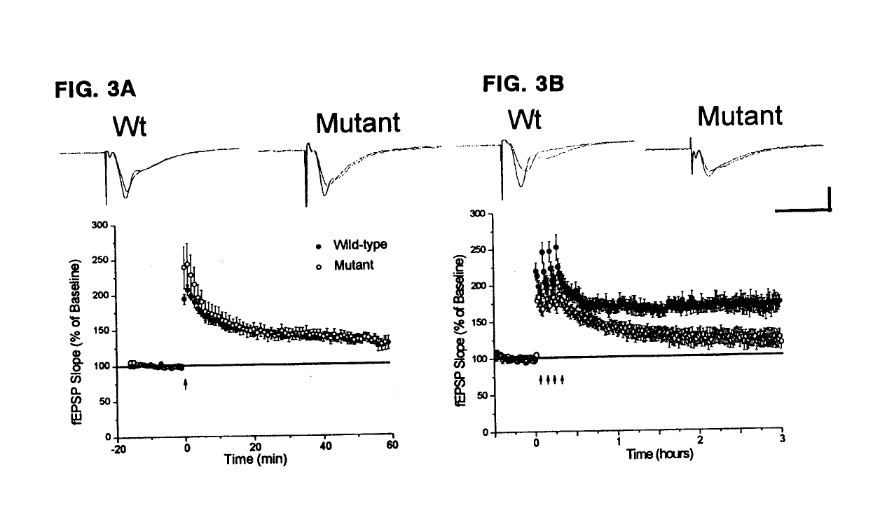

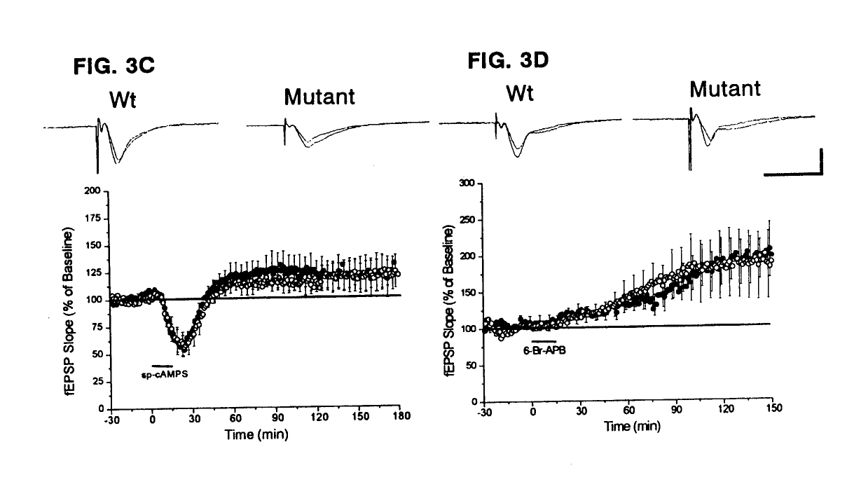

Next, we studied LTP induced by single or multiple one-second high frequency (100 Hz) trains in wild-type and CN98 mutant mouse hippocampal slices. Administration of a single train at 100 Hz elicited a transient form of LTP that was comparable in mutant and wild-type slices at one hour post-tetanus, even though immediately after the tetanus LTP was slightly reduced in CN98 mutants (CN98 mutant: 129±10% of baseline at 1 hr, 9 slices, 5 mice; CN98 wild-type: 130±6% of baseline at 1 hr, 7 slices, 4 mice; FIG. 3A). By contrast, administration of four 100 Hz trains separated by 5 minutes elicited robust, nondecremental LTP in wild-type hippocampal slices, but produced a greatly reduced LTP in mutant mice (CN98 wild-type: 169±8% of baseline at 1 hr after stimulus, 173±8% at 3 hr, 7 slices, 7 mice; CN98 mutant: 139±9% of baseline at 1 hr after stimulus, 118±10% at 3 hr, 8 slices, 7 mice; FIG. 3B). This defect in the CN98 mutant animals was visible immediately after the four tetani were administered (p<0.05 at 1 minute after the last tetanus).

Overexpression of Calcineurin Does Not Affect Chemically-Induced L-LTP

The finding that LTP induced by four trains but not a single train is reduced in CN98 mutant mice suggests that overexpression of calcineurin may suppress the late phase of LTP. Is this reduction due to a direct effect on downstream components of L-LTP, or is it due to a failure to fully initiate L-LTP? To explore this question we examined L-LTP evoked by pharmacological activation of the PKA pathway, which bypasses tetanic stimulation in area CA1. In wild-type slices, application of agonists of D1/D5 dopamine receptors or the PKA agonist Sp-cAMPS, results in a slow-onset potentiation of synaptic transmission that is sensitive to protein and RNA-synthesis inhibitors, and mutually occlusive with L-LTP elicited by multiple high frequency trains (Huang et al., 1996; Bolshakov et al., 1997). If overexpression of calcineurin directly affects the machinery necessary to produce the late phase, pharmacologically-induced L-LTP, that bypasses E- and I-LTP, would be impaired in CN98-mutant mice, as is the case with the late phase deficit in tPA-knockout mice (Huang et al., 1996).

We tested the ability of both the D1/D5 receptor agonist 6-Br-APB (100 mM) and the PKA activator Sp-cAMPS (100 mM) to elicit slow-onset potentiation at the SC-CA1 synapse in CN98 mice. As shown in FIG. 3C and D, application of 6-Br-APB and Sp-cAMPS elicited a slowly-developing increase in synaptic transmission in CN98 mutant mice that was indistinguishable from that seen in wild-type mice (CN98 mutant: 181±41% of baseline at 3 hr after 6-Br-APB application, 5 slices, 5 mice; CN98 wild-type: 204±40% of baseline at 3 hr after 6-Br-APB application, 3 slices, 3 mice; CN98 mutant: 122±17% of baseline at 3 hr after Sp-cAMPS application, 6 slices, 6 mice; wild-type: 124±13% of baseline at 3 hr after Sp-cAMPS application, 7 slices, 6 mice).

Multiple Trains Elicit Two Distinct PKA Dependent Phases of LTP: One Dependent and the Other Independent of Protein Synthesis

In contrast to wild-type hippocampal slices where LTP induced by a single train is much weaker than that induced by four trains, in slices from CN98 mutants the magnitude of LTP that follows one train and four train protocols were similar. Indeed, the LTP following four trains in CN98 mutants is quite similar to that evoked by four trains in wild-type hippocampal slices incubated with inhibitors of PKA (for review see Huang et al., 1996), as well as to L-LTP in hippocampal slices from mice expressing a dominant negative form of PKA (Abel et al., 1997). This would make it appear as if the PKA system is defective or reduced in its effectiveness in the mutant mice. Yet L-LTP induced by pharmacological activation of the cAMP cascade was not dramatically impaired in the mutant mice. How then do PKA and calcineurin interact?

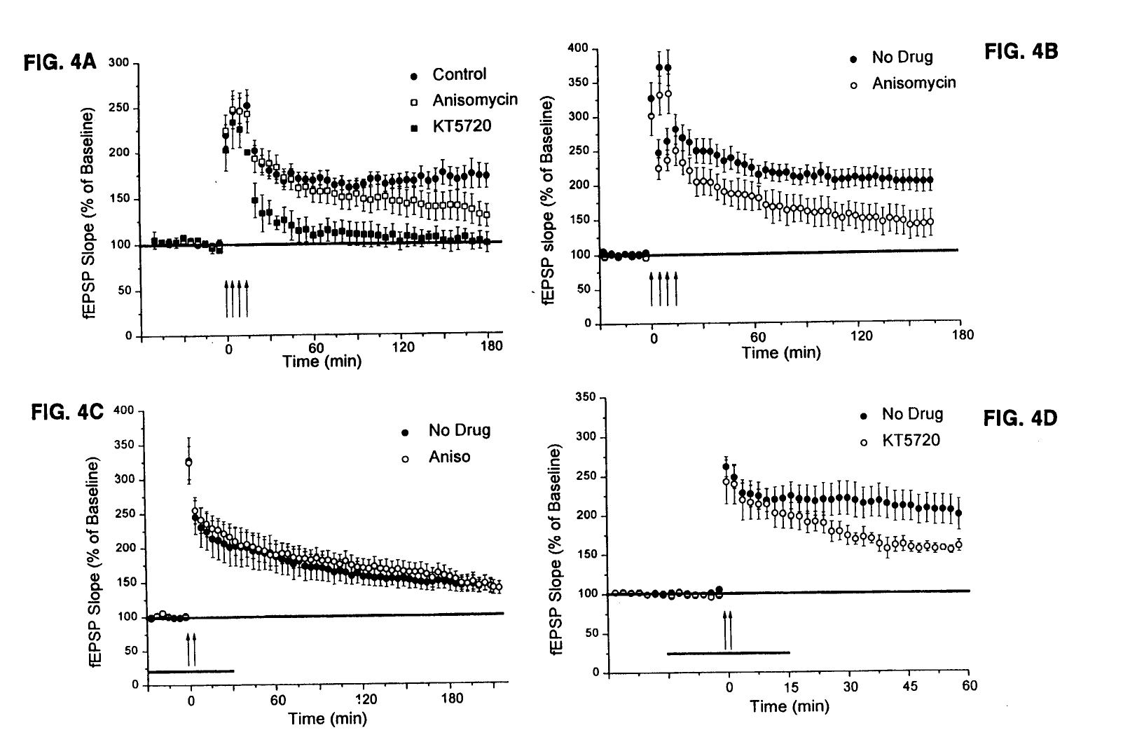

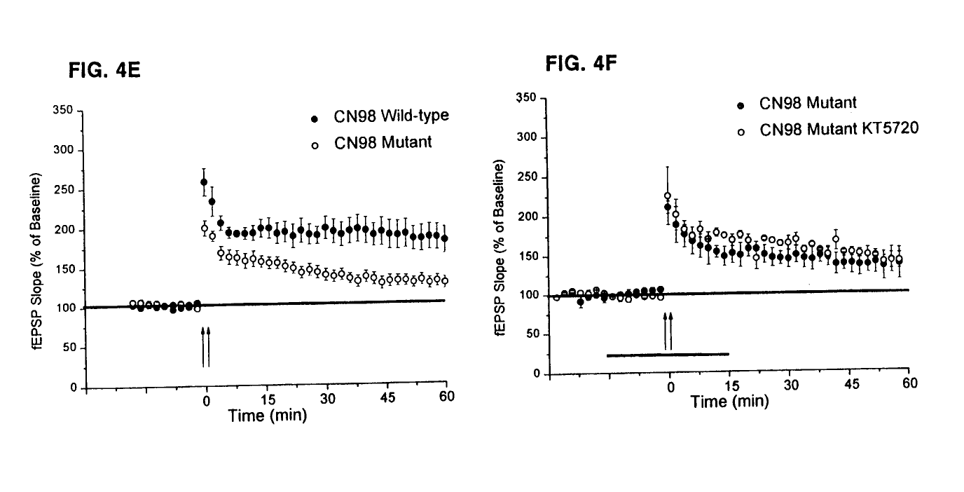

One clue to the possible interaction of calcineurin with the PKA system in regulating LTP comes from the work of Blitzer et al. (1995) and Abel et al. (1997) showing that application of inhibitors of PP1 and PP2A removes the ability of PKA inhibitors to block LTP after a strong stimulus, suggesting that one role of PKA in LTP in area CA1 may be to inhibit the actions of phosphatases that are activated by tetanus. This would suggest that PKA may serve a double function. First, it can activate the late phase directly (FIGS. 3C,D). Second, PKA has an earlier function in turning off an opposing phosphatase cascade. Consistent with this hypothesis, LTP generated by multiple 100_Hz trains in rat hippocampal slices (Huang et al., 1996), as well as mouse hippocampal slices (FIG. 4A) decays more rapidly in the presence of PKA inhibitors such as Rp-cAMPS or KT5720 than in the presence of the protein synthesis inhibitor anisomycin (Blitzer et al., 1995; Huang et al., 1996).

To examine further the possibility that there are two independent phases both dependent on PKA, we reanalyzed the effects of anisomycin on LTP in mouse hippocampal slices. The concentrations of anisomycin used here (30 μM) are sufficient to completely block protein synthesis in area CA1 (Stanton and Sarvey, 1984; Osten et al., 1996). Nonetheless, the difference in timecourse of inhibition by anisomycin and PKA inhibitors could be due to pharmacokinetic properties of these drugs. However, even in experiments where anisomycin (30 μM) was present in the bath for one full hour prior to tetanus (compared to the 20 minute pretreatment with the PKA inhibitor KT5720, 1 mM), the PKA inhibitor still elicited a much more rapid decay of LTP induced by four 100 Hz trains than anisomycin (FIGS. 4A,B) . This difference in timecourse between inhibitors of protein synthesis and PKA suggests that multiple trains that elicit L-LTP seem also to induce a novel intermediate phase of LTP that requires PKA but does not require protein synthesis.

A Novel PKA Dependent Intermediate Phase Can Also Be Isolated by Varying the Number of Stimulus Trains

To further isolate this intermediate phase, we varied the number of tetanic trains of stimulation. One of the characteristics that distinguishes E-LTP from L-LTP is that weak stimuli such as a single 100 Hz train elicit E-LTP but not L-LTP. In contrast, to reliably induce L-LTP, 3-4 repeated 100 Hz trains are required. We therefore sought to determine if an intermediate phase of LTP could also be distinguished from these phases based on the strength of stimulus required. We elicited LTP with two 100 Hz trains spaced by 20 seconds. This protocol elicited LTP that, on average, was more robust than that elicited by one 100 Hz train, but less maintained than that elicited by four trains (FIG. 4C). In contrast to LTP elicited by a single 100 Hz train which is not affected by inhibitors of PKA (Huang et al., 1996), LTP elicited by two trains was reduced by the PKA inhibitor KT5720 (no drug: 206±23% of baseline at 1 hr, 5 slices, 5 mice; 1 mM KT5720: 153±5% of baseline at 1 hr, 5 slices, 4 mice; p<0.05; FIG. 4D). However, unlike L-LTP, the LTP elicited by two trains was completely insensitive to preincubation with the protein synthesis inhibitor anisomycin, even at time points where LTP induced by four trains is reduced by anisomycin (FIG. 4C). These experiments reveal a novel intermediate phase of LTP (I-LTP) exists that requires 1) a stronger stimulus than E-LTP, and 2) the activation of PKA. But unlike L-LTP, this intermediate phase does not require protein synthesis.

Genetic Evidence for an Interaction Between PKA and Phosphatasesin Regulating a Novel Intermediate Phase of LTP (I-LTP)

The data from Blitzer et al. (1995) and Thomas et al. (1996) suggest that the protein synthesis-independent role of PKA in LTP is to suppress the activity of PP1 or PP2A, perhaps through phosphorylation of inhibitor-1. Since the phosphorylation site of inhibitor-1 is dephosphorylated by calcineurin, PKA and calcineurin can antagonistically regulate the function of PP1 and thereby perhaps regulate the level of synaptic output. Indeed, one train LTP, which is independent of PKA, was not decreased in CN98 mutant mice, while PKA-dependent four train LTP was. To examine this further, we compared CN98 wild-type and mutant mice by examining LTP induced by two trains, which we have shown recruits the intermediate phase without significantly recruiting the late phase. Consistent with the idea that the intermediate phase of LTP is antagonistically regulated by PKA and calcineurin, LTP elicited by two trains in mutant mice was markedly impaired (CN98 mutant: 127±7% of baseline at 1 hr, 12 slices, 7 mice; CN98 wild-type: 182±17% of baseline at 1 hr, 8 slices, 4 mice; p<0.05; FIG. 4E). Moreover, the LTP that remained in the mutant mice was insensitive to PKA inhibition, suggesting further that the function of PKA in the intermediate phase is to relieve the actions of calcineurin (FIG. 4F).

Overexpression of the Calcineurin Transgene Restricted to Postsynaptic CA1 Pyramidal Cells is Sufficient to Interfere with the Intermediate Phase of LTP

The phenotype of CN98 mutant mice suggests that calcineurin suppresses an intermediate phase of LTP. However, because the calcineurin construct in these mice is expressed both pre- and postsynaptically, we cannot tell from these experiments alone where calcineurin is eliciting its action. In addition, subtle alterations in presynaptic function, such as those observed in PPF in these mice could contribute to the phenotype. To investigate this possibility, as well as to verify that the deficit in I-LTP seen is not due to an insertion site effect, we analyzed two additional lines of mice which express the calcineurin transgene in a more spatially restricted manner in hippocampus. The two lines we tested, (Tet-CN279 and Tet-CN273 ), had the further advantage that the expression of the calcineurin transgene is regulated by the tetracycline-controlled transactivator (tTA) system (see Example 2 hereinbelow, Mansuy et al., 1998, for details of generation and characterization of these two lines). In contrast to line CN98, in which the transgene is strongly expressed both in CA3 and CA1 pyramidal cells, in lines Tet-CN279 and Tet-CN273 the transgene is expressed much more strongly in the CA1 postsynaptic pyramidal cells than in the CA3 presynaptic pyramidal cells at the SC-CA1 synapse.

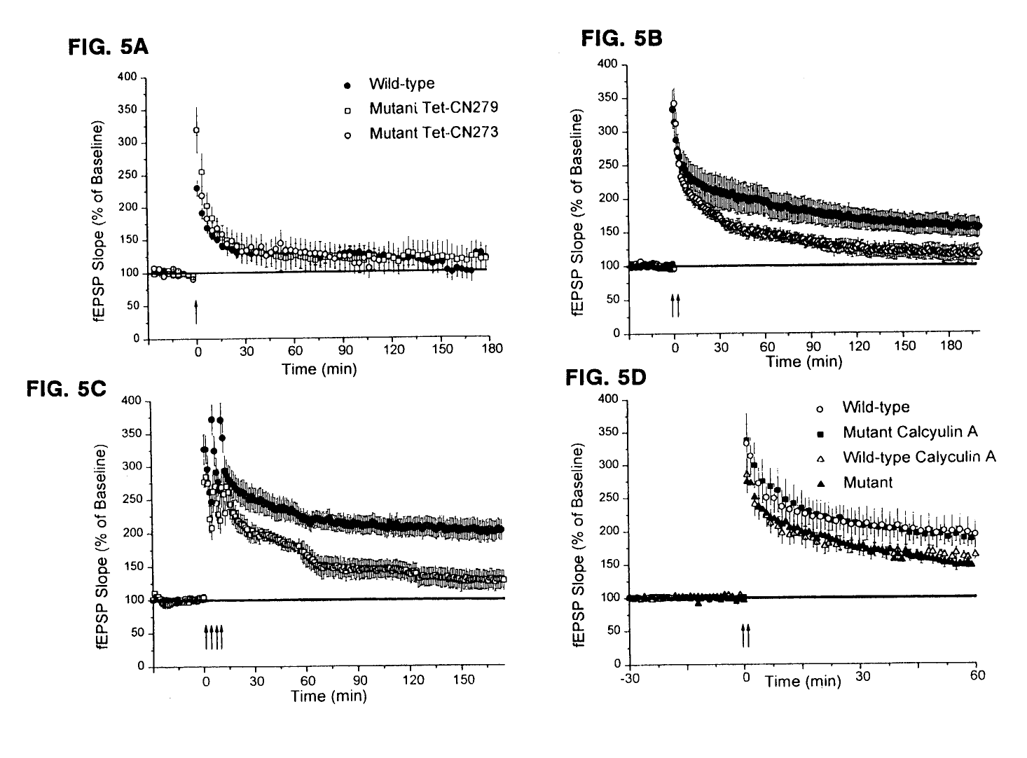

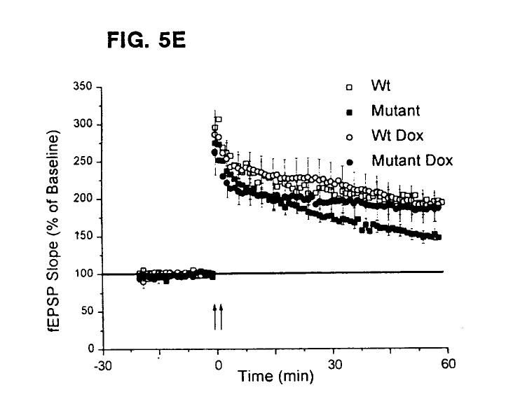

We first determined the effects of overexpression of the transgene in CA1 pyramidal cells on LTP by comparing slices from Tet-CN273 and Tet-CN279 on LTP elicited by one and two trains, and LTP induced by four 100 Hz trains in Tet-CN279 mice. Consistent with the results in the CN98 line, overexpression of the calcineurin transgene under the Tet-system had no effect on LTP induced by a single train, but reduced LTP elicited by two and four trains (FIGS. 5A-E). Interestingly, in contrast to the CN98 mice, where LTP was reduced immediately after two 100 Hz trains, both Tet-CN279 and Tet-CN273 mutant mice, which also exhibit a deficit in two train at 1 hour, showed little or no deficit immediately after the tetanus. Thus, the phenotype in these lines more closely parallels the defect observed after application of PKA inhibitors to wild-type slices than does the CN98 line, and supports the idea that delineation of the intermediate phase in these mutant mice is not an artifact of reduced presynaptic function. Further, these data imply that the site of action of the phosphatase cascade is postsynaptic at the SC-CA1 synapse.

The Suppression of the Intermediate Phase of LTP by Overexpression of Calcineurin Can Be Rescued by Application of PP1 Inhibitors

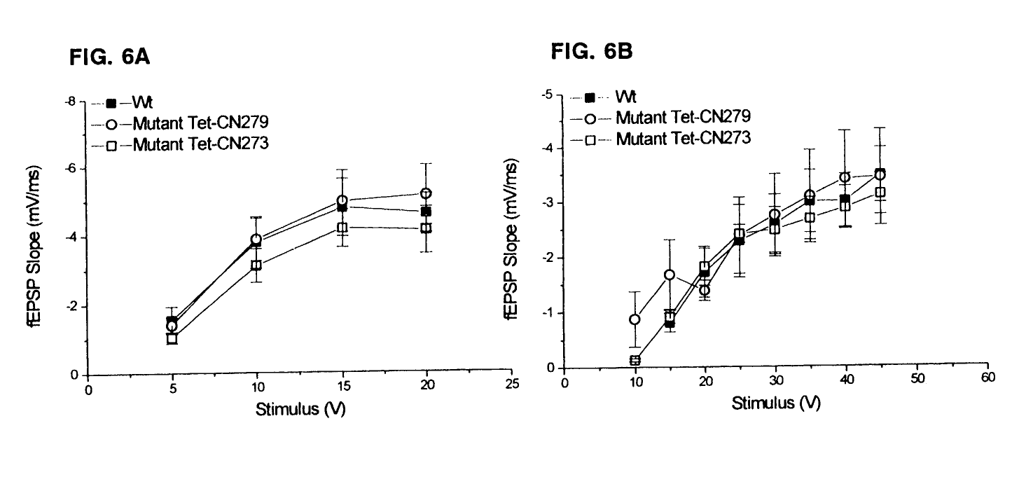

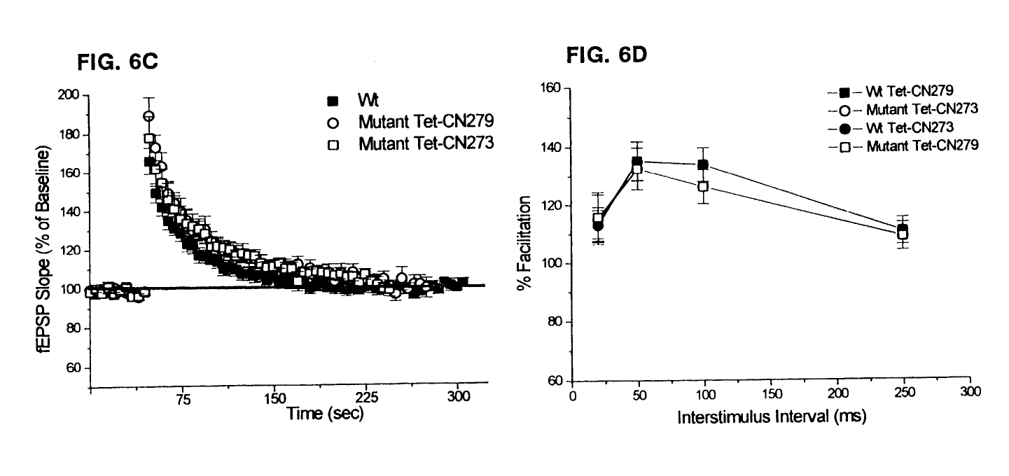

Similar to the results obtained in line CN98, we found no detectable differences in basal synaptic transmission, NMDA receptor-mediated synaptic potentials, and PTP in wild-type and mutant animals from lines Tet-CN273 and Tet-CN279 (FIG. 6A-C). In contrast to the results in the CN98 line, however, we saw no deficits in PPF in line Tet-CN279 or Tet-CN273, consistent with weak or absent expression of the transgene presynaptically (FIG. 6D).

Because PKA can regulate PP1 function through phosphorylation of inhibitor-1, a site dephosphorylated by calcineurin, preincubation of hippocampal slices from mice overexpressing calcineurin with a PP1 inhibitor should rescue LTP if this cascade is utilized. To test this hypothesis, we pretreated slices from Tet-CN279 mutant and wild-type mice for 30 minutes with 750 nM calyculin A, after which LTP was induced with two 100 Hz trains. Consistent with the hypothesis that overexpressed calcineurin is suppressing LTP by regulating the activity of PP1, pretreatment of slices with calyculin A resulted in LTP in mutant mice that was indistinguishable from that seen in wild-type (FIG. 5D).

Regulated-Overexpression of the Calcineurin Transgene Suggests that the Deficit in I-LTP Is Not Due to Developmental Effects of the Transgene in Hippocampus

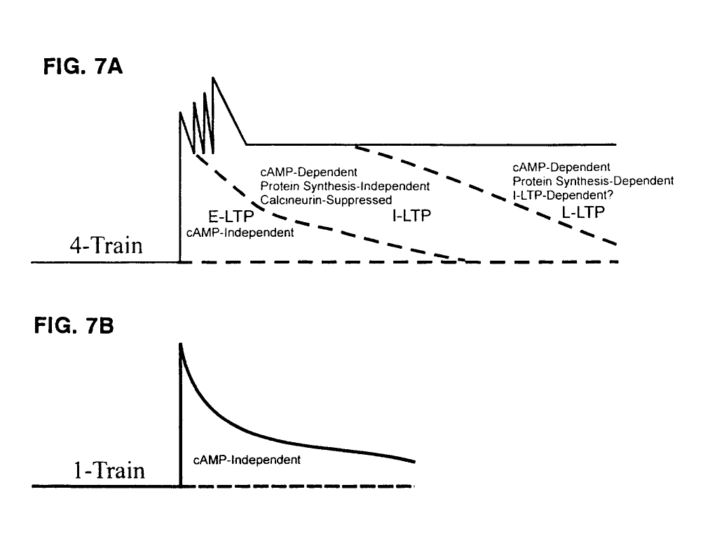

The tTA system allows regulation of transgene expression, providing a means to address whether the phenotype observed in mice overexpressing calcineurin reflected a consequence of the transgene on development of the nervous system or represented an acute effect of the transgene on synaptic plasticity. In the absence of doxycycline, the transgene is expressed in the Tet-CN279 mice (Mansuy et al., 1998). However, when doxycycline (1 mg/ml) is administered in the animal's water supply, or in the ACSF (1 ng/ml) during electrophysiological experiments, expression is suppressed (Mansuy et al., 1998). We therefore compared LTP induced by two trains in Tet-CN279 mutant and wild-type mice on or off doxycycline. In wild-type mice either on or off doxycycline, stimulation with two trains resulted in robust LTP indistinguishable from that elicited in CN98 wild-type mice (Tet-CN279 Wt: 195 +13% of baseline at 1 hr, 7 slices, 6 mice; Tet-CN279 Wt on doxycycline: 191±18% of baseline at 1 hr, 12 slices, 7 mice; FIG. 5E). In Tet-CN279 mutant mice off doxycycline, the response to two trains was significantly lower than that in wild-type one hour after the tetanus, and was completely reversed by doxycycline pretreatment (Tet-CN279 mutant: 147±8% of baseline at 1 hr, 15 slices, 9 mice; Tet-CN279 mutant on doxycycline: 184±18% of baseline at 1 hr, 8 slices, 5 mice; p<0.01 for Tet-CN279 mutant versus Tet-CN279 wild-type, FIG. 7B). These results suggest that the calcineurin transgene produces its effect on the intermediate phase of LTP postsynaptically in the adult animal, and its effect is not attributable to a developmental consequence of the transgene.

Discussion

Using a genetic approach to study the role of phosphatases in synaptic plasticity, we focused on calcineurin because it appears to function in the hippocampus as a first step in a calcium-dependent cascade of phosphatases. To limit the expression of the transgene to forebrain, and reduce the likelihood that the phenotype produced is a result of the presence of the transgene during development, we overexpressed calcineurin using the CaMKIIa promoter. To control further for a developmental role of the transgene, as well as to control for insertion-site dependent effects, we also studied two other lines of mice (Tet-CN279 , Tet-CN273 ) in which the phenotype exhibited by CN98 mice can be reproduced and reversed by suppression of the expression of the transgene using a regulatable transactivator (see Mansuy et al., 1998). With these lines we show that the expression of calcineurin essentially limited to the CA1 neurons within the hippocampus selectively interferes with a novel phase of LTP that we isolated independently by pharmacological and physiological means. Moreover this phenotype in mice overexpressing calcineurin is due to the expression of the transgene in the adult animal.

An Intermediate Component of LTP, I-LTP, Modulated by Calcineurin and PKA

Converging lines of evidence, both from pharmacological studies as well as genetic studies with calcineurin overexpressing mice suggest that an intermediate phase of LTP exists, and that this phase is suppressed by calcineurin. This suggestion is based on several findings (FIG. 7). First, E-LTP and I-LTP differ in three ways: 1) E-LTP is independent of PKA, whereas I-LTP is dependent on PKA. 2) I-LTP, but not E-LTP, is inhibited by overexpression of calcineurin. Finally, 3) I-LTP requires a stronger stimulus for initiation than E-LTP.

Second, I-LTP can be distinguished from L-LTP by two ways: 1) whereas both I-LTP and L-LTP are dependent on PKA, only L-LTP is dependent on protein synthesis; and 2) while I-LTP could not be generated in mice overexpressing calcineurin, pharmacologically induced slow-onset potentiation, which is thought to utilize the same mechanisms as tetanically-induced L-LTP can still be generated.

Previous studies have suggested that an early, apparently protein synthesis-independent component of LTP requires PKA. For example, while LTP induced by multiple trains is rapidly inhibited by blockers of PKA, it was inhibited more slowly by blockers of protein synthesis (Blitzer et al., 1995; Huang et al., 1996). Further, Thomas et al. (1996) found that activation of β-adrenergic receptors by isoproterenol enables subthreshold stimuli to elicit robust enhancement of synaptic transmission at the SC-CA1 synapse in a PKA-dependent manner. These effects have been interpreted to reflect a PKA-mediated suppression of phosphatase activity, based on the findings that phosphatase inhibitors prevented PKA inhibitors from blocking LTP (Blitzer et al, 1995) and mimicked the effects of activating PKA (Thomas et al., 1996). While these studies suggest that a role of PKA in LTP is to suppress phosphatase activity, they cannot exclude an alternative explanation, that the phosphatase inhibitors enhanced the actions of residual, incompletely antagonized PKA. Moreover, although calcineurin was proposed to participate in suppressing LTP, the inhibitors used in these studies are ineffective in blocking calcineurin, making it unclear whether calcineurin is important in regulating LTP. In fact, application of inhibitors of calcineurin to hippocampal slices has yielded contradictory results, with some studies reporting no effect (Mulkey et al., 1994; Muller et al., 1995) or enhancement (Wang and Kelly, 1996) of LTP, while other studies report blockade of LTP (Wang and Stelzer, 1994; Wang and Kelly, 1997; Lu et al., 1996). Using a genetic approach, we demonstrate that PKA suppresses a phosphatase cascade by showing that overexpression of calcineurin removes the PKA-dependent component of LTP. Because this suppression is rescued by the PP1/PP2A inhibitor calyculin A, these data are also consistent with the proposed model that calcineurin and PKA interact at the level of inhibitor-1, a molecule that controls that activity of PP1.

We would emphasize that although I-LTP and E-LTP differ in several ways, I-LTP very likely also shares a number of mechanisms in common with E-LTP. For example, the suppression of phosphatase activity by PKA during I-LTP, a suppression which requires a stronger stimulus than the one 100 Hz train necessary to produce E-LTP, may simply act to allow a more robust utilization of mechanisms recruited for E-LTP. In addition, while there is a temporal distinction between I-LTP, E-LTP and L-LTP in response to repeated high frequency trains, as well as a distinction in the strength of stimulus required to elicit these phases, these distinctions may become blurred under other circumstances, such as during periods in which neuromodulatory influences are recruited (Thomas et al., 1996). Indeed, the sensitivity of I-LTP to stimulus intensity explains why in a previous report overexpression of a dominant negative form of PKA had no effect on LTP elicited by two trains (Abel et al., 1997). When a stronger two train protocol was used that elicited LTP of a magnitude comparable to the present data, defective LTP in response to two trains was observed in R(AB) mutant mice.

Our evidence suggests that the intermediate phase of LTP is inhibited by overexpression of calcineurin. Whether endogenous calcineurin performs the same function remains to be determined. However, pharmacological experiments suggest that this may be the case (Wang and Kelly, 1996). Further, at present it is unclear which kinases and effectors responsible for this phase of LTP are suppressed by calcineurin. Thus, in future experiments it will be important to use other genetic manipulations, such as dominant negative constructs of calcineurin or calcineurin knockouts, as well as biochemical investigations of the activity of specific kinases in these mutants to investigate this intermediate phase further.

Interestingly, we find that several aspects of synaptic transmission thought to be mediated by calcineurin are not altered by overexpression of this enzyme. While there are several possible explanations for our results, it seems likely that a large excess of calcineurin exists in CA1 (a calcineurin reserve). Indeed, calcineurin is one of the most abundant proteins in brain (Yagel, 1997). If this hypothesis is correct, overexpression of calcineurin would only be expected to affect physiological actions that require the endogenous suppression of phosphatase activity, since overexpression would create a larger calcineurin reserve that might make it more difficult to completely inhibit phosphatase activity. Consistent with this idea, we find that overexpression of calcineurin places an inhibitory constraint on I-LTP.

PKA Is a Feed-forward Regulator of Calcium-Stimulated Kinase Activity

Calcineurin has a particularly high affinity for calcium/calmodulin. For example, it is at least an order of magnitude more sensitive to calcium/calmodulin than CaMKII. It was this feature of calcineurin which led Lisman (1994) to propose that low-level increases in calcium, induced by low frequency stimuli, would lead to synaptic depression through activation of calcineurin, while high frequency stimuli would lead to the large increases in calcium necessary to activate CaMKII and lead to LTP (Lisman, 1994). These aspects of Lisman's model have been supported by several studies (Malenka and Nicoll, 1993; Cummings et al., 1996).

The studies herein provide support for a further model. According to Lisman's model, robust LTP requires the inactivation of phosphatases. We find that the phosphatases do indeed impose an inhibitory constraint on LTP, and suggest that PKA is required to suppress phosphatase activity sufficiently to fully elicit LTP. The calcium-sensitive adenylyl cyclases are ideally suited to increase cAMP levels and thereby inhibit the phosphatases only when large increases in intracellular calcium occurs (Lisman, 1994). Indeed, activation of NMDA receptors by robust tetanization that induces LTP increases cAMP levels in CA1 through a calmodulin-dependent process (for review, see Huang et al., 1996; Roberson et al., 1996). Therefore, while calcium directly regulates the balance of kinase and phosphatase activity, the generation of cAMP by NMDA-receptor-dependent activation of calcium-sensitive adenylyl cyclases can favor kinases further by inducing a PKA-dependent inactivation of the activation of PP1 by calcineurin through phosphorylation of inhibitor-1.

Calcineurin May Act as a Shunt of Synaptically Evoked L-LTP

In an effort to determine whether the machinery required to induce L-LTP is intact in CN98 mice we tested whether we could pharmacologically elicit the late phase in a manner that bypasses tetanus. Application of activators of the PKA cascade induced a slow-onset potentiation of transmission that was normal in CN98 mutant slices. This slow-onset potentiation of transmission is thought to utilize the same machinery as four 100 Hz trains because they both are PKA and macromolecular synthesis dependent, and are mutually occlusive (Huang et al., 1996). Indeed both tetanus-induced and pharmacologically induced L-LTP are impaired in tPA^{(/(}mice in which a molecule is ablated that is predicted to be downstream from macromolecular synthesis in the generation of L-LTP (Huang et al., 1996).

As discussed above, this reduction of LTP in CN98 mutant mice overexpressing calcineurin is likely due to a shunting of the upstream kinases important for initiating L-LTP. Indeed, two recent reports are consistent with this possibility. For example, Bito et al. (1996) have reported that CREB phosphorylation in cultured hippocampal neurons is also negatively regulated by calcineurin. Thus, regulation of transcription factors thought to be necessary for long-term synaptic modifications by calcineurin may prevent the formation of L-LTP in cases in which PKA is not activated sufficiently.

Multiple Inhibitory Constraints Must be Overcome to Evoke PKA-dependent Synaptic Plasticity

Studies in Aplysia and Drosophila first revealed that the expression of learning-related synaptic plasticity is restricted by a number of inhibitory constraints that operate in different compartments within the cell, ranging from the cell membrane to the nucleus (Yin et al., 1994, 1995; Bartsch et al., 1995). For example, Bartsch et al. (1995) found that an isoform of the transcription factor CREB (CREB-2) normally suppresses the formation of long-term facilitation by a single pulse of serotonin. However, removal of this constraint by injection of antibodies or antisense oligonucleotides directed against this transcription factor allows one pulse of serotonin, which normally only elicits short-term facilitation to elicit long-term facilitation. These studies imply that to induce long-lasting enhancement of synaptic transmission, different types of inhibitory constraints need to be overcome. Our studies with calcineurin provide evidence that inhibitory constraints are also acting on plasticity in the mammalian brain. In Example 2 hereinbelow (Mansuy et al., 1998), we show that excessive activation of this inhibitory constraint interferes with memory storage.

Materials/Methods

Plasmid Construction

A cDNA encoding a truncated form of the murine calcineurin catalytic subunit Aα, ΔCaM-AI was used to construct the expression vector for the generation of CN98 mice. A 1.27 kb EcoRI fragment of DCaM-AI cDNA was made blunt-ended and subcloned into the EcoRV site of pNN265 vector. The plasmid pNN265 carries upstream from the EcoRV site, a 230 bp hybrid intron that contains an adenovirus splice donor and an immunoglobulin G splice acceptor (Choi et al., 1991) and has a SV40 polyadenylation signal downstream from the EcoRV site. The ΔCaM-AI cDNA flanked by the hybrid intron in 5′ and the poly(A) signal in 3′ was excised from pNN265 with NotI and the resulting 2.7 kb fragment was placed downstream of the 8.5 kb mouse CaMKIIα promoter including the transcriptional initiation site (Abel et al., 1997) to generate the CN98 mice (FIG. 1A). The final 11.2 kb CaMKIIα promoter-ΔCaM-AI (FIG. 1A) was excised from the vector by digestion with SfiI. Prior to microinjection, all cloning junctions were checked by DNA sequencing.

Generation and Maintenance of CN98 Transgenic Mice

The transgenic mice CN98 were generated by microinjection of the linear constructs into fertilized eggs collected from BL6/CBA F1/J superovulated females mated with BL6/CBA F1 males (Jackson Laboratories; Hogan et al., 1994). Before microinjection, the DNA fragment was gel purified then put through ELUTIP® (Schleicher and Schuell) for further purification. Microinjected eggs were kept overnight at 37° C. in 5% CO₂ and one day later, the two-cell embryos were transferred into pseudopregnant BL6/CBA F1/J females. Analysis of founder mice for integration of the transgene was performed by Southern blotting and PCR. The founder mouse was backcrossed to C57BL6 F1/J mice to generate the transgenic line CN98. The genotype of the offspring was checked by Southern blotting or PCR. Transgenic mice were maintained in the animal colony according to standard IACUC protocol.

Northern Blot Analysis

Total RNA from adult CN98 mouse forebrain was isolated by the guanidinium thiocyanate method (Chomczynski and Sacchi, 1987). RNA (10 μg) was denatured in 1 M formaldehyde, 50% formamide, 40 mM triethanolamine, 2 mM EDTA (pH 8), electrophoresed on a 1 agarose gel and transferred to a nylon membrane (GENSCREEN PLUS®, NEN®) in 0.4 N NaOH. The membrane was hybridized to a 1.1 kb [γ³²p]dCTP-labeled EcoRV-NotI fragment from pNN265. The hybridization was performed overnight at 42° C. in 50% formamide, 2× SSC, 1% SDS, 10% dextran sulfate, 0.5 mg/ml denatured salmon sperm DNA. The membrane was washed 10 min at room temperature in 2× SSC, 1% SDS then twice 15 min at 42° C. in 0.2× SSC, 1% SDS and exposed to film for three days.

In Situ Hybridization

Adult mouse brains were dissected out and rapidly embedded in Tissue-Tek medium on dry ice. Sections were fixed and hybridized as described (Abel et al., 1997) to an [α³⁵S] DATP labeled, transgene specific oligonucleotide (5′-GCAGGATCCGCTTGGGCTGCAGTTGGACCT-3′) (Seq I.D. No. 1) derived from pNN265. Slides were exposed to film for 2-3 weeks.

Phosphatase Assay

Phosphatase assays were performed according to Hubbard and Klee (1991). Briefly, mice were injected with 5 ml/kg of pentobarbital and decapitated. Hippocampi were homogenized in 2 mM EDTA (pH 8), 250 mM sucrose, 0.1% β-mercaptoethanol and centrifuged. Supernatants were diluted in 40 mM Tris-HCl (pH 8), 0.1 M NaCl, 0.4 mg/ml bovine serum albumin, 1 mM DTT, 0.45 mM okadaic acid (Buffer 1) and incubated at 30° C. for 1 min in Buffer 1 containing 1 mM of the peptide [γ³²p]-RII subunit of cyclic AMP-dependent protein kinase (PKA) and either 0.1 mM calmodulin (SIGMA®) and 0.66 mM Ca²⁽ or 0.33 mM EGTA (pH 7.5). The peptide [Ala⁹⁷]-RII (Peninsula Labs) was labeled with 0.3 mM [γ³²p] ATP (NEN®) using 4 mg catalytic subunit of PKA (FLUKA®). The reaction was stopped with 5% TCA in 0.1 M KH₂PO₄ and the enzyme activity was calculated as previously described (Klee et al., 1983) and is expressed in nmol Pi released/min/mg protein. The protein concentration was determined using the bicinchroninic acid protein assay kit (SIGMA®). All samples were performed in triplicate.

Electrophysiology

Transverse hippocampal slices were prepared as previously described (Abel et al., 1997). Mice of either sex, aged 7-18 weeks were used. Where appropriate, the experimenter was blind to animal genotype. Hippocampi were sliced (400 μm), placed in oxygenated ACSF (NaCl, 124 mM; KCl, 4.4 mM; CaCl₂, 2.5 mM; MgSO₄, 1.3 mM; NaH₂PO₄, 1 mM; glucose, 10 mM; and NaHCO₃, 26 mM), and subfused (1-2 ml/min) in an interface chamber and allowed to equilibrate for 60-90 min at 28° C. For extracellular recordings, ACSF-filled glass electrodes (1-3 MW) were positioned in the stratum radiatum of area CA1. A bipolar nichrome stimulating electrode was also placed in stratum radiatum for stimulation of Schaffer collateral afferents (0.05 ms duration). Unless otherwise mentioned, test stimuli were applied at a frequency of 1 per minute (0.017 Hz), and at a stimulus intensity that elicits a fEPSP slope that was 35% of the maximum. Experiments in which changes in the fiber volley occurred, were discarded. Drugs were applied through the perfusion medium. DL-AP5, calyculin A, KT5720 and R(+)-6-Bromo-7,8-dihydroxy-3-allyl-1-phenyl-2,3,5-tetrahydro-1H-3-benzazepine (6-Br-APB) were purchased from Research Biochemicals International, Natick, Mass.

### Example 2

Restricted and Regulated Overexpression Reveals Calcineurin as A Key Component in the Transition From Short-term to Long-term Memory

To investigate whether phosphatases play a role in memory storage, we assessed hippocampal-dependent memory in transgenic mice by expressing, primarily in the hippocampus, a truncated form of calcineurin. These mice have normal short-term memory but have a defect in long-term memory that is evident on both a spatial task (the spatial version of the Barnes maze) and on a visual recognition task, thus providing genetic evidence for the role of the rodent hippocampus in spatial as well as non-spatial memory storage. Further on the Barnes maze, the defect in long-term memory could be fully rescued by increasing the number of training trials. These results suggest that the transgenic mice overexpressing calcineurin have the capacity for long-term memory but have a specific defect in the transition between short- and long-term memory which prevents the storage of long-term memory. Using the tTA system, we next analyzed transgenic mice overexpressing calcineurin in a regulated manner and found that the memory defect observed is reversible and therefore is most likely due to the transgene and not to a developmental abnormality. Together with our electrophysiological findings that mice overexpressing calcineurin have a defect in an intermediate phase of long-term potentiation (I-LTP), our behavioral results suggest that calcineurin has a role in the transition from short- to long-term memory and that there is a correlation between this transition in memory storage and a novel intermediate phase of LTP.

Introduction

The insight that memory has time-dependent phases dates to 1890 when William James first proposed a distinction between a primary or short-term memory, a memory that has to be maintained continuously in consciousness, and secondary or long-term memory that can be dropped from consciousness and could be recalled at will at a later time (James, 1890). According to James view, short-term memory holds information for few seconds whereas long-term memory holds information for long periods of time. Subsequent experimental work suggested that these two phases of memory are usually in series and that the transition from short- to long-term memory is facilitated by an increase of the saliency or the number of training trials (Ebbinghaus, 1885; Weiskrantz, 1970; Craik and Lockhart, 1972; Wickelgren, 1973; Mandel et al., 1989).

The distinction between these two major phases was placed on a firmer biochemical basis when long-term memory was found to require the synthesis of new proteins, whereas short-term memory does not (Davis and Squire, 1984). These biochemical studies also revealed that short-term memory often lasted many minutes, and therefore was more enduring than the primary memory delineated by James. These studies therefore suggested that short-term memory may in turn have subdivisions, and that in addition to primary or working memory, there is a subsequent intermediate stage of, protein synthesis-independent, short-term memory. Further support for subcomponents of memory have also emerged from genetic studies in Drosophila and pharmacological studies in rodents and chicks (McGaugh, 1968; Cherkin, 1969; Gibbs and Ng, 1977; Frieder and Allweis, 1982; Rosenzweig et al., 1993; Tully et al., 1994; Zhao et al., 1995a and b; Bennett et al., 1996).

In addition to being able to distinguish temporal phases in memory storage, studies in human and monkey also delineated two distinct neural systems for long-term memory based upon the types of information stored. Bilateral lesions of the medial temporal lobe revealed an impairment in declarative long-term memory, a memory for people, places and objects but these lesions spared non-declarative memory for perceptual and motor skill. Particularly interesting was the finding that the lesions of the medial temporal lobe system, that interfere with declarative memory, only interfere with the long-term form of this memory and not with components of short-term memory, in particular not with working memory (Scoville and Milner, 1957; Mishkin, 1978; Zola-Morgan and Squire, 1985; Squire, 1987; Overman et al., 1990; Alvarez et al., 1994). These results indicate that structures in the medial temporal lobe, in particular the hippocampus, specifically subserve long-term memory but not some components of short-term memory.

In the preceding Example (Winder et al., 1998), we described mice that overexpress a truncated form of the phosphatase calcineurin in the hippocampus (lines CN98, Tet-CN279 and Tet-CN273). We found that these mice exhibit a specific defect in an intermediate phase of long-term potentiation (I-LTP). There is now increasing evidence that LTP can contribute to the storage of declarative forms of memory (Bliss and Collingridge, 1993; Eichenbaum, 1995, Mayford et al., 1996; Tsien et al, 1996). Like the temporal phases of memory, LTP also is not unitary but has at least two major phases: an early phase (E-LTP) elicited by a weak stimulus (1 train of 1s 100 Hz) and that is PKA- and protein synthesis-independent, and a late phase (L-LTP) induced by strong stimuli (4 trains of 1s 100 Hz) that requires PKA and protein synthesis (Huang and Kandel, 1994; Huang et al., 1996).

In addition to its role in the late phase of LTP, PKA is thought to be a component of a gate that regulates the initiation of LTP by opposing the actions of the phosphatases PP1 and PP2A (Blitzer et al, 1995; Thomas et al., 1996). Our electrophysiological results with mice expressing a truncated form of calcineurin are consistent with this idea and suggest that this gate has a distinct temporal component and forms a novel intermediate phase of LTP (I-LTP) that can be suppressed by calcineurin and that has three defining features: (1) it requires strong stimulation (a minimum of 2 train of ls 100 Hz) (2) it depends on PKA (3) it does not require protein synthesis.

In the present Example we assessed hippocampal-dependent memory in mice that express a truncated form of calcineurin. We find that mutant mice have normal short-term memory but exhibit a profound and specific defect in long-term memory on both the spatial version of the Barnes maze and on a task requiring the visual recognition of a novel object. To determine whether mutant mice have the capacity for long-term memory, we intensified the training protocol on the spatial version of the Barnes maze by increasing the number of daily training trials and found that the memory defect was fully reversed, indicating that these mice are capable of forming long-term memory. This rescue experiment suggests that mice overexpressing calcineurin have impaired long-term memory possibly due to a specific defect in the transition between short-term and long-term memory that may reflect a weakening of an intermediate component of memory.

Finally, we show that the memory defect observed was not the result of a developmental abnormality due to the genetic manipulation. In mice in which the expression of calcineurin transgene is regulated by the tetracycline-controlled transactivator (tTA) system, the spatial memory defect was reversed when the expression of the transgene was repressed by doxycycline.

Results

Mice Overexpressing Calcineurin Are Deficient on the Spatial Version of the Barnes Maze With one Trial a Day

In the previous Example (Winder et al., 1998) we described a physiological analysis of transgenic mice overexpressing calcineurin primarily in the hippocampus (line CN98) . This analysis revealed that CN98 mutant mice lacked an intermediate phase of LTP between the early, protein synthesis- and PKA-independent phase and the late, protein synthesis- and PKA-dependent phase. As a first step in analyzing the memory capability of these mice, we tested them on a hippocampal-dependent memory task: the spatial version of the Barnes maze (Barnes, 1979; Bach et al., 1995).

The Barnes maze is a circular maze that has 40 holes in the perimeter and a hidden escape tunnel placed under one of the holes. The mouse is placed in the center of the maze and is motivated to find the tunnel to escape the open brightly lit maze and an aversive buzzer. To locate the tunnel the mouse needs to remember and use the relationships among the distal cues in the environment. To achieve the learning criterion on this task the mouse must make three errors or less across five out of six consecutive trials. Errors were defined as searching any hole that did not have the tunnel beneath it. Previous research has established that performance on this task depends on the hippocampus (Barnes et al., 1979).

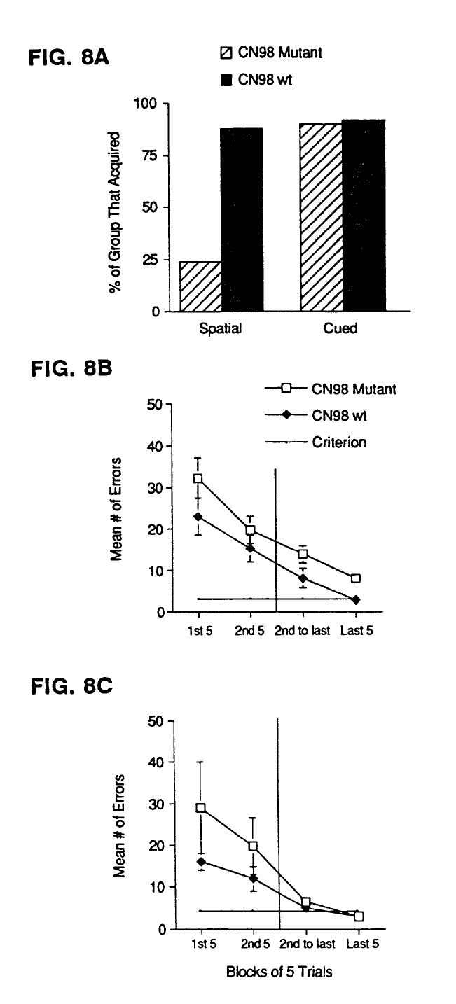

We tested CN98 mice on the Barnes maze once each day (1 trial per day, 24 h intertrial interval) until they met the learning criterion or until 40 consecutive days elapsed. Despite the fact that they were tested for 40 consecutive days, only 25% of the CN98 mutant mice met the learning criterion compared to 88% of the wild-type littermates (FIG. 8A). An analysis of the mean number of errors made across 4 blocks of 5 trials by mutant and wild-type mice revealed that the mutant mice made significantly more errors than wild-type mice across the last 2 trial blocks (Main effect genotype F[1,30]=4.63, p<0.05, FIG. 8B).

The impairment on the spatial version of the maze observed in the CN98 mutant mice could be due to a deficit in spatial memory or to a performance deficit such as a gross motor, visual or motivational impairment. To exclude a performance deficit, we next tested another group of CN98 mice on a cued version of the Barnes maze, a task which does not require the hippocampus. The cued version has similar contingencies and response requirements as the spatial version except that the position of the escape tunnel is made visible to the mice by putting a cue behind the hole where it is placed. Thus to locate the escape tunnel, the mice simply need to associate the cue with the tunnel. CN98 mutant mice acquired the task in a manner similar to that of their wild-type littermates (FIG. 8A) and made a similar number of errors across all trial blocks (Main effect genotype F[1,18]=2.44, p>0.05; FIG. 8C). These data indicate that CN98 mutant mice exhibit normal motivation and do not have any gross motor, motivational or visual impairments.

The Spatial Memory Deficit Can Be Fully Rescued Rescued by Repeated Training Trials

The results from the behavioral experiments on the spatial version of the Barnes maze which is a hippocampal-dependent task, indicate that CN98 mutant mice have a defect in spatial long-term memory. Have the mutant mice totally lost their ability to form long-term memory? Or do these mice have a block in the transition from short-term to long-term memory? Can the mice store long-term memory when trained with a more intensive protocol?

Our electrophysiological experiments indicated that L-LTP was reduced in CN98 mutant mice (Winder et al., 1998). Nevertheless, a potentiation similar to L-LTP could be induced by pharmacological agents that activate the PKA pathway. These results suggested that the machinery for the expression of L-LTP is intact in CN98 mutant mice and that the impairment seems to reside in an intermediate phase, between the early and the late phase, that is necessary for the production of the late phase (Winder et al., 1998). Since L-LTP is thought to parallel long-term memory (Abel et al., 1997), these results suggest that CN98 mutant mice may indeed have the ability to form long-term memory but may be deficient in an earlier phase of memory essential for the storage of long-term memory.

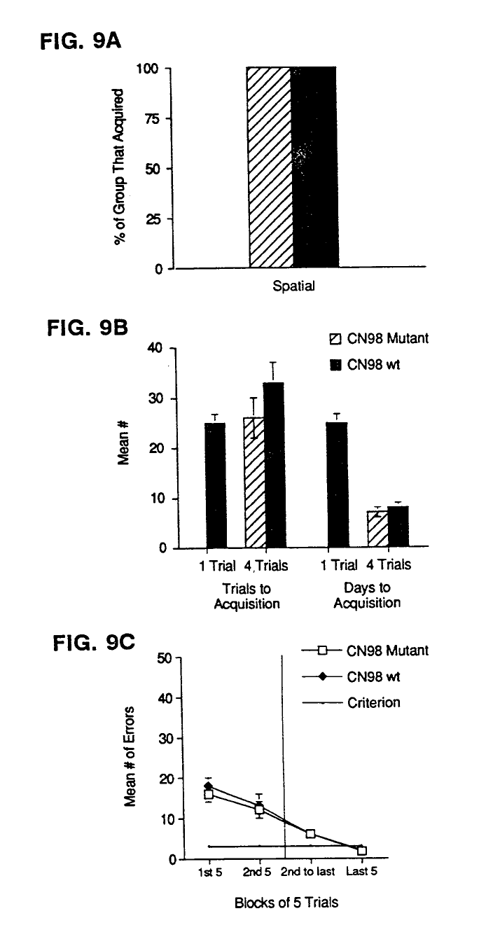

To test whether CN98 mutant mice have the capacity to fully acquire the spatial version of the Barnes maze, we modified the maze protocol by increasing the number of daily trials from one to four per day. The trials were separated by a 1.5 min intertrial interval. When trained with four trials per day, 100% of CN98 mutant mice were able to learn the spatial version of the Barnes maze as were 100% of wild-type mice (FIG. 9A). A comparison of the mean number of trials and days to criterion across the single versus repeated trials protocols revealed that a similar number of trials was required for the wild-type mice to learn the task whether a single or repeated trial was given each day (FIG. 9B) However, the number of days necessary for the acquisition of the task was much lower with four trials per day than with only one trial a day (FIG. 9B, results for mutant mice trained with one trial a day not shown since the majority did not acquire). An analysis of the mean number of errors revealed that mutant mice were similar to wild-type mice across all trial blocks (Main effect genotype F[1,8]=0.5191, p>0.05) (FIG. 9C).

These results demonstrate that CN98 mutant mice have impaired long-term memory on the spatial version of the Barnes maze when tested with one trial per day (24 h intertrial interval) but have normal long-term memory when tested with four trials per day (1.5 min intertrial interval) suggesting that CN98 mutant mice have the capacity for long term memory but have a deficiency in storing long-term memory. One possible interpretation of these results is that mutants have weak short-term memory that is taxed with one trial per day. By contrast, with four trials per day the short-term memory defect is overcome and normal retention occurs.

Short-Term Memory Is Normal in Mice Overexpressing Calcineurin

The demonstration that CN98 mutant mice have the capacity for hippocampal-dependent long-term memory when trained with repeated trials raised the question: Why do mutant mice have defective spatial memory when trained with one trial per day? Is short-term memory impaired? If so, can the defect in long-term memory be explained by a defect in short-term memory? Since spatial tasks such as the spatial version of the Barnes do not readily lend themselves to exploring short-term memory, we assessed the CN98 mutant mice for short term memory using a recognition task for novel objects. Spontaneous exploratory activity in rodents can be used as a measure of memory and in particular, it can be assessed to determine the recognition of a novel versus a familiar object in an object recognition task (Aggleton, 1985; Ennaceur and Delacour, 1988). In humans, the hippocampal region has been shown to play a role in the detection of novel visual stimuli (Tulving et al., 1996). Patients with hippocampal lesions exhibit impaired responses to novel stimuli (Knight et al., 1996; Reed and Squire, 1997). Results from studies on monkeys and rodents with hippocampal lesions suggest that the hippocampus may be important for novel object recognition (Myhrer, 1988a,b; Phillips et al., 1988; Mumby et al., 1995).

In the recognition task for novel objects, the mice were trained by being placed in a novel environment that contained two novel objects and were allowed to explore the objects for 15 min. During the testing phase, following different retention intervals, the mice were placed back in the environment but one of the two familiar objects was replaced with a third novel object. Mice with normal object recognition memory show an increase in exploration of the third novel object. This increase in exploration indicates that information regarding the familiar object was stored during training and further exploration of this object is no longer needed.

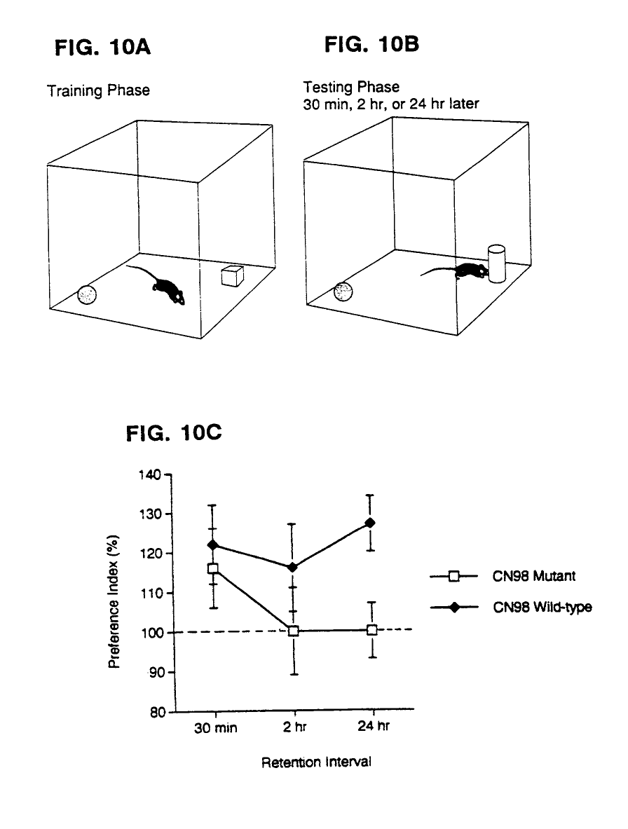

We first assessed exploration during the training phase by examining the amount of time spent exploring both novel objects and did not observe any difference between mutant and wild-type mice (Total initial exploration time, in seconds, for 30 minute run: wild-type 188 (10, mutant 142(26; 2 hour run: wild-type 148(16, mutant 141(10; 24 hour run wild-type 135(12 mutant 131(14; Main effect of genotype F[1, 67]=1.48, p=0.228). We then assessed exploration of the novel object following different retention intervals: short-term (30 min), intermediate-term (2 hr), and long-term (24 hr) . For this analysis, a preference index (PI) was determined by calculating the ratio between the amount of time spent exploring the novel object and the amount of time spent exploring both the novel and familiar objects during the first 5 min of the testing phase (the preference index was normalized and expressed as a percentage with PI=100% indicating no preference and PI greater than 100% indicating preference for the novel object). A significant difference in exploration of the novel object between mutant and wild-type mice was observed (Main effect genotype F[1, 67]=4.03, p=0.049). Post hoc analysis using a Student t test was performed for each retention interval and revealed that mutant mice exhibited an increase in exploration towards the novel object comparable to wild-type at 30 min (t=0.449, p>0.05) (FIG. 10). This indicates that the early components of short-term memory are intact in mutant mice. When mutant mice were tested at the 2 hr retention interval, they exhibited a slight memory defect compared to wild-type, although this difference was not significant (t=1.114, p>0.05) (FIG. 10). However, when tested at the 24 hr retention interval, mutant mice showed a long-term memory deficit that was statistically significant. Whereas wild-type mice exhibited a significant preference for the novel object, mutant mice explored both objects equally (t=2.061, p<0.05) (FIG. 10).

These results provide independent evidence for a deficit in long-term memory in CN98 mutant mice and suggest that the early components of short-term memory are intact. These results support the findings from the single versus repeated trial protocol in the Barnes maze in showing that mice overexpressing calcineurin have normal short-term memory and the capacity for long-term memory that is strengthened with repetition (four trials protocol) and allows long-term memory to be stored.

Calcineurin Overexpression Can Be Regulated by the tTA System

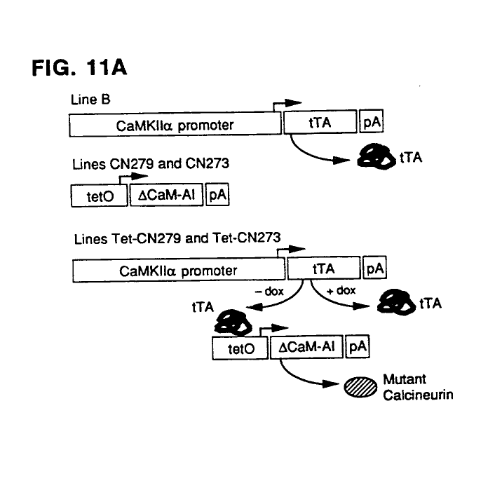

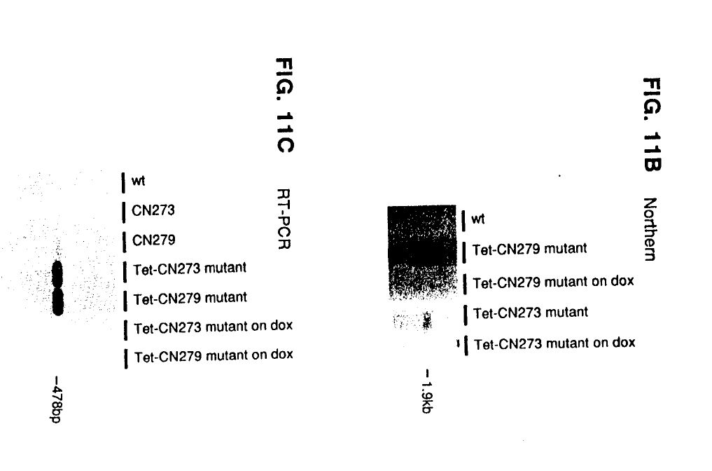

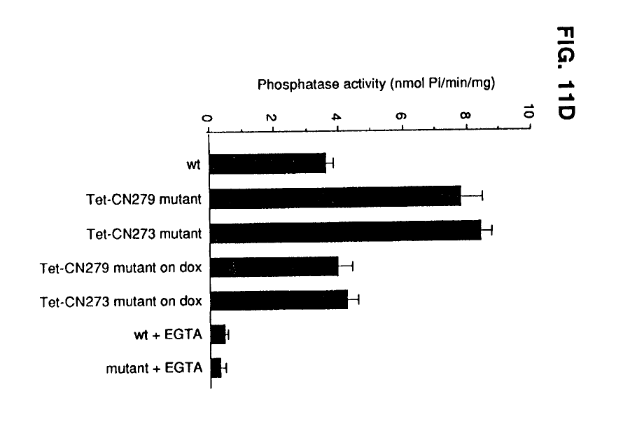

To verify that the memory impairment observed in CN98 mutant mice is not due to a developmental defect caused by the increase in calcineurin activity during postnatal development or to an effect of the insertion site of the transgene, we next assessed spatial memory in mice expressing the calcineurin transgene in a regulated manner under the control of the tTA system (lines Tet-CN279 and Tet-CN273 , FIG. 11A). To obtain regulated expression of the calcineurin transgene, we crossed mice that express the tTA gene under the control of the CaMKIIa promoter (line B, Mayford et al., 1996) with mice carrying the tTA-responsive promoter tetO fused to a cDNA encoding the truncated form of calcineurin ACaM-AI (lines CN279 and CN273 ) (FIG. 11A).

Northern blot analysis revealed a 1.9 kb transcript corresponding to the transgene mRNA in Tet-CN279 and Tet-CN273 mutant mice (FIG. 11B). By contrast, no signal was detected in mutant mice that received doxycycline in the drinking water (1 mg/ml in 5% sucrose) for at least one week or in wild-type controls (FIG. 11B). Further, a RT-PCR revealed expression of transgene mRNA in Tet-CN279 and Tet-CN273 mutant mice that was dramatically reduced when mutant mice were administered doxycycline for at least one week (FIG. 11B). Phosphatase assays revealed a 112%±9% and 114%±5% increase in Ca²⁺-dependent calcineurin activity respectively in Tet-CN279 and Tet-CN273 mutant compared to wild-type mice (FIG. 11C). This increase in phosphatase activity in Tet-CN279 and Tet-CN273 mutant mice was slightly higher than that detected in CN98 mutant mice (76%±12%, see Winder et al., 1998). In Tet-CN279 and Tet-CN273 mutant mice, phosphatase activity was suppressed to wild-type levels upon administration of doxycycline for at least one week (FIG. 11C).

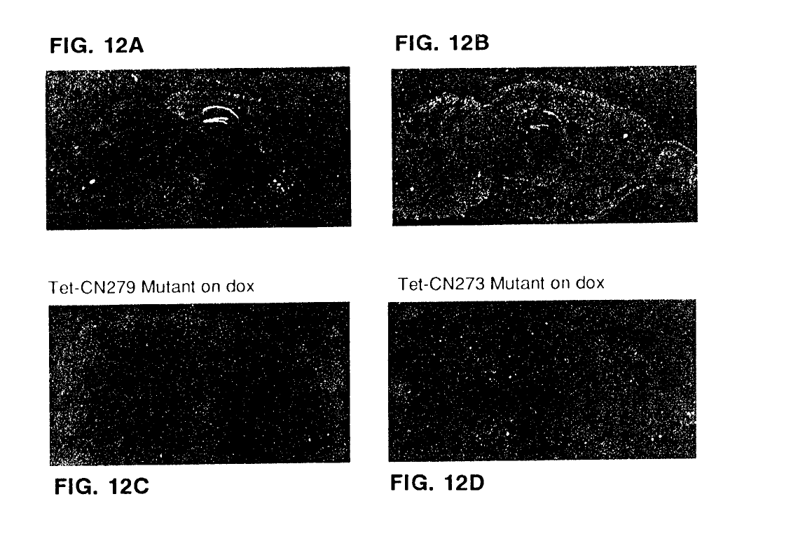

The spatial distribution of the transgene transcript was examined by in situ hybridization on adult brain in Tet-CN279 and Tet-CN273 mice. The transgene mRNA was detected mainly in the hippocampus and striatum, almost no expression was detected in neocortex. In the hippocampus, it was found primarily in area CA1 and dentate gyrus with relatively little expression in area CA3 (FIG. 12). In contrast, no signal was detected in mutant mice administered 1 mg/ml doxycycline for at least one week or in wild-type mice (FIG. 12).

The Memory Defect Can Be Reversed by Repression of the Calcineurin Transgene by Doxycycline

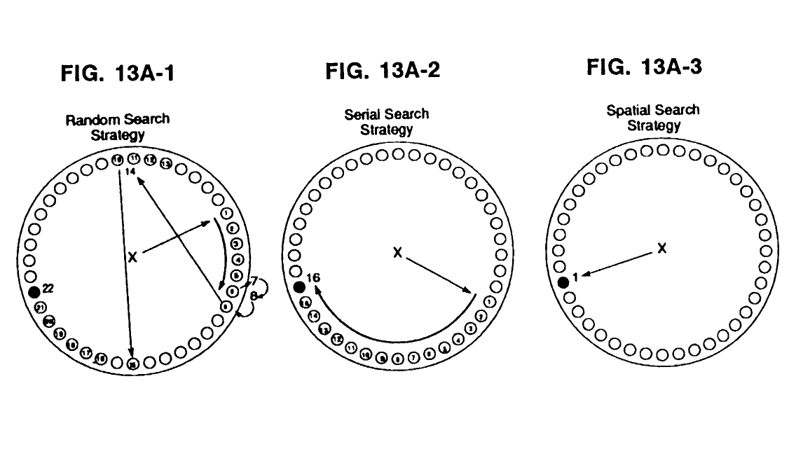

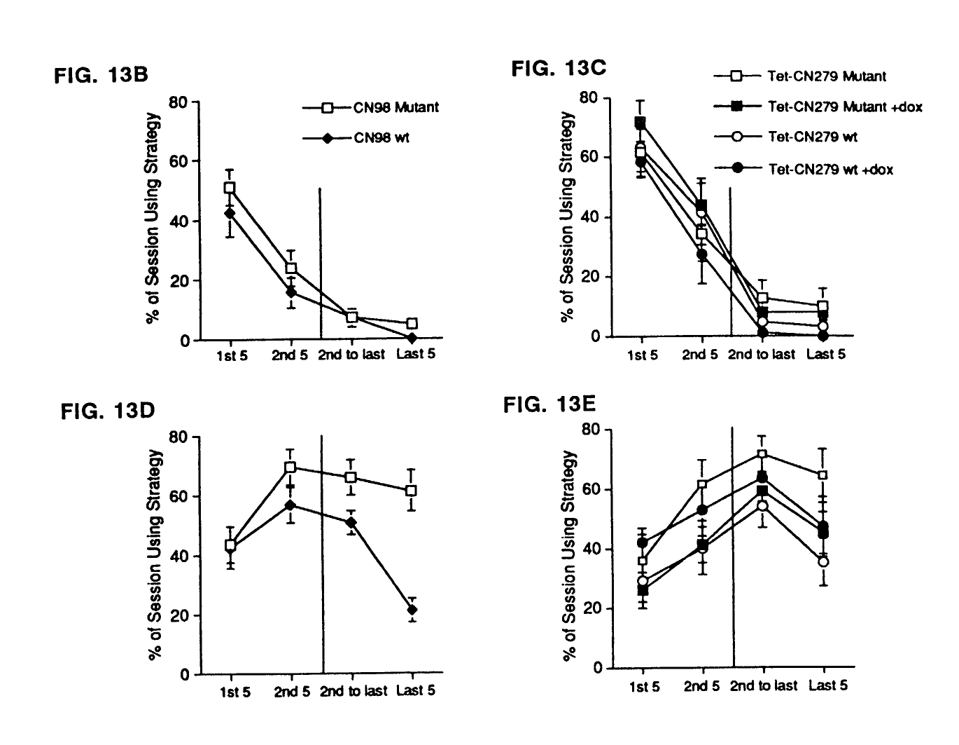

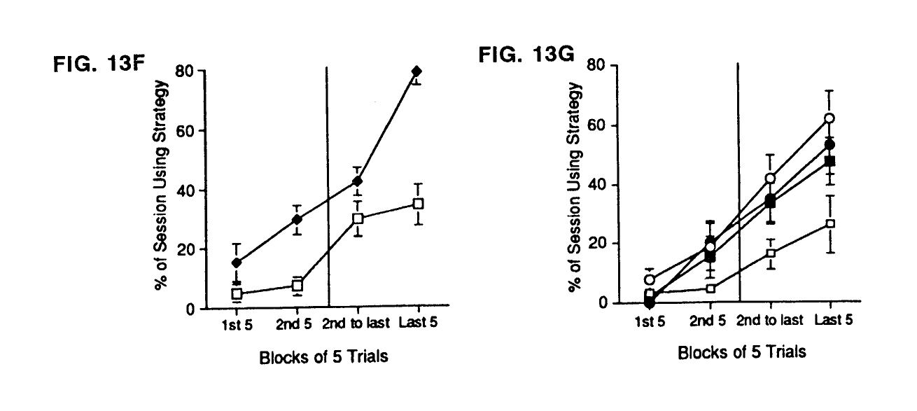

To assess whether the memory defect could be reversed by repression of calcineurin transgene with doxycycline in adult mice, we tested Tet-CN279 and Tet-CN273 mice on the spatial version of the Barnes maze. When performing the spatial version of the Barnes maze, mice normally progress through three search strategies: random, serial and spatial (Barnes, 1979; Bach et al., 1995) (FIG. 13A). The random search strategy is operationally defined as a random localized search of holes separated by center crossings which results in a large number of errors. The serial search strategy is defined operationally as a systematic search of consecutive holes in a clockwise or counter-clockwise fashion and use of the strategy results in less errors than for the random search strategy (FIG. 13A). The spatial search strategy, the most efficient strategy of the three and the only one that requires the hippocampus, is defined operationally as navigating directly to the tunnel with three of fewer errors (FIG. 13A). During the first 5 trials, CN98 and Tet-CN279 mutant mice (data not shown for the Tet-CN273 mice) and their respective wild-type mice either on or off doxycycline (FIGS. 13B and 13C) primarily used the random strategy and both exhibited a similar decrease in use across the remaining trial blocks (CN98: Main effect genotype by time F[3, 28]=0.5, p>0.05; Tet-CN279 : Main effect genotype F[1, 54]=1.63, p>0.05). The decrease in the use of the random strategy is paralleled by an increase in the use of the serial search strategy in CN98, Tet-CN279 mutant and wild-type mice. The serial strategy was employed significantly more often by CN98 and Tet-CN279 mutant mice during the last 2 trial blocks (FIGS. 13D and 13E) (CN98: Main effect genotype by time F[3, 28]=5.22, p<0.01; Tet-CN279: Main effect genotype by doxycycline F[1, 54]=6.12, p<0.05). By contrast, during the last 2 trial blocks, CN98 wild-type mice, Tet-CN279 mutant mice on doxycycline and wild-type mice employed primarily the spatial search strategy (FIGS. 13F and 13G) (CN98: Main effect genotype by time F[3, 28]=5.4, p<0.005; Tet-CN279: Main effect genotype F[1, 54]=4.64, p<0.05).

These results show that CN98 and Tet-CN279 mutant mice have a similar defect in spatial memory in that they do not employ the spatial search strategy. When the expression of the calcineurin transgene was repressed by doxycycline in Tet-CN279 mutant mice, this defect was reversed. The ability to reverse the memory loss suggests that the defect observed is probably not developmental but most likely due to expression of the calcineurin transgene and the resulting increase in calcineurin activity and its interference with memory storage in the adult brain.

Discussion

Calcineurin Plays a Role in Hippocampal-Dependent Memory: Transition from Short-term to Long-Term Memory

We found that mice expressing a truncated form of calcineurin exhibit a specific memory defect on the spatial version of the Barnes maze, a hippocampal-dependent task. No defect was observed on the cued version of the task, which is hippocampal-independent, indicating that the defect observed on the Barnes maze was in spatial memory and was not a motivational or sensory-motor defect. Further, the defect in spatial memory was reversible in adult mice overexpressing calcineurin in a regulated manner with the tTA system. These results provide the first genetic evidence that a phosphatase, and specifically calcineurin, has a role in hippocampus-based memory storage.

The data allow us to begin to delineate the components of memory that are affected and to identify components of memory that are not impaired. The results indicate that by increasing the number of daily trials on the spatial version of the Barnes maze, the long-term memory defect observed in the CN98 mutant mice was fully rescued. This shows that although they exhibit an apparent defect in spatial long-term memory, mutant mice indeed still have the capacity to store long-term memory. Although we cannot directly distinguish between a defect in long-term storage and a defect in the transition between short-term and long-term memory, the finding that the memory deficit observed with one trial a day can be rescued with repeated training suggest that mutant mice have a defect in some upstream processes required for the storage of long-term memory. These results therefore suggest that the short-term memory trace generated by a single daily trial disintegrates before the transition into long-term memory is complete. When the training is intensified so that the defective short-term trace is strengthened, long-term memory can be achieved.

Genetic Evidence Support the Notion that the Hippocampus Stores Some Aspects of Short-term as Well as Long-term Memory for Spatial and Non Spatial Tasks

Our results from the Barnes maze support those obtained on the novel object recognition task. On this task, the mutant mice have normal short-term memory at 30 min but have a significant defect in long-term memory at 24 hr. The combined results on the spatial version of the Barnes maze and the novel object recognition task further strengthen the hypothesis that the defect that leads to the impairment in long-term memory storage is a defect in the process or stages whereby short-term memory is converted into long-term memory. Since the calcineurin transgene is primarily expressed in the hippocampus, this defect in the transition very likely resides in the hippocampus. Whereas additional genetic manipulations would be required to establish this idea more firmly, the present results strengthen the important idea, well documented in humans and in primates (Scoville and Milner, 1957; Mishkin, 1978; Zola-Morgan and Squire, 1985; Overman et al., 1990), that the hippocampus is involved not only in the storage of long-term memory, but also in some aspects of the storing of short-term memory downstream from working memory. As a corollary, our experiments provide independent evidence that the rodent hippocampus is concerned with storing information other than space. In addition to showing a defect in spatial memory, genetic interference with I-LTP that is restricted to the hippocampus, also interfered with the recognition of novel object. These findings support the idea (Squire et al., 1992) that the rodent hippocampus is similar to that of humans in supporting a variety of memories that require the complex association of clues in all sensory modalities.

The Defect in the Transition From Short-term to Long-Term Memory Correlates With a Defect in I-LTP

Our behavioral and electrophysiological results suggest that an increase in calcineurin activity in the hippocampus leads to a defect in a transition phase of spatial memory between short-term and long-term memory as well as to a defect in a novel intermediate phase of LTP between early and late phase (Winder et al., 1998). Since short-term memory and E-LTP on one hand, long-term memory and L-LTP on the other have common properties in that short-term memory and E-LTP do not require protein synthesis whereas long-term memory and L-LTP depend on PKA and the synthesis of new proteins, our results showing a similarity in the behavioral and electrophysiological phenotypes suggest a correlation between the transition from short- to long-term memory and the novel intermediate phase of LTP. Our data also suggest a possible correlation between short-term memory and E-LTP since both are intact in our mice. Finally, our results extend further the correlation suggested between long-term memory storage and L-LTP (Abel et al., 1997). First, both long-term memory and L-LTP are impaired in our mice. Second, both long-term memory and L-LTP defects were rescued when the electrophysiological and behavioral protocols were systematically manipulated.

The Behavioral Rescue of Long-Term Memory Defect By Repeated Training Is Not Seen in CREB and CaMKII-Asp286 Mutant Mice

Repeated training experiments similar to those carried out here, have been performed in other genetically modified mice. In CREB knockout mice, the deficit in spatial long-term memory observed on the Morris water maze task was attenuated but not fully rescued by increasing the number of daily trials from 1 to 12 with 1 min intertrial interval, or from 1 to 2 with 10 min intertrial interval (Bourtchouladze et al., 1994; Kogan et al., 1996). However, when the interval between daily trials (2 trials per day) was increased to 60 min, performance in mutant mice was improved (Kogan et al., 1996). Further, mice overexpressing a constitutively active form of CaMKII (CaMKII-Asp286) were shown to have a spatial memory defect on the Barnes maze with one trial a day. In these mice, no improvement in spatial memory was observed when the number of trials was increased to 10 trials per day with 1 min intertrial interval and further, no improvement in performance was observed within a day across the 10 trials (Mayford et al., 1995). These results suggest that CREB knockout and CaMKII-Asp286 mutant mice may have spatial memory defects distinct from the defect observed in mice overexpressing calcineurin (a comparison of performance on the Barnes and Morris water maze is possible since both tasks involve similar cognitive processes). Specifically, CREB mutant mice have a defect in long-term memory although CaMKII-Asp286 mutant mice may have a defect in the formation of the short-term memory trace.

In turn, the behavioral deficits observed in mice overexpressing calcineurin and in CREB knockout mice provide an interesting comparison with mice expressing a dominant negative form of the regulatory subunit of PKA, R(AB) (Abel et al., 1997). In both mice overexpressing calcineurin and in R(AB) mutant mice, the PKA pathway is modified. In mice overexpressing calcineurin, the PKA pathway is affected indirectly through an increase in calcineurin activity which is suggested to suppress the PKA pathway (Winder et al., 1998) whereas in R(AB) mice, the PKA pathway is directly affected by the genetic manipulation since the activity of PKA itself is decreased. In CREB knockout mice, the defect appears to be further downstream from PKA since CREB has been implicated in the activation of gene transcription (Brindle and Montminy, 1992; Lee and Masson, 1993). Consistent with these three genetic manipulations acting on complementary sites, all three types of mice have a similar phenotype: short-term memory and E-LTP are normal but L-LTP and long-term memory are impaired.

Experimental Procedures

Barnes Circular Maze

Barnes maze experiments were performed as previously described with animals singly housed for at least three days before the first day of experiment (Bach et al., 1995). Thirty four CN98 mice (mutant: n=17, wild-type: n=17), 58 Tet-CN279 (mutant: n=14, on doxycycline n=20, wild-type: n=13, on doxycycline n=11) were tested on the spatial version of the Barnes maze. Thirteen CN98 mice (mutant: n=7, wild-type: n=6) were tested on the cued version of the maze. Briefly, the Barnes maze is a circular platform with forty holes at the periphery with an escape tunnel placed under one of the holes. On the first day of testing, each mouse was placed in the tunnel and left there for 1 min. The first session started 1 min after the training trial. At the beginning of each session, each mouse was put in a starting chamber in the center of the maze for 10 s and a buzzer was turned on. The start chamber was then lifted and the mouse was allowed to explore the maze. The session ended when the mouse entered the tunnel or after 5 min elapsed. The buzzer was then turned off and the mouse was allowed to stay in the tunnel for 1 min. In the spatial version of the maze, the tunnel was always located under the same hole which was randomly determined for each mouse. When tested with 4 trials per day, after being removed from the escape tunnel, the mouse was placed into the start chamber on the maze for 30 sec. Thus, each trial was separated by an intertrial interval of 90 sec (60 sec in the escape tunnel and 30 sec in the start chamber). In the cued version of the maze, the cue (an aerosol can) was placed directly behind the hole of the escape tunnel which was randomly determined for each mouse, each day. In both versions of the maze, the mice were tested once a day until they met the criterion of three errors or less on 5 out of 6 consecutive days or until 40 days elapsed. An error was defined as searching a hole that did not have the tunnel beneath it. The order of holes searched and the search strategy employed were manually recorded by an experimenter blind to genotype.

For both the spatial, cued and repeated trials versions, within the CN98 line, a two factor ANOVA (genotype and one repeated measure) was employed. For the Tet-CN279 line a three factor ANOVA (genotype, doxycycline and one repeated measure) was employed.

Novel Object Recognition Task

Seventy-three mice from the CN98 line (mutant: 30 min n=9; 2 hr n=12; 24 hr n=15; wild-type: 30 min n=9; 2 hr n=11; 24 hr n=17) were individually assessed on the novel object recognition task. Three mutant and three wild-type mice were excluded because they displayed a strong preference (Preference index<60) towards the familiar object during both training and testing. During the training trial, mice were placed in a square novel environment (20″ long by 8″ high) constructed from plywood and painted white with epoxy paint. Two (of three possible) plastic toys (between 2.5 and 3 inches) that varied in color, shape and texture were placed in specific locations in the environment 14 inches away from each other. Two different combinations of object pairs were counterbalanced across genotype and retention intervals. The mice were able to freely explore the environment and objects for 15 min and then were placed back into their individual home cages. Following various retention intervals (30 min, 2 hr or 24 hr) , mice were placed back into the environment with two objects in the same locations but now one of the familiar objects was replaced with a third novel object. The mice were then again allowed to freely explore both objects for 15 min. The objects were thoroughly cleaned with a mild detergent (Roccal diluted 1:50 in water) before each experiment to avoid instinctive odor avoidance due to mouse's odor from the familiar object. During both training and testing phases, an experimenter blind to genotype recorded the number of seconds spent exploring each individual object for each minute across 15 min. A mouse was considered exploring the object when its head was facing the object at a distance of 1 inch or less or when any part of its body except the tail was touching the object. For the purpose of data analysis we added the total number of seconds spent exploring each object for the first 5 min during the testing phase and calculated a preference index (PI). The amount of time spent exploring the novel object was divided by the amount of time exploring both the novel and familiar objects. The resulting value was divided by 0.5 which represents no preference for either object and that result was then multiplied by 100. A PI greater than 100 indicates preference for the novel object during testing. A PI equal to 100 indicates no preference whereas a PI inferior to 100 indicates a preference for the familiar object. A two factor ANOVA (genotype and one repeated measure) and individual Student t tests for each retention interval were employed to assess the effect of genotype on the PI at the different retention intervals.

Plasmid Construction

Construction of the plasmid used to generate the CN98 mice is described in Winder et al., 1998. For the generation of Tet-CN279 and Tet-CN273 mice, a plasmid was constructed with a cDNA encoding a truncated and active form of the murine calcineurin catalytic subunit Aα, ΔCaM-AI. ΔCaM-AI lacks the autoinhibitory domain and a portion of the calmodulin-binding domain of calcineurin Aα and was shown to be constitutively active in Jurkat T-cells (O'Keefe et al., 1992). A 1.27 kb EcoRI fragment of ΔCaM-AI cDNA was made blunt-ended and subcloned into the EcoRV site of pNN265 vector. The plasmid pNN265 carries upstream from the EcoRV site, a 230 bp hybrid intron that contains an adenovirus splice donor and an immunoglobulin G splice acceptor (Choi et al., 1991) and has a SV40 polyadenylation signal downstream from the EcoRV site. The ΔCaM-AI cDNA flanked by the hybrid intron in 5′ and the poly(A) signal in 3′ was excised from pNN265 with NotI and the resulting 2.7 kb fragment was placed downstream of teto promoter from plasmid pUHD10-3 (Gossen and Bujard, 1992) to generate CN279 and CN273 mice (FIG. 11A) . The final 3.1 kb tetO-ΔCaM-AI (FIG. 11A) fragment was excised from the vector by NotI digestion. Prior to microinjection, all cloning junctions were checked by DNA sequencing.

Generation and Maintenance of Tet-CN279 and Tet-CN273 Transgenic Mice

The transgenic mice CN279 and CN273 were generated by microinjection of the linear construct as previously described (Hogan et al., 1994; Winder et al., 1998). Analysis of founder mice for integration of the transgene was performed by Southern blotting and PCR. The founder mice were backcrossed to C57BL6 F1/J mice to generate the transgenic lines CN279 and CN273. To generate Tet-CN279 and Tet-CN273 mice, CN279 and CN273 F1 mice were crossed with CaMKIIα promoter-tTA mice (line B, Mayford et al., 1996) (FIG. 11A). The offspring was checked by Southern blotting or PCR. Transgenic mice were maintained in the animal colony according to standard protocol. Tet-CN279 and Tet-CN273 mice were administered either water or 1 mg/ml doxycycline (in 5% sucrose) in the drinking water at least one week before being used.

Northern Blot

Northern blot analysis was performed as described in Winder et al., 1998. Briefly, forebrains from adult Tet-CN279 and Tet-CN273 mice administered water or doxycycline were collected and total RNA was isolated by the guanidinium thiocyanate method (Chomczynski and Sacchi, 1987). Ten micrograms of RNA were denatured, electrophoresed on a 1% agarose gel and transferred to a nylon membrane in 0.4 N NaOH. The membrane was hybridized overnight at 42_C. to a radiolabeled 1.1 kb EcoRV-NotI fragment from pNN265, washed and exposed to film for three days.

RT-PCR

For RT-PCR, total RNA from forebrain was amplified according to the manufacturer's protocol (GIBCO BRL®). Briefly, cDNA was synthesized from 3 μg of total RNA with the Superscript II RT in a 20 μl reaction. Amplification was performed with Taq Polymerase (BOEHRINGER MANNHEIM®) for 25 cycles as follows : 94° C. for 30s, 50° C. for 30s and 72° C. for 1 min. The following oligonucleotides were used as primers: 5′-CCTGCAGCACAATAATTTGTTATC-3′ (Seq I.D. No. 2) and 5′-TAGGTGACACTATAGAATAGGGCC-3′ (Seq I.D. No. 3). They produced a 478 bp fragment containing 406 bp of ΔCaM-AI cDNA and 72 bp of pNN265 sequences. Samples were run on a 2% agarose gel then transferred onto NYLON® membrane. The membrane was hybridized to [α³²P]dCTP-labeled probe specific for pNN265 sequences in the PCR product. Hybridization was performed overnight at 42° C. in 50% formamide, 2× SSC, 1% SDS, 10% dextran sulfate, 0.5 mg/ml denatured salmon sperm DNA. The membrane was washed 10 min at room temperature in 2× SSC, 1% SDS, twice 15 min at 42° C. in 2× X SSC, 1% SDS then twice 15 min at 42° C. in 2× SSC, 1% SDS, 0.2× SSC and exposed to film.

In Situ Hybridization

In situ hybridization were performed as described in Winder et al., 1998. Briefly, brains from adult Tet-CN279 and Tet-CN273 mice either on or off doxycycline were dissected out and sectioned. Sections were fixed 10 min in 4% paraformaldehyde, rinsed in PBS and dehydrated. Sections were rehydrated, permeabilized, washed and rinsed before being hybridized overnight at 37° C. to a [α³⁵S] ATP-labeled oligonucleotide (5′-GCAGGATCCGCTTGGGCTGCAGTTGGACCT-3′) (Seq I.D. No. 4) specific for the transgenes. After hybridization, slides were washed, dehydrated then exposed to Kodak Biomax MR film for 2 to 3 weeks.

Phosphatase Assay

Phosphatase assays were performed as described in Winder et al., 1998. Briefly, mice were injected with 5 ml/kg of pentobarbital and decapitated. Hippocampi were dissected out, homogenized in 2 mM EDTA (pH 8), 250 mM sucrose, 0.1% β-mercaptoethanol. Supernatants were incubated at 30° C. for 1 min in presence of the [α³²p] -labeled [Ala97]-RII peptide and either 0.1 mM calmodulin and 0.66 mM Ca²⁺ or 0.33 mM EGTA. The reaction was stopped and the enzyme activity calculated previously as described (Klee et al., 1983; 1987). The activity was expressed in nmol Pi released/min/mg protein. The protein concentration was determined using the bicinchroninic acid protein assay kit (SIGMA®). All samples were performed in triplicate.

### Example 3

Inducible and Reversible Gene Expression with the rtTA system for the Study of Memory

To obtain rapidly inducible and reversible expression of transgene in forebrain of the mouse, we have combined the reverse tetracycline-controlled transactivator (rtTA) system with the CaMKIIα promoter. Using calcineurin and a reporter gene, we show that doxycycline induces maximal expression of the transgene within six days. Expression of calcineurin in turn leads to an impairment in an intermediate form of LTP (I-LTP) in hippocampus and to a defect in spatial memory in the Morris water maze. Mutant mice that express the calcineurin transgene transiently, after spatial memory was stored, have an apparent defect in the retrieval of the spatial information. This retrieval defect is not due to a disruption in memory storage since it could be reversed when the transgene expression was turned off by doxycycline removal. These results demonstrate that the rtTA system can be used as a reversible genetic switch to examine time-dependent memory processes.

The ability to regulate the expression of transgenes in the brain of genetically modified mice has significantly advanced the study of gene function on both an electrophysiological and behavioral level. The tetracycline-controlled transactivator (tTA) system, based on a transcriptional activator tTA and a tTA-responsive promoter, tetO, provides a system by which the expression of a transgene can be suppressed by tetracycline or its analogs (Gossen and Bujard, 1992). By combining the tTA system with the forebrain-specific CaMKIIα promoter, Mayford et al. were able to achieve regulated transgene expression in restricted areas of the brain (Mayford et al., 1996a and b; see also Mansuy et al., 1998). However, the tTA system suffers from the disadvantage that in the absence of doxycycline, the transgene is expressed. Thus, to prevent transgene expression during development, doxycycline would have to be administered to the mother throughout gestation and this chronic administration of doxycycline interferes with normal memory (Mayford et al., 1996b).

Recently, Gossen et al. (1995) developed a novel transactivator, the reverse tTA (rtTA) by random mutagenesis of tTA. In the presence of tetracycline analogs, rtTA is able to activate the transcription of a gene placed downstream from tetO. With the non-specific human cytomegalovirus immediate early gene (CMV) promoter, rtTA rapidly allowed to induce expression of a reporter gene in various organs of adult mice by administration of doxycycline (Kistner et al., 1996).

We now have succeeded in applying the rtTA system to the brain by combining it with the CaMKIIα promoter and have used it to reversibly induce gene expression. We found that the expression of a lacZ reporter gene and of a transgene coding for a truncated and active form of the Ca²⁺-dependent phosphatase calcineurin (PP2B), ΔCaM-AI, is rapidly induced in hippocampus, cortex and striatum by administration of doxycycline in the food in two independent lines of mice (O'Keefe et al., 1992). The induction of calcineurin overexpression in brain led to a specific defect in an intermediate form of long-term potentiation (I-LTP) in area CA1 of the hippocampus and interfered with memory storage (Mansuy et al., 1998; Winder et al., 1988). By temporally manipulating the calcineurin transgene expression, we also provide evidence that an excess of calcineurin interferes not only with the storage but also with the retrieval of spatial memory.

Results

Doxycycline Leads to the Induction of rtTA-Driven Transgene Expression

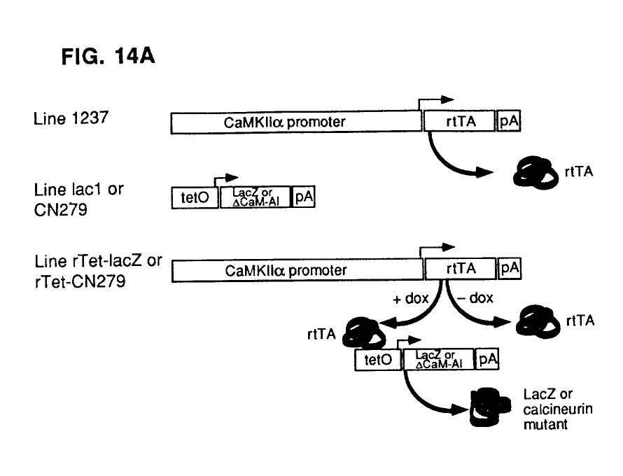

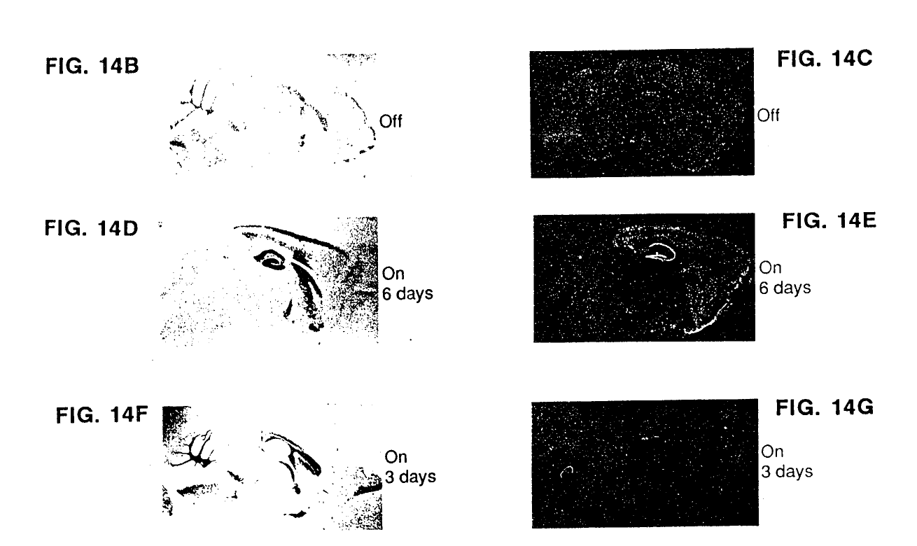

To adapt the rtTA system to brain, we first generated mice expressing rtTA under the control of the CaMKIIα promoter (lines 1237 and 1076, no results are shown for line 1076 since they were similar to 1237). To examine the pattern and the time course of induction of transgene expression with the rtTA system, we used the lacZ reporter gene and crossed the mice expressing rtTA in forebrain (from line 1237) with mice carrying a tetO promoter-lacZ reporter construct (line lacd) (FIG. 14A, Mayford et al., 1996b).

In mice carrying both CaMKIIα promoter-rtTA and tetO-lacZ transgenes (rTet-LacZ from crossing between line 1237 and line lac1), we assessed the induction of the lacZ reporter gene expression in vivo. We found that to obtain full induction of the expression of the reporter gene in adult mouse brain, six days of treatment with doxycycline in the diet (6 mg/g of food) were necessary. After a 6-day treatment, lacZ expression was induced in CA1 and CA2 areas with almost no signal in CA3 region of hippocampus, in dentate gyrus, in superficial layers and in a deep layer of cortex, in septum and striatum (see FIG. 14B (on) for line 1237). No staining was detected in untreated mice carrying both transgenes (see FIG. 14B, off) suggesting there was little or no activation of the transgene expression by rtTA in the absence of doxycycline.

Whereas the same pattern of lacZ gene expression was obtained after a 6-day treatment with a higher dose of dox (12 mg dox/g of food), only a modest induction of expression, primarily in striatum, was observed after 6 days of treatment with 3 mg/g of food or after 3 days of treatment with 6 mg/g of food.

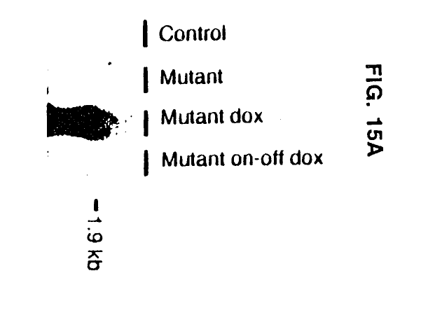

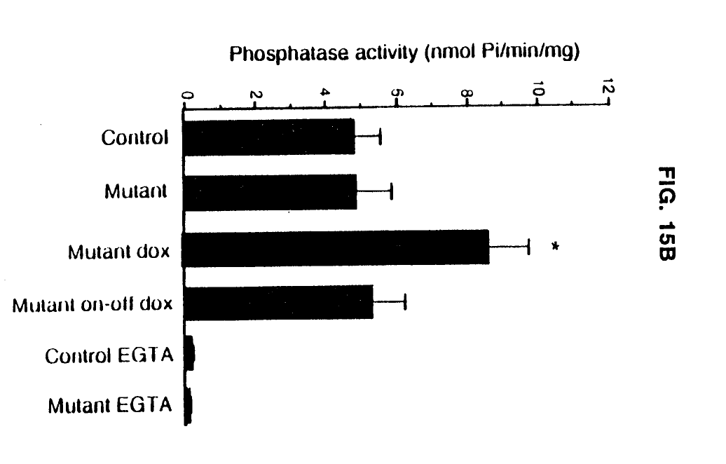

In addition to a reporter gene, we also generated mice expressing a calcineurin transgene, ΔCaM-AI (line CN279, Mansuy et al., 1998) under the control of the rtTA system (FIG. 14A) . Overexpression of this form of calcineurin leads to a defect in an intermediate form of LTP that is dependent on cAMP-dependent protein kinase A (PKA) in hippocampus (Winder et al., 1998) and to an impairment in both spatial and non-spatial hippocampal-based memory (Mansuy et al., 1998). Mutant mice carrying both CaMKIIα promoter-rtTA (line 1237) and tetO-DCaM-AI (line CN279 ) transgenes (rTet-CN279 ) were treated with doxycycline (6 mg/g of food) for at least 6 days. In situ hybridization revealed that doxycycline induced expression of the calcineurin transgene in a pattern somewhat broader than evident with the reporter gene. The calcineurin transgene was expressed in areas CA1, CA2 and CA3 of the hippocampus, in all cortical layers except layer IV, and in striatum (FIG. 14C, on). No signal was detected in the brain of mutant mice not treated with doxycycline (FIG. 14C, off). In mutant mice treated with doxycycline for 2 weeks, the transgene mRNA could be detected (FIG. 15A) and was accompanied by a 77%±10.7% increase in calcineurin activity (FIG. 15B). Northern blot analysis and in situ hybridization showed that the transgene expression was stable as long as doxycycline was maintained in the diet. To determine whether the expression of the calcineurin transgene could be reversed by removing doxycycline from the diet, Northern blot analysis and phosphatase activity assays were performed on hippocampal extracts from mice withdrawn from doxycycline for 2 weeks after a 2-week treatment (6 mg/g of food) (FIGS. 15A-15B). Two to three weeks after doxycycline was removed from the diet, no transgene mRNA was detected (FIG. 15A) and the calcineurin activity was reduced to basal levels (FIG. 15B). In untreated mutant mice no transgene mRNA or enhanced phosphatase activity was detected, which indicates that in this line of mice, there was no transactivation by rtTA in the absence of doxycycline (FIGS. 15A-15B).

Induction of Calcineurin Transgene Expression Leads to a Defect in I-LTP in Schaffer Collateral Pathway

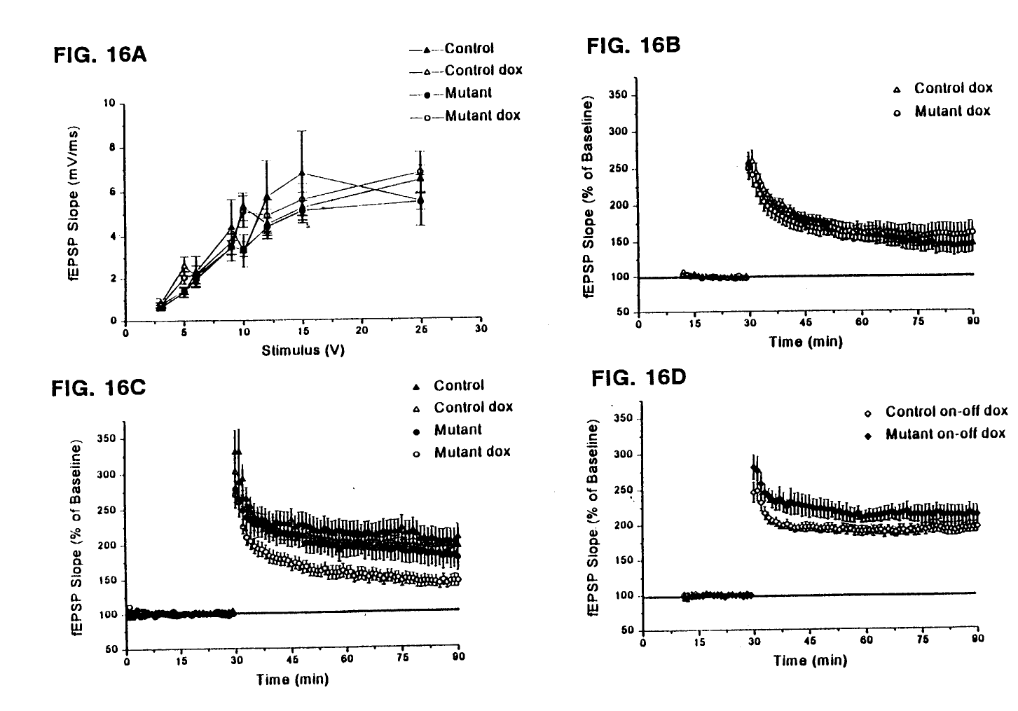

We have previously demonstrated that overexpression of calcineurin either constitutively or under the control of the tTA system results in a specific defect in an intermediate phase of LTP (I-LTP) induced by two 100_Hz trains at the Schaffer collateral CA1 pathway with no defect in the PKA-independent form of LTP (E-LTP) (se above examples). We examined the consequence of overexpression of the calcineurin transgene under the control of the rtTA system in adult hippocampus by measuring basal synaptic transmission and synaptic plasticity. Although basal synaptic strength as measured by comparing input-output curves of Schaffer collateral stimulation, and LTP induced by one 100 Hz train (E-LTP) were not perturbed by the expression of the calcineurin transgene (FIGS. 16A and 16B), LTP induced by two 100 Hz trains (I-LTP) was impaired (% of baseline at 1 hour: Control, 208±18; Control dox, 195±9; Mutant, 184±17; Mutant dox, 141±8, Mutant dox versus Mutant, p<0.05, Mutant dox versus Control [dox or no dox], p<0.001, FIG. 16C). The observed defect was the direct consequence of the transgene expression and was not due to doxycycline itself since LTP induced by two 100 Hz trains was normal in doxycycline-treated hippocampal slices from control mice. Moreover, the defect in LTP induced by two 100 Hz trains was reversed when the expression of the calcineurin transgene was turned off by removal of doxycycline for two weeks (FIG. 16D).

Induction of the Calcineurin Transgene Expression in Forebrain During Training Impairs Spatial Memory

We had earlier shown that the constitutive overexpression of calcineurin in transgenic mice interferes with spatial memory in the Barnes maze (Mansuy et al., 1998). In these mice, short-term memory was normal but the transition between short-term and long-term memory was affected. We now have extended this analysis to another similar spatial task, the Morris water maze (Morris, 1982) and examined the consequence of the overexpression of calcineurin induced by doxycycline in rTet-CN279 mice. The Morris water maze is a hippocampal-dependent behavioral task that requires mice to learn and remember the relationship between distal cues in the environment to locate a hidden escape platform submerged in a pool filled with opaque water (Morris, 1982).

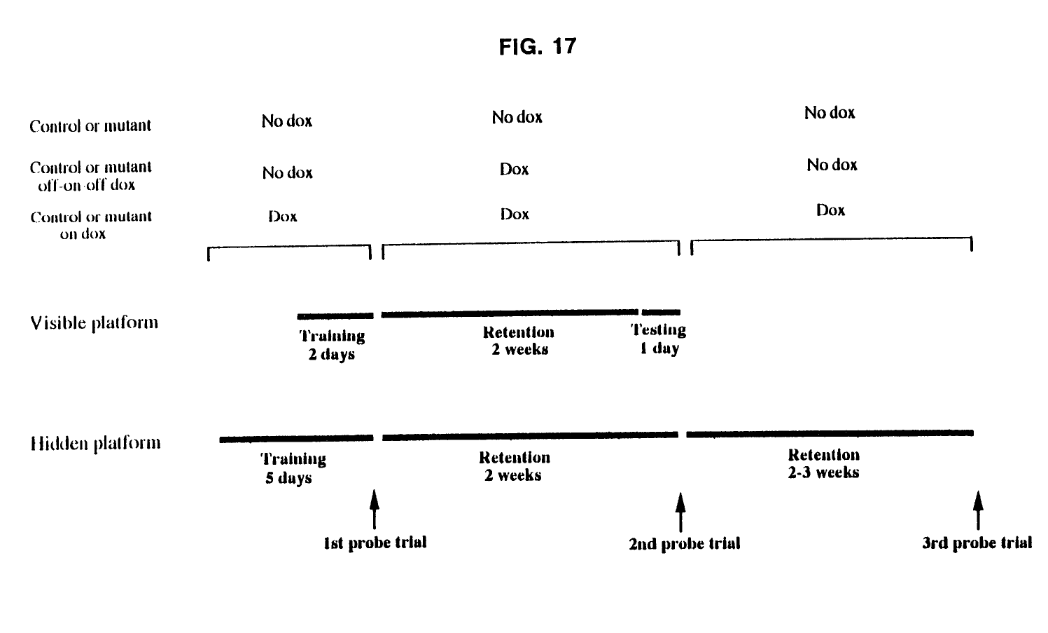

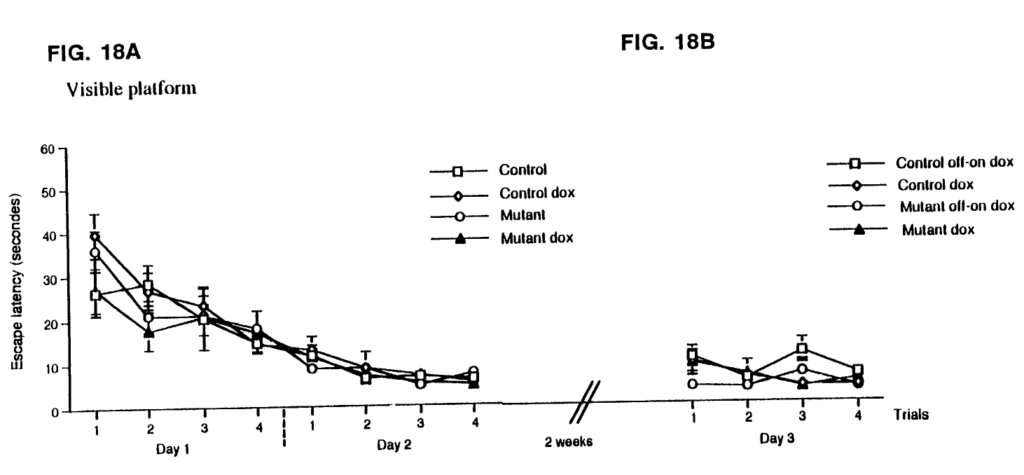

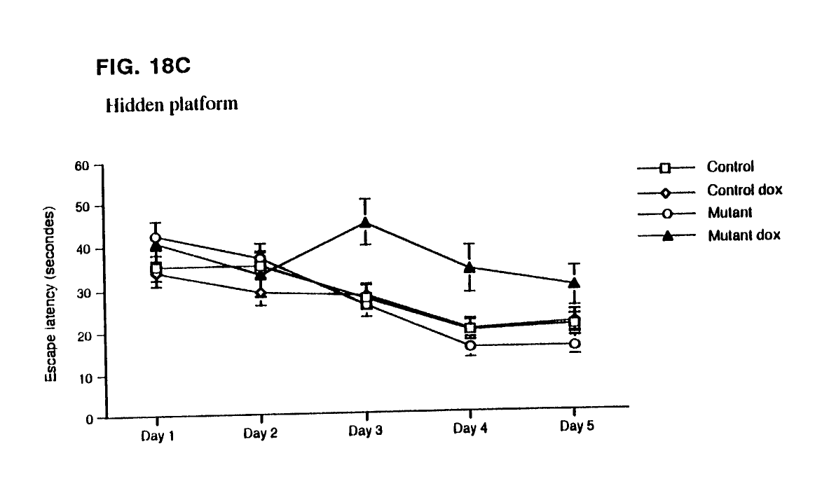

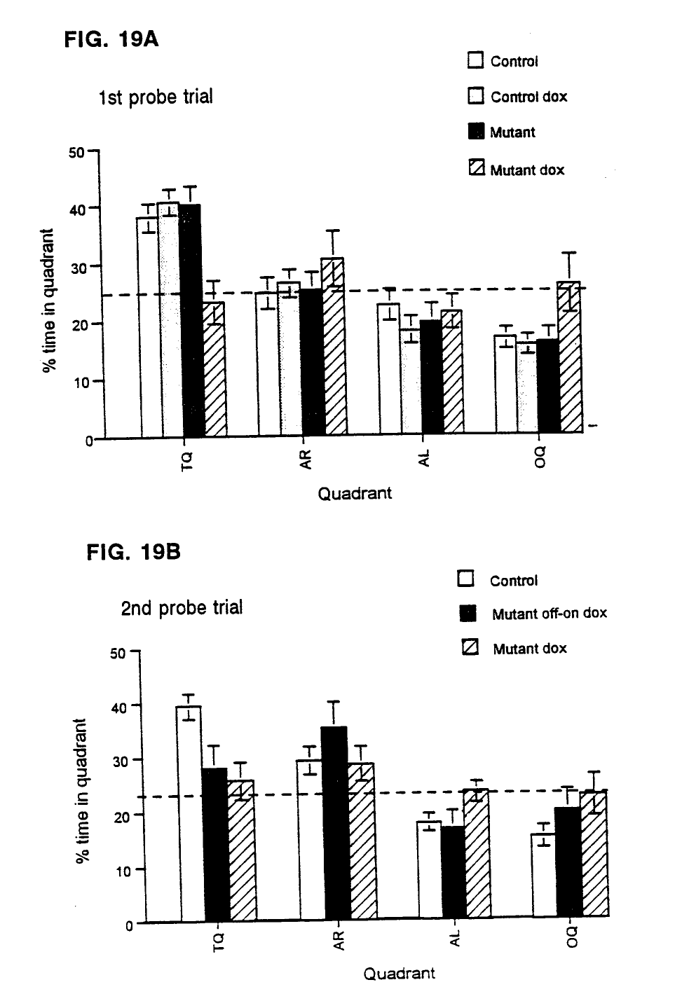

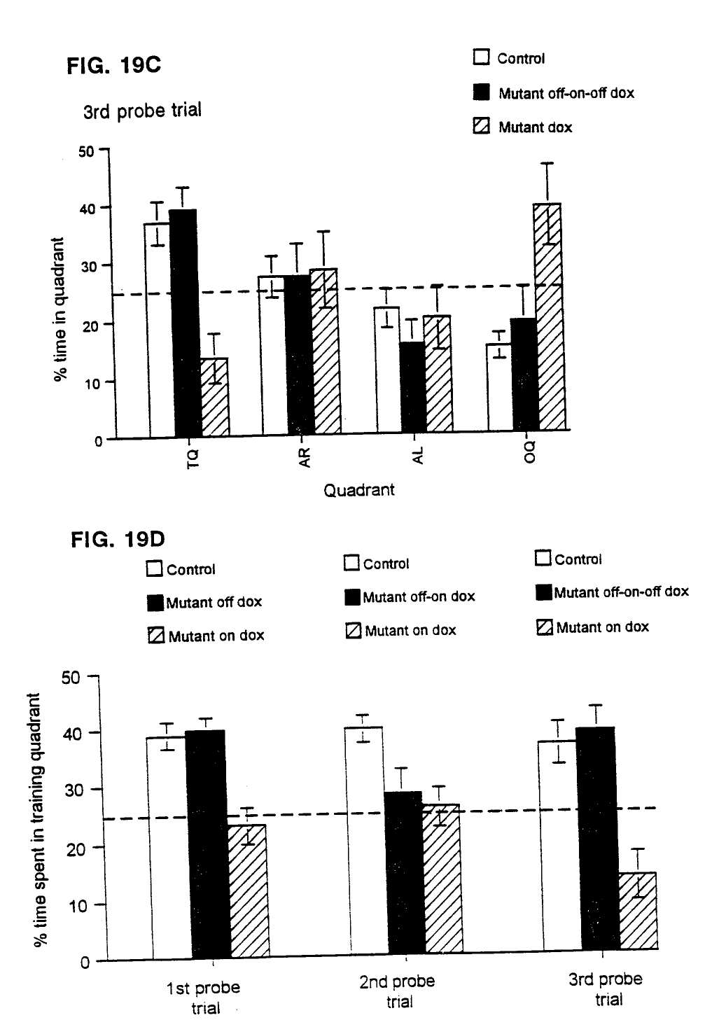

The rTet-CN279 mice were initially tested on a hippocampal-independent cued version of the Morris maze in which they learn to associate the platform with a proximal and visible cue placed onto the platform (see diagram FIG. 17). On this task, learning is assessed by measuring the time spent swimming to reach the visible platform (escape latency). On the visible platform version of the Morris water maze, escape latencies decreased across the 2-day training (4 trials per day) for both control and mutant mice. No difference was observed between control and mutant mice independent of whether or not they received doxycycline indicating that doxycycline and transgene expression did not interfere with learning or performance (FIG. 18A).

We next tested mice for hippocampal-based spatial memory using the hidden platform version of the maze. Mice were trained for 5 days with 4 trials per day (see diagram, FIG. 17). Across the 5-day training, both control mice treated or not treated with doxycycline and mutant mice not treated with doxycycline, thus not expressing the transgene, showed a similar gradual decrease in escape latency (FIG. 18C). By contrast, the mutant mice treated with doxycycline showed no improvement in performance and their escape latencies remained high across training. After the 5-day training, memory for the position of the platform was assessed on a first probe trial (see diagram FIG. 17) where the platform was removed from the pool and the search time of mice allowed to swim for 60 sec was recorded in each quadrant of the pool. Control mice whether treated or not treated with doxycycline, and mutant mice not treated with doxycycline spent most of their time searching for the platform in the quadrant where it was placed during training (training quadrant). By contrast, mutant mice expressing the transgene did not spend more time searching in the training quadrant than in the other quadrants (FIG. 6A). Mutant mice expressing the transgene also exhibited a significant reduction in the number of times they swam across the site where the platform was placed during training (platform crossings, FIG. 19B). No difference in performance was observed between control mice treated or not treated with doxycycline and untreated mutant mice (FIGS. 19A and 19B) . These data demonstrate that a relatively transient overexpression of calcineurin is sufficient to produce deficits in hippocampal-dependent learning and memory.

Induction of the Calcineurin Transgene Expression After Training Reversibly Impairs Retrieval of Spatial Memory

The power to turn a transgene on and off allows one to probe the various components of the memory processes. In particular, it allows one to begin to examine specific aspects of learning and memory such as retrieval. We thus examined whether expression of the calcineurin transgene can perturb the retrieval of spatial memory.

We trained the rTet-CN279 mice on both the visible and the hidden platform version of the Morris water maze task in the absence of doxycycline and assessed their memory after training was completed. We then induced the expression of the calcineurin transgene with doxycycline immediately after training for 2 weeks and re-assessed their memory for the position of the platform on both tasks two weeks later (see diagram FIG. 17). On the visible platform version of the maze, we observed that both control and mutant mice, whether treated or not treated with doxycycline, had short escape latencies across the 4 trials on testing day that were similar to latencies observed at the end of training. Thus in the mutant mice, expression of the calcineurin transgene across the 2-week retention did not impair the retrieval of information about the visible platform learned during training. Furthermore, we observed no difference in performance between control and mutant mice whether treated with doxycycline during training or only after training (FIG. 18B).

In contrast on the hidden platform version of the task, mutant mice that performed well on the first probe trial and that expressed the transgene only after the first probe trial and across the 2-week retention, then failed to remember the position of the platform when tested on a second probe trial two weeks later. The mice spent less time searching for the platform in the training quadrant (FIG. 19C) and showed a trend to cross the site where the platform was located less often than control mice (FIG. 19D).

These results per se did not indicate, however, whether the defect observed on the second probe trial reflected a disruption of the previously established memory storage or consolidation or whether it reflected a defect in the retrieval of the stored information. To address this question, mutant mice that performed well on the first probe trial but poorly on the second one after the expression of the calcineurin transgene was induced, were tested on a third probe trial 2 to 3 weeks after the second one and after the transgene expression was turned off again by removal of doxycycline (see diagram FIG. 17). On the third probe trial, these mice were able to remember the position of the platform they had learned during training and spent approximately the same time in the training quadrant as control mice treated with doxycycline during training and retention or treated only between the first and second probe trial (FIG. 19E). They also crossed the site where the platform was originally placed a similar number of times as control mice (FIG. 19F). In both control mice treated or not treated with doxycycline and in mutant mice not treated with doxycycline, an overall decrease in performance on the third probe trial was also observed when compared to the second probe trial.

These results (see FIG. 19D for summary of performance on training quadrant) indicate that mutant mice that did not express the transgene had normal storage of long-term spatial memory and that the induction of the calcineurin transgene expression interfered with the retrieval of normally stored spatial memory but had no effect on the retrieval of non-spatial memory.

Discussion

The rtTA System Allows Rapid and Reversible Expression of Transgenes

The tTA-regulated expression system has previously been used to confirm that calcineurin and CaMKII play a role in synaptic plasticity and in memory storage (Mayford et al., 1996b; Mansuy et al., 1998; Winder et al., 1998). However, the tTA system suffers from two disadvantages which have limited its use. First, the transgene is always activated unless doxycycline is administered. Second, once administered, doxycycline is stored in both muscle and bone and therefore, is not easily washed out of the body. As a result, it can take a long time to reactivate the expression of the transgene after it has been suppressed. The study of the various components of memory storage—acquisition, consolidation and retrieval—requires a system in which the transgene expression can be turned on and off rapidly. Thus, we have adapted to the brain the rtTA system which uses doxycycline to activate rather than repress transgene expression.

Using the rtTA system in combination with the CaMKIIα promoter, we achieved rapid induction of the expression of both a lacZ reporter gene or a calcineurin transgene in adult mouse brain by administration of doxycycline in the food. The doxycycline-induced transgene expression was restricted to forebrain neurons in hippocampus, cortex and striatum.

As previously reported, we found that overexpression of the calcineurin transgene in the hippocampus, selectively reduced a form of PKA-dependent synaptic plasticity, an intermediate phase of LTP (I-LTP) in CA1 Schaffer collateral pathway (Winder et al., 1998). The present results therefore demonstrate that even transient overexpression of calcineurin is sufficient to produce a deficit in I-LTP, reducing the likelihood that a developmental anomaly contributes to this phenotype. Extending previous findings in the Barnes maze, we also find that spatial memory in the Morris water maze is impaired. The Morris and the Barnes mazes are both hippocampal-dependent tasks that engage similar cognitive processes. On the cued version of both tasks, calcineurin transgene expression did not perturb learning indicating that the transgene did not interfere with visual perception, motivation or motor coordination. Further, the lack of defect on the cued version of the Morris water maze, suggests that the transgene expression probably does not produce a state-dependent effect on performance (Overton, 1964, 1966). However, we cannot exclude the possibility that on the hidden platform version of the task, the induction of the transgene expression may perturb the sensory perception of space rather than the storage or retrieval of spatial memory per se.

The rtTA System Can Be Used to Probe Specific Components of Memory Storage

The flexibility of the rtTA system provides a means to begin to dissect memory into its subcomponents: acquisition, consolidation and retrieval. As a first step towards this direction, we examined the consequence of calcineurin overexpression on memory retrieval, after acquisition and storage were completed. We found that, in addition to its actions on learning and memory, the calcineurin transgene selectively interferes with the retrieval of spatial information. Altogether these results suggest that calcineurin may play a role in both storage and retrieval of spatial memory and that these two processes share some common molecular components.

From a neurobiological perspective, the storage of hippocampal-based explicit memory has been thought to lead to changes in the strength of connections between neurons in the hippocampus and to a consequent alteration in the pattern of neural activity (see for review, Wickelgren, 1979; Squire 1992). According to the constructive view, memory retrieval would require that a retrieval cue creates a distinctive pattern of activity in the hippocampus that recruits and combines with the changes in synaptic strength that occurred during the initial learning process. Each retrieval cue would activate a distinctive synaptic read of memory not simply by passively activating synaptic transmission but by eliciting a newly formed pattern of neuronal activity that may well partake of some transient form of synaptic plasticity (Spear, 1973; Gillund and Shiffrin, 1984; Teyler and DiScenna, 1985; Cai, 1990).

The temporal resolution of the rtTA system allows one to take a genetic approach to the study of retrieval. As a first step in this direction we have asked: Does overexpression of calcineurin interfere with the retrieval of normally acquired memory? We find that the expression of the calcineurin transgene in forebrain selectively impairs the retrieval of spatial information but not the retrieval of non-spatial information. These results suggest that memory retrieval may require some of the molecular components that are recruited for the storage process, although we cannot distinguish whether the failure in retrieval is due to a deficit in retrieval effectiveness or in the retrieval process itself.

Since retrieval can be quite rapid, sometimes seeming almost instantaneous, it is unlikely that the defect in memory retrieval results from a deficit in I-LTP. Indeed, complete abolition of LTP by pharmacological blockade of the NMDA receptor does not block spatial memory retrieval (Morris, 1989). Thus, the molecular component required for retrieval may be critical not for LTP but for a rapid form of neuronal plasticity different from LTP.

Furthermore, since the calcineurin transgene is also expressed modestly in cortex in addition to hippocampus, we cannot assign the defect observed in spatial memory retrieval to either of these brain structures. A richer understanding of the effect of regulated expression of the calcineurin transgene on memory retrieval will require a study of the in vivo activity of hippocampal place cells and an examination of other regulated transgenes with a more restricted expression within the central nervous system.

Our data illustrate that the rtTA system provides a powerful tool for studying the various components of memory. Combined with more specific promoters, this system should allow a detailed dissection of the molecular mechanisms of the various components of memory storage as well as an analysis of the time course of the requirement of hippocampal function, as compared to that of neocortical areas, for the ultimate long-term storage of memory (see Squire, 1992).

Experimental Procedures

Generation of Transgenic Mice

For the generation of rtTA-expressing mice, 8.5 kb of the CaMKIIu promoter (from pMM403, Mayford et al., 1996a) were placed upstream from the rtTA gene (from pUHG-17-1, Gossen et al., 1995) flanked by an artificial intron and splice sites in 5′ and by a polyadenylation signal from SV40 in 3′ (from pNN265, Choi et al., 1991). The CN279 mice were generated as described in Mansuy et al., 1998 using a cDNA encoding a truncated form of the murine calcineurin catalytic subunit Aα, ΔCaM-AI (O'Keefe et al., 1992) placed downstream from the teto-promoter (from pUHD10-3, Gossen and Bujard, 1992). Founder mice were analysed by Southern blotting and PCR and backcrossed to C57BL6 F1/J mice to generate the 1237 and CN279 lines. The generation of tetO-lacZ reporter mice (line lac1) was described in Mayford et al., 1996b. The rTet-lacZ mice were obtained by crossing CaMKIIα promoter-rtTA F1 mice from line 1237 with tetO-lacZ mice from line lac1. To generate the rTet-CN279 mice, CaMKIIa promoter-rtTA F₁ mice from line 1237 were crossed with CN279 F2 or F3 mice (FIG. 14A). The offspring was genotyped by PCR. Mice were administered regular food or food complemented with 6 mg/g of doxycycline (Mutual Pharmaceutical Co.) ad libitum and freshly prepared daily.

β-galactosidase Staining

Brains from adult mice were frozen and cryostat sections (15-20 μm) were prepared. Sections were incubated for 30-60 minutes at 37° C. in X-gal solution containing 1 mg/ml X-gal (Molecular Probes), 5 mM potassium ferricyanide, 5 mM potassium ferrocyanide, 2 mM Mg C₂ in PBS. After staining, sections were washed in PBS for 30 min, fixed in 4% paraformaldehyde for 15-30 min, washed again in PBS, counterstained in 0.5% eosin, rinsed then mounted.

In Situ Hybridization

In situ hybridization were performed as described in Winder et al., 1998. Briefly, cryostat sections were prepared from adult rTet-CN279 brains, fixed 10 min in 4% paraformaldehyde, rinsed in PBS and dehydrated. Sections were rehydrated, permeabilized, washed and rinsed before being hybridized overnight at 37° C. to a [α³⁵S]ATP-labeled oligonucleotide (5′-GCAGGATCCGCTTGGGCTGCAGTTGGACCT-3′) (Seq I.D. No. 5) specific for sequences in pNN265 plasmid. After hybridization, slides were washed, dehydrated then exposed to Kodak Biomax MR® film for 2 to 3 weeks.

Northern Blotting

Northern blot analysis was performed as described in Winder et al., 1998. Briefly, total RNA was prepared from rTet-CN279 forebrains (Chomczynski and Sacchi, 1987). Ten micrograms of RNA were denatured, electrophoresed on 1% agarose gel and transferred to nylon membrane in 0.4 N NaOH. The membrane was hybridized overnight at 42° C. to a radiolabeled 1.1 kb EcoRV-NotI fragment from pNN265, washed and exposed to film for three days.

Phosphatase Assay

Phosphatase assays were performed as described in Winder et al., 1998. Briefly, hippocampal extracts from rTet-CN279 adult mice were prepared in 2 mM EDTA (pH 8), 250 mM sucrose, 0.1% b-mercaptoethanol. Supernatants were incubated at 30° C. for 1 min in the presence of [α³²P]-labeled [Ala97]-RII peptide and either 0.1 mM calmodulin and 0.66 mM Ca²⁽ or 0.33 mM EGTA. The enzyme activity was expressed in nmol Pi released/min/mg protein.

Electrophysiology

Recordings were performed as described in Winder et al., (1998). Briefly, transverse hippocampal slices were equilibrated in oxygenated ACSF (NaCl, 124 mM; KCl, 4.4 mM; CaCl₂, 2.5 mM; MgSO₄, 1.3 mM; NaH₂PO₄, 1 mM; glucose, 10 mM; and NaHCO₃, 26 mM) and subfused (1-2 ml/min) in an interface chamber for 60-90 min at 28° C. Test stimuli were applied at a frequency of 1 per min at an intensity that elicits an EPSP with a slope of 356 of maximum. Slices from mice treated with doxycycline were incubated in ACSF containing doxycycline (6 ng/ml) for 2-3 hours before the tetanus was applied and subfused with doxycycline solution throughout the recordings.

Morris Maze Experiments

Water maze behavioral experiments were performed as described previously (Bourtchouladze et al., 1994). Mice were trained on a visible platform (cued) version of the Morris maze for 2 days with 4 trials per day (60 sec each, different platform and starting position for each trial) where the platform was made visible by a small pipette placed on it. Mice were then either tested 2 weeks later on the cued Morris maze for retrieval or trained on a hidden platform (spatial) version of the task with four trials per day (60 sec each, 30 sec intertrial interval, same platform position but different starting position) for 5 days. Probe trials, where the platform was removed and mice allowed to swim for 60 sec, were performed either immediately after training, 2 weeks later or 4-5 weeks later. Mice were allowed to remain on the platform that was placed back in the training quadrant for 30 sec after each probe trial. The time spent in each quadrant and the number of platform crossings were recorded and plotted for each group of mice. Data on the visible platform version of the task were analyzed with a three-way ANOVA for overall training and retrieval. Data on the hidden platform task were analysed with a two-way ANOVA with one repeated measure and one-way ANOVAs followed by range tests for each of the training day. Data on the probe trials were analysed with a two-way ANOVA followed by one-way ANOVAs and range tests for each of the group across quadrants and for the training quadrant across groups.

References

Abel, T., Nguyen, P. V., Barad, M., Deuel, T. A. S., Kandel, E. R., and Bourtchouladze, R. (1997). Genetic demonstration of a role for PKA in the late phase of LTP and in hippocampus-based long-term memory. Cell 88, 615-626.

Abel, T., Nguyen, P. V., Barad, M., Deuel, T. A. S., Kandel, E. R., and Bourtchouladze, R. (1997). Genetic demonstration of a role for PKA in the late phase of LTP and in hippocampal-based long-term memory. Cell 88, 1-11.

Aggleton, J. P. (1985). One-trial object recognition by rats Quart. J. Exp. Psychol. 37, 279-294.

Alvarez, P., Zola-Morgan, S., and Squire, L. (1994). The animal model of human amnesia: Long-term memory impaired and short-term memory intact. Proc. Natl. Acad. Sci. USA 91, 5637-5641.

Anderson, W. F. (1992) Human Gene Therapy. Science 256:808-813.

Anderson W. F., Blaese M., Culver K. (1990) Human Gene Therapy 1(3):327-62.

Anderson, W. F., Blaese, M., Rosenberg, S. A., Gene Therapy, U.S. Pat. No. 5,399,346, Mar. 21, 1995, filed Mar. 30, 1994, U.S. Ser. No. 220,175.

Bach, M. E., Hawkins, R. D., Osman, M., Kandel, E. R., and Mayford, M. (1995). Impairment of spatial but not contextual memory in CaMKII mutant mice with a selective loss of hippocampal LTP in the range of the theta frequency. Cell 81, 905-915.

Barnes, C. (1979). Memory deficits associated with senescence: A neurophysiological and behavioral study in the rat. J. Comp. Physiol. Psychol. 93, 74-104.

Bartsch., D., Ghirardi, M., Skehel, P. A., Karl, K. A., Herder, S. P., Chen, M., Bailey, C. M. and Kandel, E. R. (1995). Aplysia CREB2 represses long-term facilitation: relief of repression converts transient facilitation into long-term functional and structural change. Cell 83, 979-992.

Bear, M. F., and Abraham, W. C. (1996). Long-term depression in hippocampus. Ann. Rev. Neurosci. 19, 437-462.

Bennett, P. C., Zhao, W., Lawen, A., and Ng, T. G. (1996). Cyclosporin A, an inhibitor of calcineurin, impairs memory formation in day-old chicks. Brain Res. 730, 107-117.

Bito, H., Deisseroth, K., and Tsien, R. W. (1996). CREB phosphorylation and dephosphorylation: A Ca²⁽⁻ and stimulus duration-dependent switch for hippocampal gene expression. Cell 87, 1203-1214.

Bliss, T. V. P., and Collingridge, G. (1993). A synaptic model of memory: long-term potentiation in the hippocampus. Nature 361, 31-39.

Blitzer, R. D., Wong, T., Nouranifar, R., Iyengar, R., and Landau, E. M. (1995). Postsynaptic cAMP pathway gates early LTP in hippocampal CA1 region. Neuron 15, 1403-1414.

Bolshakov, V. Y., Golan, H., Kandel, E. R. and Siegelbaum, S. A. (1997). Recruitment of new sites of synaptic transmission during the cAMP-dependent late phase of LTP at CA3-CA1 synapses in the hippocampus. Neuron 19, 635-651.

Bourtchouladze, R., Frenguelli, B., Blendy, J., Cioffi, D., Schutz, G., and Silva, A. J. (1994). Deficient long-term memory in mice with a targeted mutation of the cAMP-responsive element-binding protein. Cell 79, 59-68.

Brindle, P. K., and Montminy, M. R. (1992). The CREB family of transcription activators. Curr. Opin. Genet. Dev. 2, 199-204.

Cai, Z. -J. (1990). The neural mechanism of declarative memory consolidation and retrieval: A hypothesis. Neurosci. and Behavioral Rev. 14, 295-304.

Cherkin, A. (1969). Kinetics of memory consolidation role of amnesic treatment parameters. Proc. Natl. Acad. Sci. USA 63, 1094-1101.

Choi, T., Huang, M., Gorman C., and Jaenisch, R. (1991). A generic intron increases gene expression in transgenic mice. Mol. Cell. Biol. 11, 3070-3074.

Chomczynski, P., and Sacchi, N. (1987). Single-step method of RNA isolation by acid gnanidinium thiocyanate-phenol-chloroform extraction. Anal. Biochem. 162, 156-159.

Craik, F. I. M., and Lockhart, R. S. (1972). Levels of processing: a framework for memory research. J. Verb. Learn. Verb. Behav. 11, 671-684.

Culver K W, Anderson W F, Blaese R M. (1991) Lymphocyte Gene Therapy, Human Gene Therapy 2(2):107-9.

Culver K W, Osborne W R, Miller A D, Fleisher T A, Berger M, Anderson W F, Blaese R M. (1991) Transplantation Proceedings (1 Pt 1):170-1.

Cummings, J. A., Mulkey, R. M., Nicoll, R. A. and Malenka, R. C. (1996). Ca²⁽ signaling requirements for long-term depression in the hippocampus. Neuron 16, 825-833.

Davis, H. P. and Squire, L. R. (1984). Protein synthesis and memory : a review. Psychol. Bull. 96, 518-559.

Ebbinghaus, H. (1885). Memory: A Contribution to Experimental Psychology. (Dover, New York)

Eichenbaum, H. (1995). The LTP-memory connection. Nature 378, 131-132.

Ennaceur, A., and Delacour, J. (1988). A new one-trial test for neurobiological studies of memory in rats. I: Behavioral data. Behav. Brain Res. 31, 47-59.

Figurov, A., Boddeke, H., and Muller, D. (1993). Enhancement of AMPA-mediated synaptic transmission by the protein phosphatase inhibitor calyculin A in rat hippocampal slices. Eur. J. Neurosci. 5, 1035-1041.

Frieder, B., and Allweis, C. (1982). Memory consolidation: further evidence for the four-phase model from the time courses of diethylthiocarbamate and ethacrinic acid amnesias. Physiol. Behav. 29, 1071-1075.

Gibbs, M. E., and Ng, K. T. (1977). Psychobiology of memory: towards a model of memory formation. Biobehav. Rev. 1, 113-136.

Gillund, G., and Shiffrin, R. M. (1984). A retrieval model for both recognition and recall. Psychol. Rev. 91, 1-67.

Gossen, M., and Bujard, H. (1992). Tight control of gene expression in mammalian cells by tetracycline-responsive promoters. Proc. Natl. Acad. Sci. USA 89, 5547-5551.

Gossen, M., Freundlich, S., Bender, G., Muller, G., Hillen, W., and Bujard, H. (1995). Transcriptional activation by tetracyclines in mammalian cells. Science 268, 1766-1769.

Helekar, S. A. and Patrick, J. (1997). Peptidyl prolyl cis-trans isomerase activity of cyclophilin A in functional homo-oligomeric receptor expression. Proc. Natl. Acad. Sci. USA 94, 5432-5437.

Hogan, B., Beddington, R., Costantini, F., and Lacy, E. (1994). Manipulating the Mouse Embryo, 2nd Edition (Cold Spring Harbour Press: Cold Spring Harbour, New York).

Huang, Y. -Y., and Kandel, E. R. (1994). Recruitment of long-lasting and protein kinase A-dependent long-term potentiation in the CA1 region of hippocampus requires repeated tetanization. Learn. Mem. 1, 74-82.

Huang, Y. -Y., Nguyen, P. V., Abel, T., and Kandel, E. R. (1996). Long-lasting forms of synaptic potentiation in the mammalian hippocampus. Learn. Mem. 3, 74-85.

James, W. (1890). The Principles of Psychology. (New York :Holt).

Kistner, A., Gossen, M., Zimmerman, F., Jerecic, J., Ullmer, C., Labbert, H., and Bujard, H. (1996). Doxycycline-mediated quantitative and tissue-specific control of gene expression in transgenic mice. Proc. Natl. Acad. Sci. USA 93, 10933-10938.

Klee, C. B., Krinks, M. H., Manalan, A. S., Cohen, P., and Stewart, A. A. (1983). Isolation and characterization of bovine brain calcineurin: a calmodulin-stimulated protein phosphatase. Methods Enzymol. 102, 227-244.

Klee, C. B., Draetta, G. F., and Hubbard, M. J. (1987). Calcineurin. Adv. Enzymol. 61, 149-200.

Klee, C. B. (1991). Concerted regulation of protein phosphorylation and dephosphorylation by calmodulin. Neurochem. Res. 16, 1059-1065.

Knight, R. T. (1996). Contribution of human hippocampal region to novelty detection. Nature 383, 256-259.

Kogan, J. H., Frankland, P. W., Blendy, J. A., Coblentz, J., Marowitz, Z., Schutz, G., and Silva, A.J. (1996). Spaced training induces normal long-term memory in CREB mutant mice. Curr. Biol. 7, 1-11.

Kuno, T., Mukai, H., Ito, A., Chang, C. -D., Kishima, K., Saito, N., and Tanaka, C. (1992). Distinct cellular expression of calcineurin Aα and Aβ in rat brain. J. Neurochem. 58, 1643-1651.

Lee, K. A., and Masson, N. (1993). Transcriptional regulation by CREB and its relatives. Biochem. Biophys. Acta 1174, 221-233.

Lisman, J. (1994). The CaM kinase II hypothesis for the storage of synaptic memory. Trends Neurosci. 17, 406-412.

Lu, Y. -F., Tomizawa, K., Morikawa, A., Hayashi, Y., Tokuda, M., Itano, T., Hatase, O., and Matsui, H. (1996). Calcineurin inhibitors, FK506 and cyclosporin A, suppress the NMDA receptor-mediated potentials and LTP, but not depotentiation in the rat hippocampus. Brain Res. 729, 142-146.

Mandel, R. J., Gage, F. H., and Thal, L. J. (1989). Enhanced detection of nucleus basalis magnocellularis lesion-induced spatial learning deficit in rats by modification of training regimen. Behav. Brain Res. 31, 221-229.

Martin, K. C., Michael, D., Rose, J. C., Barad, M., Casadio, A., Zhu, H. and Kandel, E. R. (1997). MAP kinase translocates into the nucleus of the presynaptic cell and is required for long-term facilitation in Aplysia. Neuron 18, 899-912.

Mayford, M., Wang, J., Kandel, E. R., and O'Dell, T. J. (1995). CaMKII regulates the frequency-response function of hippocampal synapses for the production of both LTD and LTP. Cell 81, 891-904.

Mayford, M., Bach, M. E., Huang Y. -Y., Wang, L., Hawkins, R. D., and Kandel, E. R. (1996). Genetic control of Ca²⁺/calmodulin-dependent protein kinase activity in hippocampus and amygdala: Regulated disruption of explicit and implicit memory storage. Science 274, 1678-1683.

Mayford, M., Baranes, D., Podsypanina, K., and Kandel, E. R. (1996a). The 3′ untranslated region of CaMKIIα is a cis-acting signal for the localization and translation of mRNA in dendrites. Proc. Natl. Acad. Sci. USA 93, 13250-13255.

Mayford, M., Bach. M., Huang, Y. -Y., Wang, L., Hawkins, R. D., and Kandel, E. R. (1996b). Control of memory formation through regulated expression of a CaMKII transgene. Science 274, 1678-1683.

Mayford, M., Mansuy, I. M., Muller, R. U., and Kandel, E. R. (1997). Memory and behavior: a second generation of genetically modified mice. Curr. Biol. 7, R580-R589.

McGaugh, J. L. (1968). A multi-trace view of memory storage. Recent advances in learning and memory. (Roma Accademia Nazionale dei Lincei: Rome).

Malenka, R. C., and Nicoll, R. A. (1993). NMDA receptor-dependent synaptic plasticity: multiple forms and mechanisms. Trends Neurosci. 16, 521-527.

Mishkin, M. (1978). Memory in monkeys severely impaired by combined but not by separate removal of amygdala and hippocampus. Nature 273, 297-298.

Morris, R. G. M., Garrud, P., Rawlins, J. N. P., and O'Keefe, J. (1982). Place navigation impaired in rats with hippocampal lesions. Nature 297, 681-683.

Morris, R. G. M. (1989). Synaptic plasticity and learning: selective impairment of learning rats and blockade of long-term potentiation in vivo by the N-methyl-D-aspartate receptor antagnist AP5. J. Neurosci. 9, 3040-3057.

Mulkey, R. M., Endo, S., Shenolikar, S., and Malenka, R. C. (1994). Involvement of a calcineurin/inhibitor-1 phosphatase cascade in hippocampal long-term depression. Nature 369, 486-488.

Muller, D., Hefft, S., and Figurov, A. (1995). Heterosynaptic interactions between LTP and LTD in CA1 hippocampal slices. Neuron 14, 599-605.

Muller, D., Joly, M. and Lynch, G. (1988). Contributions of quisqualate and NMDA receptors to the induction and expression of LTP. Science 242 , 1694-1697.

Mulligan, R. C. (1993). The basic science of gene therapy. Science 260, 926-932.

Mumby, D. G., Pinel, J. P. J., Kornecook, T. J., Shen, M. J., and Redila, V. A. (1995). Memory deficits following lesions of hippocampus or amygdala in rat : Assessment by an object-memory test battery. Psychobiology 23, 26-36.

Myhrer, T., (1988a). Exploratory behavior and reaction to novelty in rats with hippocampal perforant path system disrupted. Behav. Neurosci. 102, 356-362.

Myhrer, T. (1988b). The role of medial and lateral hippocampal perforant path lesions and object distinctiveness in rats reaction to novelty. Physiol. Behav. 42, 3711-377.

O'Keefe, S. J., Tamura, J., Kincaid, R., Tocci, M. J., and O'Neill, E. A. (1992). FK-506 and CsA-sensitive activation of the interleukin-2 promoter by calcineurin. Nature 357, 692-694.

Osten, P., Valsamis, L., Harris, A. and Sacktor, T. C. (1996). Protein synthesis-dependent formation of protein kinase M( in long-term potentiation. J. Neurosci. 16, 2444-2451.

Overman, W. H., Ormsby, G., and Mishkin, M. (1990). Picture recognition versus picture discrimination learning in monkeys with medial temporal removal. Exp. Brain Res. 79, 18-24.

Overton, D. (1964). State-dependent or dissociated learning produced with pentobarbital. J. Comp. Physiol. Psych. 57, 3-12.

Overton, D. (1966). State-dependent learning produced by depressant and atropine-like drugs. Psychopharmacologia. 10, 6-31.

Parsons, J. N., Wiederrecht, G. J., Salowe, S., Burbaum, J. J., Rokosz, L. L., Kincaid, R. L., and O'Keefe, S. J. (1994). Regulation of calcineurin phosphatase activity and interaction with the FK-506-FK-506 binding protein complex. J. Biol. Chem. 269, 19610-19616.

Phillips, R. R., Malamut, B. L., Bachevalier, J., and Mishkin, M. (1988). Dissociation of the effects of inferior temporal and limbic lesions on object discrimination learning with 24-h intertrial intervals. Behav. Brain Res. 27, 99-107.

Paterson, J. M., Smith, S. M., Harmar, A. J., and Antoni, F. A. (1995). Control of a novel adenylyl cyclase by calcineurin. Biochem. Biophys. Res. Comm. 214, 1000-1008.

Raman, I. M., Tong, G. and Jahr, C. E. (1996). β-adrenergic regulation of synaptic NMDA receptors by cAMP-dependent protein kinase. Neuron 16, 415-421.

Reed, J. M. and Squire, L. R. (1997). Impaired recognition memory in patients with lesions limited to the hippocampal formation. Behav. Neurosci. 111, 1-9.

Roberson, E. D., English, J. D., and Sweatt, J. D. (1996). A biochemist's view of long-term potentiation. Learn. and Mem. 3, 1-24.

Rosenzweig, M. R., Bennett, E. L., Colombo, P. J., Lee D. W., and Serrano, P. A. (1993). Short-term, intermediate-term and long-term memories. Behav. Brain Res. 57, 193-198.

Schwaninger, M., Blume, R., Kruger, M., Lux, G., Oetjen, E., and Knepel, W. (1995). Involvement of the Ca²⁽-dependent phosphatase calcineurin in gene transcription that is stimulated by cAMP through cAMP response elements. J. Biol. Chem. 270, 8860-8866.

Scoville, W. B., and Milner, B. (1957). Loss of recent memory after bilateral hippocampal lesions. J. Neurol. Neurosurg. Psychiatry 20, 11-21.

Spear, N. E. (1973). Retrieval of memory in animals. Psychol. Review 80, 163-194.

Squire L. (1987). Memory and Brain (New York: Oxford University Press).

Squire L. (1992). Memory and the hippocampus: A synthesis from findings with rats, monkeys and humans. Psychol. Rev. 99, 195-231.

Stanton, P. K. and Sarvey, J. M. (1984). Blockade of long-term potentiation in rat hippocampal CA1 region by inhibitors of protein synthesis. J. Neurosci. 4, 3080-3088.

Teyler, T. J., and DiScenna, P. (1985). The role of hippocampus in memory: a hypothesis. Neurosci. and Biobehav. Rev. 9, 377-389.

Thomas, M. J., Moody, T. D., Mahinson, M., and O'Dell, T. J. (1996). Activity-dependent β-adrenergic modulation of low frequency stimulation induced LTP in the hippocampal CA1 region. Neuron 17, 475-482.

Tsien, J. Z., Huerta, P. T., and Tonegawa, S. (1996). The essential role of hippocampal CA1 NMDA receptor-dependent synaptic plasticity in spatial memory. Cell 87, 1327-1338.

Tong, G., Shepherd, D., and Jahr, C. E. (1995). Synaptic desensitization of NMDA receptors by calcineurin. Science 267, 1510-1512.

Traynelis, S. F. and Wahl, P. (1997). Control of rat GluR6 glutamate receptor open probability by protein kinase A and calcineurin. J. Physiol. 503, 513-531.

Tully, T., Preat, T., Boynton, S. C., and Del Vecchio, M. (1994). Genetic dissection of consolidated memory in Drosophila. Cell 79, 35-47.

Tulving, E., Markowitsch, H. J., Craik, F. E., Habib, R., and Houle, S. (1996). Novelty and familiarity activations in PET studies of memory encoding and retrieval. Cereb. Cortex 1, 71-79.

Wang, J. -H., and Kelly, P. T. (1996). The balance between postsynaptic Ca²⁽-dependent protein kinase and phosphatase activities controlling synaptic strength. Learn. and Mem. 3, 170-181.

Wang, J. -H., and Kelly, P. T. (1997). Postsynaptic calcineurin activity downregulates synaptic transmission by weakening intracellular Ca2+ signaling mechanisms in hippocampal CA1 neurons. J Neurosci 17, 4600-4611.

Wang, J. -H., and Stelzer, A. (1994). Inhibition of phosphatase 2B prevents expression of hippocampal long-term potentiation. Neuroreport 5, 2377-2380.

Weiskrantz, L. (1970). A long-term view of short-term memory in psychology. Short-term changes in neural activity and behaviour. (Cambridge University Press: England).

Wickelgren, W. A. (1973). The long and the short of memory. Psychol. Bull. 80, 425-432.

Wickelgren, W. A. (1979) Chunking and consolidation: a theoretical synthesis of semantic networks, configuring in conditioning, S-R vs. cognitive learning, normal forgetting, the amnesic syndrome, and the hippocampal arousal system. Psychol. Rev. 86, 44-60

Yakel, J. L. (1997). Calcineurin regulation of synaptic function: from ion channels to transmitter release and gene transcription. Trends Neurosci. 18, 124-134.

Yin, J. C. P., Wallach, J. S., Del Vecchio, M., Wilder, E. L., Zhou, M., Quinn, W. G., and Tully, T. (1994). Induction of a dominant negative CREB transgene specifically blocks long-term memory in Drosophila. Cell 79, 49-58.

Yin, J. C. P., Del Vecchio, Zhou, M., Quinn, and Tully, T. (1995). CREB as a memory modulator:induced expression of a dCREB2 activator isoform enhances long-term memory in Drosophila. Cell. 81, 107-115.

Zhao, W. -Q., Sedman, G. L., Gibbs, M. E., and Ng, .T. (1994). Effect of PKC inhibitors and activators on memory. Behav. Brain Res. 60, 151-160.

Zhao, W. Q., Bennett, P., Sedman, G. L., and Ng, K. T. (1995a). The impairment of long-term memory formation by the phosphatase inhibitor okadaic acid. Brain Res. Bull. 36, 557-561.

Zhao, W. Q., Polya, G. M., Wang, B. H., Gibbs, M. E., Sedman, G. L., and Ng, K. T. (1995b). Inhibitors of cAMP-dependent protein kinase impair long-term memory formation in day-old chicks. Neurobiol. Learning Memory 64, 106-118.

Zola-Morgan, S., and Squire, L. R. (1985). Medial temporal lesions in monkeys impair memory on a variety of tasks sensitive to human amnesia. Behav. Neurosci. 99, 22-34.

Zucker, R. S. (1989). Short-term synaptic plasticity. Annu. Rev. Neurosci. 12, 13-31.

What is claimed is:

1. A transgenic nonhuman mammal whose germ or somatic cells contain a nucleic acid molecule which encodes calcineurin or a variant thereof under the control of a regulatable promoter, introduced into the mammal, or an ancestor thereof, at an embryonic stage.

2. The transgenic nonhuman mammal of claim 1, wherein the regulatable promoter is responsive to a transactivator.

3. The transgenic nonhuman mammal of claim 1, wherein the regulatable promoter is a tetO promoter.

4. The transgenic nonhuman mammal of claim 2, wherein the transactivator is doxycycline.

5. The transgenic nonhuman mammal of claim 2, wherein the transactivator is encoded by a gene under the control of a forebrain specific promoter.

6. The transgenic nonhuman mammal of claim 5, wherein the forebrain-specific promoter is a murine CaMKIIα promoter.

7. The transgenic nonhuman mammal of claim 1, wherein the mammal is a mouse, a rat, a sheep, a bovine, a canine, a porcine or a primate.

8. A screening assay for evaluating whether a compound is effective in improving long-term memory in a subject suffering from impaired long-term memory which comprises:
(a) administering the compound to the transgenic nonhuman mammal of claim 1 or 42 wherein the mammal has increased brain-specific calcineurin activity, and
(b) comparing the long-term memory of the mammal in step (a) with the long-term memory of the mammal in the absence of the compound so as to determine whether the compound is effective in rescuing the long-term memory defect thereby improving the long-term memory of the subject.

9. The screening assay of claim 8, wherein the subject is a human, a rat, a mouse, a sheep, a bovine, a canine, a porcine or a primate.

10. The screening assay of claim 8, wherein the compound is an organic compound, a peptide, an inorganic compound, a lipid or a small synthetic compound.

11. The screening assay of claim 8, wherein the transgenic nonhuman mammal is a genetically modified mouse with increased calcineurin activity in brain.

12. The screening assay of claim 8, wherein the transgenic nonhuman mammal is a lac1 mouse, a 1237 mouse, a CN98 mouse, a CN279 mouse, an rTet-lacZ mouse, or an rTetCN279 mouse.

13. The screening assay of claim 8, wherein the impaired long-term memory of the subject is due to amnesia, Alzheimer's disease, amyotrophic lateral sclerosis, a brain injury, cerebral senility, chronic peripheral neuropathy, a cognitive disability, a degenerative disorder associated with a learning and memory deficit, defective synaptic transmission, Down's Syndrome, dyslexia, electric shock induced amnesia, Guillain-Barre syndrome, head trauma, stroke, cerebral ischemia, Huntington's disease, a learning disability, a memory deficiency, memory loss, a mental illness, mental retardation, memory or cognitive dysfunction, multi-infarct dementia, senile dementia, myasthenia gravis, a neuromuscular disorder, Parkinson's disease, Pick's disease, a reduction in spatial memory retention, senility, Tourrett's syndrome, caridac arrest, open heart surgery, chronic fatigue syndrome, major depression or electroconvulsive therapy.

14. A method for improving long-term memory storage and retrieval in a subject suffering from a long-term memory defect which comprises administering to the subject a compound capable of reversing a defect in intermediate-long-term-potentiation (I-LTP) in the subject thereby improving long-term memory storage and retrieval.

15. A method for improving long-term memory in a subject suffering from a long-term memory defect which comprises administering to the subject a compound identified by the screening assay of claim 8 as effective in improving long-term memory.

16. A method for improving long-term memory in a subject suffering from a long-term memory defect which comprises administering to the subject a compound that inhibits calcineurin activity in the forebrain of the subject thereby improving long-term memory in the subject.

17. A method for improving long-term memory in a subject suffering from a long-term memory defect which comprises administering to the subject an amount of a compound that modifies a calcineurin-dependent biochemical pathway in the forebrain of the subject, effective to modify such pathway and thereby improve long-term memory in the subject.

18. The method of claim 14, 15, 16 or 17 wherein the impaired long-term memory of the subject is due to amnesia, Alzheimer's disease, amyotrophic lateral sclerosis, a brain injury, cerebral senility, chronic peripheral neuropathy, a cognitive disability, a degenerative disorder associated with a learning and memory deficit, defective synaptic transmission, Down's Syndrome, dyslexia, electric shock induced amnesia, Guillain-Barre syndrome, head trauma, stroke, cerebral ischemia, Huntington's disease, a learning disability, a memory deficiency, memory loss, a mental illness, mental retardation, memory or cognitive dysfunction, multi-infarct dementia, senile dementia, myasthenia gravis, a neuromuscular disorder, Parkinson's disease, Pick's disease, a reduction in spatial memory retention, senility, Tourrett's syndrome, caridac arrest, open heart surgery, chronic fatigue syndrome, major depression or electroconvulsive therapy.

19. The method of claim 14, 15, 16 or 17 wherein the compound is an organic compound, a peptide, an inorganic compound, a lipid or a small synthetic compound.

20. The method of claim 14, 15, 16 or 17 wherein the subject is a human, a rat, a mouse, a sheep, a bovine, a canine, a porcine or a primate.

21. The method of claim 14, 15, 16 or 17 wherein the administration is via an aerosol, oral delivery, intravenous delivery, an inhalent, an eyedrop, topical delivery, a time-release implant or an intraspinal injection.

22. The method of claim 21, wherein the implant is subcutaneous.

23. A compound identified by the screening assay of claim 8 as effective in improving long-term memory.

24. A pharmaceutical composition comprising the compound of claim 23 and a carrier.

25. The pharmaceutical composition of claim 24, wherein the carrier is aerosol, topical, intravenous or oral carrier, or a subcutaneous implant.

26. The pharmaceutical composition of claim 25, wherein the implant is a time release implant.

27. A nucleic acid molecule which comprises:
(i) a CaMKIIα promoter sequence or fragment thereof, and
(ii) a nucleic acid sequence encoding a tetracycline-controlled transcriptional activator protein flanked by an artificial intron sequence and splice site sequence in the 5′ direction and by a polyadenylation signal sequence in the 3′ direction.

28. The nucleic acid of claim 27, wherein the nucleic acid sequence of (i) is the sequence of the 8.5 kb CaMKII promoter insert of plasmid pMM403+CAM (from ATCC Accession No.____).

29. The nucleic acid of claim 27, wherein the nucleic acid sequence of (ii) is the sequence of the 1.04 kb insert of plasmid pMM403+rtTA (from ATCC Accession No. ____).

30. The nucleic acid molecule of claim 27, wherein the nucleic acid sequence of (ii) is a rtTA sequence.

31. The nucleic acid molecule of claim 27, wherein (i) is upstream from (ii).

32. A replicable vector which comprises the nucleic acid molecule of claim 27.

33. A host cell which comprises the replicable vector of claim 32.

34. A nucleic acid molecule which comprises:
(i) a transcriptional activator protein-responsive promoter sequence;
(ii) a nucleic acid sequence encoding the Aα catalytic subunit of calcinuerin or a variant thereof;
(iii) a polyadenylation signal sequence.

35. The nucleic acid molecule of claim 34, wherein the nucleic acid sequence of (i) is the sequence of the 1.04 kb insert of plasmid pMM403+rtTA (from ATCC Accession No.____).

36. The nucleic acid molecule of claim 34, wherein the nucleic acid sequence of (i) is the sequence of the 1197 bp insert of plasmid pMM403+CAM (from ATCC Accession No.____).

37. The nucleic acid molecule of claim 34, wherein the sequence of (i) is a tetO promoter sequence.

38. The nucleic acid molecule of claim 34, wherein the sequence of (ii) is truncated a calcineurin ΔCaM-AI.

39. The nucleic acid molecule of claim 34, wherein (i) is upstream of (ii) and (ii) is upstream of (iii).

40. The nucleic acid molecule of claim 34, wherein the nucleic acid sequence of (ii) is operably linked to the promoter of (i).

41. A replicable vector which comprises the nucleic acid molecule of claim 34.

42. A host cell which comprises the replicable vector of claim 41.

43. A transgenic nonhuman mammal whose germ or somatic cells contain the nucleic acid molecule of claim 27 or 34, introduced into the mammal, or an ancestor thereof, at an embryonic stage.

44. A transgenic nonhuman mammal whose germ or somatic cells contain the nucleic acid molecule of claim 27 and 34, introduced into the mammal, or an ancestor thereof, at an embryonic stage.
